(19) United States
(12) Patent Application Publication
(10) Pub. No.: US 2014/0140962 A1
(43) Pub. Date: May 22, 2014

(54) VIRUSES MODIFIED WITH UNNATURAL MOIETIES AND METHODS OF USE THEREOF

(71) Applicants: Isaac Sheridan Carrico, Stony Brook, NY (US); Banerjee Partha, Baltimore, MD (US); Patrick Hearing, St. James, NY (US); Oum Yoon Hyeun, Atlanta, GA (US)

(72) Inventors: Isaac Sheridan Carrico, Stony Brook, NY (US); Banerjee Partha, Baltimore, MD (US); Patrick Hearing, St. James, NY (US); Oum Yoon Hyeun, Atlanta, GA (US)

(73) Assignee: The Research Foundation of State University of New York, Albany, NY (US)

(21) Appl. No.: 14/114,052

(22) PCT Filed: Apr. 26, 2012
(86) PCT No.: PCT/US12/35212

§ 371 Date: Dec. 2, 2013

Related U.S. Application Data
(60) Provisional application No. 61/480,744, filed on Apr. 29, 2011.

(51) Int. Cl. C12N 7/00

(52) U.S. Cl. CPC C12N 7/00
USPC 424/93.6; 435/235.1

(57) ABSTRACT

The invention provides compositions and methods for making and using modified viruses, including infectious viruses, having an external surface linked to at least one heterologous unnatural moiety that is exemplified by unnatural amino acid and unnatural saccharide. The unnatural moiety that is linked to the invention's modified viruses is optionally further linked to a molecule of interest (such as probe, cytotoxin, therapeutic molecule, antibody, affibody, epitope, etc. The invention's compositions and methods for use in, for example, diagnostic applications and therapeutic applications such as gene therapy, oncolytic therapy, and/or vaccine therapy.

This invention was made with government support under grant CBET-1080909 awarded by the National Science Foundation (NSF) and grant R01 AI041636 awarded by the National Institutes for Health (NIH). The government has certain rights in the invention.

### FIELD OF INVENTION

The invention provides compositions and methods for making and using modified viruses, including infectious viruses, having an external surface linked to at least one heterologous unnatural moiety that is exemplified by unnatural amino acid and unnatural saccharide. The unnatural moiety that is linked to the invention's modified viruses is optionally further linked to a molecule of interest (such as probe, cytotoxin, therapeutic molecule, antibody, affibody, epitope, etc.). The invention's compositions and methods find use in, for example, diagnostic applications and therapeutic applications such as gene therapy, oncolytic therapy, and/or vaccine therapy.

### BACKGROUND

Surface modification of virus particles has potential to increase their therapeutic value via tissue selective targeting, modification of immunogenicity, and enabling imaging of bio-distribution. Currently, however, surface tailoring is constrained by a combination of factors including impact on viral fitness, limited access to functionality, or incomplete control over the site of modification.

These difficulties are illustrated by the results with Adenoviruses (Ad) that are widely employed vehicles for gene replacement, gene delivery and vaccine development. As a result of the broad distribution of the coxsackie-adenovirus receptor (CAR), and significant interactions with alternative receptors, most applications benefit from more restrictive adenoviral vector targeting. As a result, significant effort is being invested in both transductional and transcriptional targeting of these vectors. Transductional targeting efforts take advantage of three different methods for capsid remodeling, genetic, non-covalent and chemical modification. Genetic modification of the coat proteins is the most prevalent and has been explored at numerous positions on the adenoviral surface, however due to impact on viral stability and infectivity most studies focus on the HI loop or C-terminus of the fiber protein, the HVR domain within the hexon and the carboxyl terminus of pIX (6, 29). Despite being relatively permissive, genetic alteration of these sites often challenges viral fitness, as evidenced by losses in particle production and infectivity, with such issues determined by the nature and size of modification (8, 15).

Non-covalent adenoviral decoration was first demonstrated with chemically modified anti-CAR antibodies. Advantageously, this strategy allows for both de-targeting, from CAR mediated infection, and retargeting in a single step (3). Such “molecular bridges” between the vector and the cell specific receptor consist of 2 domains, often the first binds specifically to the primary targeting motif in the knob region, effectively masking it, while the second contains receptor specific antibodies, small molecules or peptides (27). However, antibody-ligand conjugation is non-trivial and, more importantly, in vivo stability of the modified adenoviral particle remains a significant question. A conceptually similar technique incorporates the biotin acceptor peptide on the adenoviral fiber as shown by Campos et. al. providing a potentially powerful merging of genetic and adapter based approaches (2, 20).

To broaden the range of accessible functionality, chemical modification of solvent accessible natural residues has been explored. Lysine modification allows a high degree of functionalization with immunosuppressive polymers, targeting polypeptides and imaging reagents. Conjugation of polyethylene glycol (PEG) polymers allows production of “stealth” vectors, which are both protected from immune recognition and effectively de-targeted (9, 12, 19). However, due to the nature of lysine conjugation, control is limited and modification results in a distribution of viral particles with differing surface charges. In addition, accessible functionality is inherently constrained by the nature of amide-bond formation. As a result, modification with proteins, nucleic acids and other nucleophile containing ligands is limited. Alternatively, cysteines have been genetically introduced at exposed locations on the fiber HI loop allowing selective chemical modification (11).

Unnatural amino acids have been introduced into simple capsid protein assemblies derived from bacteriophage, plant viruses or hepatitis B. Such non-covalent homopolymeric structures are produced from E. coli. In addition, a number of papers have described the modification of natural residues on the surfaces of viruses or virus-like particles with azides or alkynes. However, this approach does not provide any of the selectivity advantages of unnatural amino acid incorporation. Also, the simple architecture of these chemically addressable nanoparticles prohibits infection and gene delivery into mammalian cells.

Thus, there remains a need for modification of viral particles that allows specificity in conjugating viral particles to molecules of interest, without adversely impacting viral production and/or infectivity of the modified virus, and preferably without the need for virus genetic modification.

### SUMMARY OF THE INVENTION

The invention provides, in one embodiment, a modified infectious virus having an external surface covalently linked to at least one heterologous unnatural moiety selected from unnatural amino acid and unnatural saccharide. While not intending to limit the type of virus, in one embodiment, the infectious virus is replication competent. In another embodiment, the infectious virus is capable of integrating into the genome of a cell that is susceptible to the virus, and wherein the virus is not replication competent. In a particular embodiment, the external surface comprises one or more of a viral structural protein and a viral structural glycoprotein. In one preferred embodiment, at least one of the unnatural amino acid and the unnatural saccharide comprises one or more chemically reactive group selected from azido group, alkyne group, and a group as shown in FIG. 23. More particularly preferred embodiments include those wherein the chemically reactive group comprises an azido group. For example, in one embodiment, the unnatural amino acid that comprises the azido group comprises azidohomoalanine (AHA). In another embodiment, the unnatural saccharide that comprises the azido group comprises GlcNAz. In a particular embodiment, the unnatural moiety is covalently linked to a molecule of interest.

The invention also provides, in one embodiment, a kit comprising a virus and at least one heterologous unnatural moiety selected from unnatural amino acid and unnatural saccharide.

Further provided by the invention is a method for producing a modified infectious virus having an external surface covalently linked to an unnatural moiety, comprising i) contacting a virus with a host cell susceptible to the virus, wherein the contacting is under conditions for infection of the host cell by the virus to produce an infected cell that comprises the virus, and ii) contacting the infected cell with at least one unnatural moiety selected from unnatural amino acid and unnatural saccharide, wherein the contacting is under conditions for covalently linking an external surface of the virus, that is comprised in the infected cell, with the unnatural moiety. In one embodiment, the susceptible host cell is permissive to the virus, and the modified infectious virus is released from the treated cell. While not required, in some embodiments, the method further comprises determining the level of infection of the treated cells by the modified infectious virus. Also, while not required, in alternative embodiments, the method may further comprise detecting substantially the same level of infectivity of the modified infectious virus as a control virus that lacks the unnatural moiety. In yet another alternative embodiment, the method may further optionally comprise determining the level of replication of the modified infectious virus in the treated cells. Another optional embodiment may include an additional step of detecting substantially the same or greater level of replication of the modified infectious virus compared to a control virus that lacks the unnatural moiety.

The invention also provides a method for detecting disease in a subject comprising contacting tissue of the subject with any one or more of the viruses disclosed herein, wherein a) the unnatural moiety is covalently linked to a molecule of interest, b) the contacting is under conditions for specific binding of the molecule of interest with a second molecule in the tissue, and c) the subject is identified as having disease when an altered level of the specific binding is detected relative to a control normal tissue, and the subject is identified as being disease-free when the level of the specific binding is unaltered relative to a control normal tissue.

The invention additional provides a method for reducing one or more symptoms of disease in a subject, comprising administering to the subject a therapeutic amount of any one or more of the viruses disclosed herein to produce a treated subject, wherein the unnatural moiety is covalently linked to a molecule of interest. Optionally, the method may further comprise determining the level of one or more symptoms of the disease in the treated subject. In another alternative embodiment, the step of administering is under conditions for integration of the virus into the genome of one or more cells in the treated subject. In one embodiment, the disease comprises cancer. While not limiting the type of molecule of interest used in the invention's methods, in one embodiment, the molecule of interest comprises a heterologous antigenic sequence, and wherein the therapeutic amount is an immunologically effective amount.

### BRIEF DESCRIPTION OF THE DRAWINGS

FIG. 1. Schematic representation of adenovirus particle production in the presence of unnatural amino acid (Azidohomoalanine, Aha) resulting in Aha enabled Ad5. Subsequent modification of these azide tags on the viral capsid is possible with the help of Cu catalyzed “click” chemistry (A) with an alkyne probe or Staudinger ligation (B) with a phosphine probe. This probe could theoretically be a small molecule, a peptide, a nucleic acid or even a protein.

FIG. 2. Mass spectrometry characterization of Aha labeled adenovirus particles. Representative spectra for a number of peptides visualized after azide labeled modification. Mass spectra of peptide DGVTPSVALDMTAR obtained from adenoviral hexon protein. The Figure shows ms/ms spectra of the unmodified (A) and modified (B) peptide obtained after LC/MS analysis respectively. Note the mass difference of 5 amu similar to that between methionine and azidohomoalanine.

FIG. 3. Chemical labeling of Aha incorporated into adenovirus particles and effects of unnatural amino acid incorporation on particle production and infectivity. A) Gel scanning analysis of adenovirus particles metabolically labeled with azidohomoalanine and chemically modified with an alk-TAMRA via CuAAC chemistry (for lanes 1-3 chemical labeling is performed on intact virus particles; lane 4 shows “click” labeling on denatured virus). B) Particle count of virus produced in the presence of azidohomoalanine, methionine, a mixture of Aha and Met and without metabolic label. Labeling carried out by pulsing with Aha or Met supplemented media between 18 to 24 hours post-infection using 4 mM concentrations of surrogate amino acid. Particle counts are measured by absorbance at 260 nm. C) Infectivity of azide enabled viral particle by plaque forming assay. 293 cells infected with 10³ azide-Ad5 particles. Infected cells were overlaid with bactoagar containing Neutral Red and plaques counted 7 days after initial infection.

FIG. 4. Increasing concentrations of Aha shows higher levels of azide incorporation without substantial loss of viral fitness. A) Western blot of virus particle produced with increasing Aha concentration from 8 mM to 56 mM. The particles were modified with a phosphine-FLAG reacted for 2 hours and probed with an anti-FLAG antibody. B) Fluorescent gel scan of virus particles labeled with alkyne-TAMRA (a rhodium dye) produced at 2 different concentrations of Aha, 4 mM and 32 mM. Standard concentrations of TAMRA were used to determine the number of dye molecules coupled to each viral particle. Based on the protein marker the Hexon, Penton and Fiber appear to being labeled with Aha. Total number of dyes per particle was calculated by estimating number of label for each of these three proteins. C) Infectivity of increasing Aha incorporated adenoviral particles was assayed by plaque forming ability as compared to methionine (Met) labeled controls.

FIG. 5. Viral particles metabolically labeled and chemically modified used to target a murine breast cancer cell line, 4T1. A) Structure of alkyne-PEG-Folate used to modify azide enabled adenovirus capsid encoding a GFP transgene. B) Fluorescence microscopy of 4T1 cells infected with 4 mM and 32 mM Aha labeled virus and metabolically unlabeled virus that have subsequently been modified with alkyne-peg-folate using “click” chemistry. Cells were infected at an MOI of 50, and images captured 24 hours post infection. Microscopy was carried out on a glass bottom dish mounted on a Zeiss LSM 510 microscope. Bright field images were taken with a GFP filter.

FIG. 6. Quantification of virus particles targeted towards a murine breast cancer cell line, 4T1 with and without competing free ligand. A) Luciferase expression of 4T1 cells 24 hours post infection with azide labeled (4 mM and 32 mM), methionine labeled and metabolically unlabeled virus particles (Ad-Luciferase) all treated with alkyne-peg-folate. Luciferase activity was measured using a Perkin Elmer chemiluminescence plate reader (ex: 485±10 nm; em: 528±10 nm). B) Luciferase activity measurement as in part A using virus particles labeled with 32 mM Aha but this time infection carried out in presence of external folic acid 0 mg/L, 0.1 mg/L and 1 mg/L.

FIG. 7. Synthesis of Azidohomoalanine.

FIG. 8. Synthesis of Alk-PEG-Folate.

FIG. 9. Determination of adenoviral protein production during labeling with Aha. Infected cells grown in the presence of Aha, Met, Aha+Met or unlabeled from 18 to 24 hours post infection were lysed 24 hours post infection and protein production probed using an anti-penton antibody. An IR680 dye was used as secondary. The blots were visualized using an Odyssey LICOR, excitation at 680 and emission at 700±15 nm.

FIG. 10. Determination of adenoviral protein production during labeling with increasing Aha concentration as compared to increasing Met concentration. Infected cells grown in the presence of different concentrations of Aha or Met, 18 to 24 hours post infection were lysed 24 hours post infection and protein production probed using an anti-penton antibody. An IR680 dye was used as secondary. The blots were visualized using an Odyssey LICOR, excitation at 680 and emission at 700±15 nm.

FIG. 11. Determination of labeling of Aha enabled virus with alkyne-fluorophore by Cu catalyzed “click” chemistry. A) Fluorescent gel scanned image of Aha enabled virus reacted with alkyne-TAMRA (well 6 and 7) and unlabeled virus reacted with alkyne-TAMRA (well 8). The “click” reaction was run overnight in a deoxygenated glove bag. Well 1-5: Decreasing concentrations of standard dye (TAMRA) for determining the concentration of label. The gel was run at 200V for 1 hour at 4° C. and scanned within 10 minutes of the end of run. B) Standard curve drawn between fluorescence intensity and concentration of dye loaded on gel. Slope of graph was used to determine concentration of labeled protein.

FIG. 12. Azide incorporation pattern of Aha enabled virus with different labeling times. Purified viral particles have subsequently been modified with alkyne-TAMRA by Cu catalyzed “click” chemistry. Virus produced by Aha labeling between 10 to 16 hours post infection (lane 1) show shifts in Aha incorporation and staggered labeling percentage when compared to Ad5 particles produced by labeling between 18 to 24 hours post infection (lanes 1, 3 and 4).

FIG. 13. A schematic illustrating incorporated O-GlcNAz residue on the adenoviral fiber protein and its subsequent chemical modification with ligands. Potential sites of O-GlcNAz incorporation are indicated in red circles. Either “click” (A) or Staudinger ligation (B) chemistries were used to decorate metabolically labelled adenovirus.

FIG. 14. Qualitative analysis of azido sugar incorporation into the fiber protein of hAd5. A) Anti-FLAG Western of non-denatured viral particles treated with alkyne-FLAG under CuAAC conditions (100 mM Tris pH=8; 1 mM CuBr; 3 mM Bathophenanthroline disulphonic acid disodium salt; 400 μM alk-FLAG; 12 hr; RT). Top: anti-FLAG Western. Bottom: Coomassie. B) Fluorescent analysis of denatured O-GlcNAz-enabled viral particles treated with alkyne-TAMRA under CuAAC conditions (ex: 532; em: 580 BP 30) C) Hexosaminidase treatment (5 μg/μL Hex-C; 37° C. overnight) and subsequent Staudinger modification of partially purified O-GlcNAz labeled fiber protein (100 mM Tris pH=8; 400 μM phos-FLAG; 2 hr; RT). Top: anti-FLAG Western. Bottom: Coomassie.

FIG. 15. Effective gene transduction of 4T1 cells with retargeted Ad5. A) Structure of alkyne-folate ligand. See FIG. 21. B) GFP fluorescence microscopy images of alkyne-folate modified metabolically labeled Ad5 virus and metabolically unlabeled Ad5 infected 4T1 cells. Microscopy was carried out on Glass Bottom dishes using a Zeiss LSM 510 fluorescence microscope. Cells were infected at an MOI of 50 pfu/cell and images taken 24 hours post infection. (lanes i: 4T1 cells infected with folate modified Ac₄GalNAz labeled Ad5; lane ii: 4T1 cells infected with metabolically unlabeled Ad5 (no Ac₄GalNAz); lane iii: 4T1 cells infected with folate unmodified Ac₄GalNAz labeled Ad5 (no alkyne-folate); lane iv: 4T1 cells infected with folate modified Ac₄GalNAz labeled Ad5 pre-treated with 1 mg/L folic acid).

FIG. 16. Azide enabled adenoviral fitness, viral particle production and infectivity. A) Number of adenoviral particles obtained by labeling growth media with Ac₄GalNAz, Ac₄GlcNAz or no exogenous sugar. Viral particle count is obtained by measuring absorbance at 260 nm with 1OD=1×10¹² particles/mL. B) Plaque assay comparing infectivity of Ad5 labeled by Ac₄GalNAz, Ac₄GlcNAz or no exogenous sugar in the growth media. Infectivity was assessed by counting no of plaques produced upon infection of 293 cells with labeled virus.

FIG. 17. Determination of labeling of azido sugar enabled virus with alkyne-fluorophore by Cu catalyzed “click” chemistry. A) Fluorescent gel scanned image of azido sugar enabled virus reacted with alkyne-TAMRA (well 6) and unlabeled virus reacted with alkyne-TAMRA (well 7). The “click” reaction was run overnight in a deoxygenated glove bag. Well 1-5: Decreasing concentrations of standard dye (TAMRA) for determining the concentration of label. The gel was run at 200V for 1 hour at 4° C. and scanned within 10 minutes of the end of run. B) Standard curve drawn between fluorescence intensity and concentration of dye loaded on gel. Slope of graph was used to determine concentration of labeled protein.

FIG. 18. Effective gene transduction of 4T1 cells with retargeted Ad5. Gene delivery efficiencies (GFP) of alkyne-folate modified Ad5 in the presence or absence of exogenous free folate measured using a Synergy 2 fluorescence plate reader (murine breast cancer cell line (4TI); MOI=50; 24 hr post-infection; ex: 485±10 nm; em: 528±10 nm). The results are shown as the mean±standard deviation of the percent relative transduction of at least three independent observations. *, P<0.05.

FIG. 19: Synthesis of 1,3,4,6-tetra-O-acetyl-N-azidiacetyl-α,β-D-glucosamine.

FIG. 20: Synthesis of 1,3,4,6-tetra-O-acetyl-N-azidoacetyl-α,β-D-galactosamine.

FIG. 21: Synthesis of Alkyne-folate.

FIG. 22: Exemplary unnatural saccharide moieties.

FIG. 23: Exemplary chemically reactive groups that may be conjugated to the invention's unnatural amino acids and/or unnatural saccharides.

FIG. 24. A cartoon illustrating adenovirus particles chemically modified with both a folate moiety, via Staudinger conjugation with an introduced O-GlcNAz, and SB-T-1214, via “click” modification of metabolically introduced homopropargylglycine.

FIG. 25. Chemoselective modification of GalNAz and HPG (an amino acid containing an alkyne reactive group) labeled adenovirus with Phosphine-FLAG and az-TAMRA. A) Anti-FLAG western blot of peptide and TAMRA labeled Ad, demonstrating incorporation of azido sugar onto adenoviral fiber. B) Fluorescent analysis of TAMRA labeled Ad indicates HPG incorporation onto virus capsid.

FIG. 26. Viral fitness analysis. Adenoviral particle count was assessed after purification (purple bars) as assayed by UV detection showing efficient particle generation in presence of different non-natural substrates. Virus plaque assay (blue bars) showing infectivity of modified virus particles.

FIG. 27. MTT assay to determine cytotoxic synergism of SBT-AdTRAIL. Comparison of cytoxicity profiles of metabolically “unarmed” AdTRAIL, “armed” SBT-AdTRAIL and free SB-T-1214 on ovarian carcinoma cells (ID8). 5 days post-treatment MTT assay was performed to determine cell death.

FIG. 28. Targeting analysis of folate/SBT-AdLuc. Luciferase analysis of 4T1 cells 24 hours post infection with modified virion both in the presence and absence of external folic acid showing dose dependent gene expression.

FIG. 29 Structures of az-SBT1214 and phosphine-folate used to chemoselectively modify O-GlcNAz and HPG labeled Ad-Luc.

### DEFINITIONS

To facilitate understanding of the invention, a number of terms are defined below. Further definitions appear throughout the text.

The term “recombinant” nucleotide sequence refers to a nucleotide sequence that is produced by means of molecular biological techniques (e.g., cloning, enzyme restriction and/or ligation steps) and/or chemical synthesis.

“Recombinant protein” or “recombinant polypeptide” refers to a protein molecule that is expressed using a recombinant nucleotide sequence.

“Recombinant mutation” refers to a mutation that is introduced by means of molecular biological techniques. This is in contrast to mutations that occur in nature.

“Recombinant virus” refers to a virus that contains a recombinant nucleotide sequence, recombinant polypeptide, and/or recombinant mutation, as well as progeny of that virus.

“Endogenous,” “wild type,” “wildtype,” “wt” and “wild-type” when in reference to a molecule (e.g., moiety and/or sequence that is introduced to a cell and/or virus) refers to a molecule as it occurs in nature (e.g., in the cell and/or virus). It is now appreciated that most or all gene loci exist in a variety of allelic forms, which vary in frequency throughout the geographic range of a species. Thus, in one embodiment, a “wild type” sequence is the sequence that occurs at the highest frequency in nature.

The term “heterologous” when in reference to a molecule (e.g., moiety and/or sequence that is introduced to a cell and/or virus) refers to a molecule that is not endogenous (to the cell and/or virus to which it is introduced). For example, a “heterologous” gene refers to a gene that is not in its natural environment (in other words, has been altered by the hand of man). For example, a heterologous gene includes a gene from one species introduced into another species. A heterologous gene also includes a gene native to an organism that has been altered in some way (for example, mutated, added in multiple copies, linked to a non-native promoter or enhancer sequence, etc.). Heterologous genes may comprise cDNA forms of a gene; the cDNA sequences may be expressed in either a sense (to produce mRNA) or anti-sense orientation (to produce an anti-sense RNA transcript that is complementary to the mRNA transcript). Heterologous genes are distinguished from endogenous genes in that the heterologous gene sequences are typically joined to nucleotide sequences comprising regulatory elements such as promoters that are not found naturally associated with the gene for the protein encoded by the heterologous gene or with gene sequences in the chromosome, or are associated with portions of the chromosome not found in nature (for example, genes expressed in loci where the gene is not normally expressed).

“Portion” and “fragment” when made in reference to a nucleic acid sequence or protein sequence refer to a piece of that sequence that may range in size from two (2) contiguous nucleotides and amino acids, respectively, to the entire sequence minus one nucleotide and amino acid, respectively. Thus, “at least a portion of” a nucleic acid sequence or protein sequence refers to a piece of that sequence that may range in size from two (2) contiguous nucleotides and amino acids, respectively, to the entire sequence.

“Chimeric,” “fusion” and “hybrid” composition (e.g., when in reference to an amino acid sequence, nucleotide sequence, virus, cell, etc.) refers to a composition containing parts from different origins. In one embodiment, the parts may be from different organisms, different tissues, different cells, different viruses, etc. In another embodiment, the parts may be from different proteins and/or genomic sequences from the same organism, same tissue, same cell, same virus, etc.

The terms “lack” and “lacking” a nucleotide sequence when made in reference to a vector means that the vector contains at least one deletion (i.e., absence of one or more nucleotides) in the nucleotide sequence. Deletions may be continuous (i.e., uninterrupted) or discontinuous (i.e., interrupted). Deletions may lie in a coding sequence or a regulatory sequence. A deletion can be a partial deletion (i.e., involving removal of a portion ranging in size from one (1) nucleotide residue to the entire nucleic acid sequence minus one nucleic acid residue) or a total deletion of the nucleotide sequence. Deletions are preferred which prevent the production of at least one expression product encoded by the nucleotide sequence. For example, a vector that lacks adenovirus E1 gene region refers to a vector that contains at least one deletion in the E1 gene region. Preferably, though not necessarily, the deletion prevents the production of at least one of the multiple proteins encoded by the E1 gene region.

“Virus” refers to an obligate, ultramicroscopic, intracellular parasite particle of nucleic acid sequence (DNA or RNA) that is assembled inside a polypeptide and/or glycoprotein “shell” (also referred to as “envelope” or “coat”), and that is incapable of autonomous replication (i.e., replication requires the use of a host cell's machinery). Viruses are exemplified by adenovirus, retrovirus, herpes simplex virus, poxvirus, adeno-associated virus, baculovirus, measles virus, lentivirus, oncovirus (e.g., retroviruses, lentiviruses, poxviruses and herpes viruses), hybrid virus, and recombinant virus.

The term “replication” of a virus includes, but is not limited to, the steps of adsorbing (e.g., receptor binding) to a cell, entry into a cell (such as by endocytosis), introducing its genome sequence into the cell, un-coating the viral genome, initiating transcription of viral genomic sequences, directing expression of viral encapsidation proteins, encapsidating of the replicated viral nucleic acid sequence with the encapsidation proteins into a viral particle that is released from the cell to infect other cells that are of a permissive and/or susceptible character. A virus may be infectious (i.e., can penetrate a cell) without being replication competent (i.e., fails to release virions from the infected cell).

“Replication competent” and “RC” when in reference to a viral vector and/or virus means capable of adsorbing (e.g., receptor binding) to a cell, entry into a cell (such as by endocytosis), introducing its genome sequence into the cell, un-coating the viral genome, initiating transcription of viral genomic sequences, directing expression of viral encapsidation proteins, encapsidating of the replicated viral nucleic acid sequence with the encapsidation proteins into new progeny virus particles.

“Replication incompetent,” “replication defective,” “replication attenuated” are used interchangeably to refer to a virus and/or viral vector that has a reduced level of replication compared to wild type virus and/or to a viral vector containing wild type virus nucleotide sequences. Replication incompetent also means a virus particle that is substantially incapable of completing one or more of the steps of replication. Methods for producing replication incompetent adenoviral vectors are known in the art (e.g., U.S. Pat. No. 7,300,657 to Pau, U.S. Pat. No. 7,468,181 to Vogels, U.S. Pat. No. 6,136,594 to Dalemans, U.S. Pat. No. 5,994,132 to Chamberlain et al., U.S. Pat. No. 6,797,265 to Amalfitano et al., U.S. Pat. No. 7,563,617 to Hearing et al., and U.S. Pat. No. 6,262,035 to Campbell et al.). For example, in one embodiment, a replication incompetent adenovirus and/or adenoviral vector (a) lacks (i.e., has a deletion of) adenovirus E1 gene coding sequence, (b) lacks adenovirus E1 gene coding sequence and E2b gene coding sequence (c) lacks adenovirus E1 gene coding sequence and adenovirus E4 gene coding sequence, (d) lacks adenovirus E1 gene coding sequence and adenovirus Eta gene coding sequence, and/or (e) lacks adenovirus E1 gene coding sequence and adenovirus EIVa2 gene coding sequence.

“Infection” and “infectious” when in reference to a virus refer to adsorption of the virus to the cell and penetration into the cell. A virus may be infectious (i.e., can adsorb to and penetrate a cell) without being replication competent (i.e., fails to produce new progeny virus particles).

A “non-infectious” and “uninfectious” virus is a virus that is incapable of adsorption to, and/or penetration into, a cell.

“Productive” virus is a replication competent virus that is capable of a “productive infection,” i.e., wherein the replication competent virus produces new progeny virus particles that are released extracellularly. Productive infection by a productive virus may be detected by detection of CPE.

“Non-productive” virus is a replication competent virus that produces a “non-productive infection,” i.e., wherein the replication competent virus produces new progeny virus particles that are not released from the infected cell. This includes scenarios where the viral genome is integrated into the host cell genome. Non-productive infection by a non-productive virus may be detected by detecting virus proteins and/or nucleic acids in cellular extracts, in the absence of CPE.

“Encapsidated” when made in reference to a nucleotide sequence refers to a nucleotide sequence that is packaged inside a protein envelope to form a particle. Data presented herein demonstrates that the invention's nucleotide sequence vectors were packaged efficiently into stable virus particles. Encapsidated vectors of the invention may be recovered following transfection or infection of target cells using methods known in the art. When used herein, “recovering” encapsidated vectors refers to the collection of the vectors by, for example, lysis of the cell (e.g., freeze-thawing) and removing the cell debris by pelleting, and/or collection of extracellular solutions.

The terms “cytopathic effect” and “CPE” as used herein describe changes in cellular structure (i.e., a pathologic effect). Common cytopathic effects include cell destruction, syncytia (i.e., fused giant cells) formation, cell rounding, vacuole formation, and formation of inclusion bodies. CPE results from actions of a virus on permissive cells that negatively affect the ability of the permissive cellular host to perform its required functions to remain viable. In in vitro cell culture systems, CPE is evident when cells, as part of a confluent monolayer, show regions of non-confluence after contact with a specimen that contains a virus. The observed microscopic effect is generally focal in nature and the foci are initiated by a single virion. However, depending upon viral load in the sample, CPE may be observed throughout the monolayer after a sufficient period of incubation. Cells demonstrating viral induced CPE usually change morphology to a rounded shape, and over a prolonged period of time can die and be released from their anchorage points in the monolayer. When many cells reach the point of focal destruction, the area is called a viral plaque, which appears as a hole in the monolayer. The terms “plaque” and “focus of viral infection” refer to a defined area of CPE which is usually the result of infection of the cell monolayer with a single infectious virus which then replicates and spreads to adjacent cells of the monolayer. Cytopathic effects are readily discernable and distinguishable by those skilled in the art.

“Integration” of a first nucleotide sequence (e.g., a transgene) into a second nucleotide sequence (e.g., a genome) refers to the insertion of the first nucleotide sequence at one or more locations (referred to as “integration sites”) within the second nucleotide sequence following contacting the first and second nucleotide sequences. “Efficiency of integration” refers to the number of inserted first nucleotide sequences relative to the number of first nucleotide sequences that were contacted with the second nucleotide sequence. Methods for determining efficiency of integration are known in the art (McCarty et al. (2004) Annual Review of Genetics 38:819-844), including quantitative real-time PCR assays (Huser et al. (2002) J. Virol. 76:7554).

“Site-specific integration” and “SSI” refer to the insertion of the first nucleotide sequence occurs at one or more particular locations (“integration sites”) in the second nucleotide sequence. In one embodiment, site-specific integration of a transgene into chromosome 19 AAVS1 sites may be effected by using Rep68/78 proteins in trans to the transgene and an “Rep Binding Element” (“RBE”) in cis. A 16 bp Rep Binding Element is Sufficient for Mediating Rep-dependent Integration into AAVS1. This RBE can be found either within the AAV Terminal Repeat or the p5 Integration Efficiency Element (p5IEE).

“Gene therapy” refers to reducing one or more clinical and/or sub-clinical symptoms of disease in a subject by insertion of nucleotide sequences into the subject's cells to replace damaged or abnormal genes with normal ones, and/or to provide new genetic instructions to help fight disease. Viruses are used as gene delivery vectors, as exemplified by vectors using sequences from adenovirus, adeno-associated virus, herpes simplex virus, retrovirus, lentivirus, baculovirus, etc., as described herein.

The term “control” as used herein when in reference to a sample (e.g., cell, tissue, animal, virus, etc.) refers to any type of sample that one of ordinary skill in the art may use for comparing to a test sample (e.g., cell, tissue, animal, virus, etc.) by maintaining the same conditions in the control and test samples, except in one or more particular factors. In one embodiment, the comparison of the control and test samples is used to infer a causal significance of this varied one or more factors.

A “subject” that may benefit from the invention's methods includes any multicellular animal, preferably a mammal. Mammalian subjects include humans, non-human primates, murines, ovines, bovines, ruminants, lagomorphs, porcines, caprines, equines, canines, felines, ayes, etc.). Thus, mammalian subjects are exemplified by mouse, rat, guinea pig, hamster, ferret and chinchilla. The invention's compositions and methods are also useful for a subject “in need of reducing one or more symptoms of” a disease includes a subject that exhibits and/or is at risk of exhibiting one or more symptoms of the disease. For Example, subjects may be at risk based on family history, genetic factors, environmental factors, etc. This term includes animal models of the disease. Thus, administering a composition (which reduces a disease and/or which reduces one or more symptoms of a disease) to a subject in need of reducing the disease and/or of reducing one or more symptoms of the disease includes prophylactic administration of the composition (i.e., before the disease and/or one or more symptoms of the disease are detectable) and/or therapeutic administration of the composition (i.e., after the disease and/or one or more symptoms of the disease are detectable). The invention's compositions and methods are also useful for a subject “at risk” for disease refers to a subject that is predisposed to contracting and/or expressing one or more symptoms of the disease. This predisposition may be genetic (e.g., a particular genetic tendency to expressing one or more symptoms of the disease, such as heritable disorders, etc.), or due to other factors (e.g., environmental conditions, exposures to detrimental compounds, including carcinogens, present in the environment, etc.). The term subject “at risk” includes subjects “suffering from disease,” i.e., a subject that is experiencing one or more symptoms of the disease. It is not intended that the present invention be limited to any particular signs or symptoms. Thus, it is intended that the present invention encompass subjects that are experiencing any range of disease, from sub-clinical symptoms to full-blown disease, wherein the subject exhibits at least one of the indicia (e.g., signs and symptoms) associated with the disease.

“Subject in need of reducing one or more symptoms of” a disease, e.g., infection with a pathogen, includes a subject that exhibits and/or is at risk of exhibiting one or more symptoms of the disease. For Example, subjects may be at risk based on family history, genetic factors, environmental factors, etc. This term includes animal models of the disease.

A “susceptible” cell refers to a cell that is capable of being infected by a virus. “Infection” refers to adsorption of the virus to the cell and penetration into the cell. Susceptible cells include permissive and non-permissive cells. Susceptibility of a cell to a virus may be determined by detecting the presence of virus proteins and/or virus RNA and/or virus DNA in extracts of infected cells.

A “permissive” cell refers to a cell that is both susceptible to a virus and capable of supporting replication of viral nucleic acid sequences and/or viral peptide sequences. While not required, in one embodiment, a cell is permissive if the replicated viral nucleic acid sequences and viral peptide sequences are assembled into a virion particle. In some embodiments, a permissive cell releases the assembled virions contained therein. In other embodiments, the assembled virions remain inside the permissive cells without release. Viral replication may be determined by, for example, production of viral nucleic acid sequences and/or of viral peptide sequences. Production of progeny virus may be determined by observation of a cytopathic effect. However, this method is less preferred than detection of virus nucleotide sequences, since a cytopathic effect may not be observed even when viral replication is detectable by detecting virus nucleotide sequences.

A cell that is “not permissive” (i.e., “non-infections”) is a cell that is not capable of supporting viral replication.

“Modify,” “modified” when in reference to a virus, refers to a virus any part of which (such as the nucleic acid sequence and/or amino acid sequence and/or saccharide (e.g., saccharide of a glycoprotein)) has been altered by human manipulation using any method (e.g., chemical, biochemical, and/or molecular biological techniques) compared to the virus (e.g., wild-type virus) prior to human

The term “surface” refers to one or more of the faces of a three-dimensional object. For example, a virus “external surface” refers to the exterior face of the virus polypeptide and/or glycoprotein “shell” (also referred to as “envelope” or “coat”), which is distal to the virus genetic nucleic acid sequence. In contrast, a virus “interior surface” refers to the inner face of the virus polypeptide and/or glycoprotein shell, which is proximal to the virus genetic nucleic acid sequence.

“Saccharide” refers to a carbohydrate, including a simple sugar (a monosaccharide) and poly saccharides that contain two or more simple sugars. Glucose, lactose, and cellulose are saccharides.

“Unnatural,” “non-natural” and “analog” when in reference to a molecule are interchangeably used to refer to a molecule other than that which occurs in nature, such as an “unnatural amino acid” and “unnatural saccharide.”

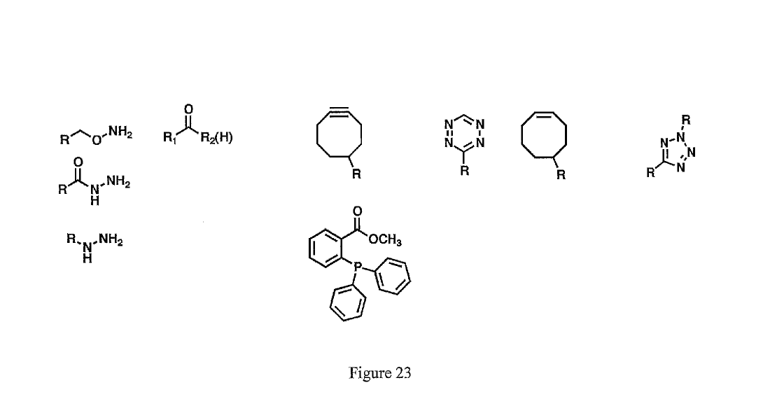

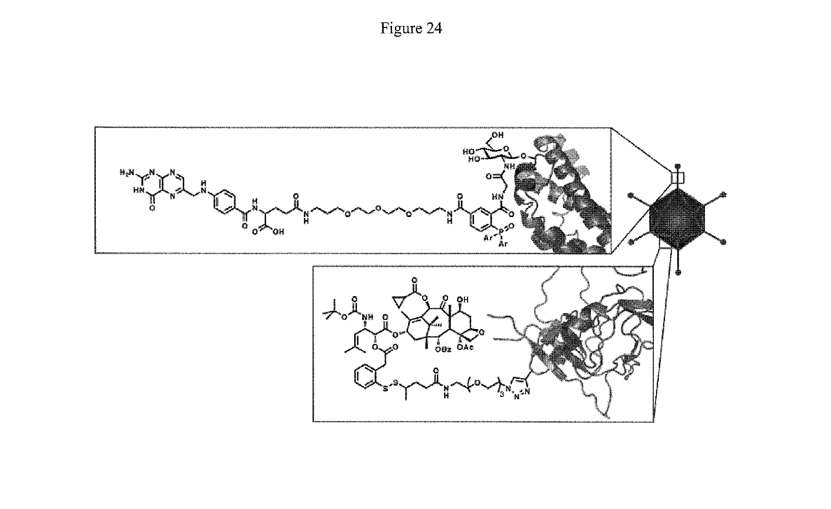

For example, an “unnatural amino acid” refers to an amino acid other than the twenty amino acids that are universal in nature and encoded by wild-type codons. Unnatural amino acid is exemplified by an amino acid containing one or more chemically reactive group, exemplified by azido group, alkyne group, and a group as shown in FIG. 23. Unnatural amino acid is also exemplified by azidonorleucine, 3-(1-naphthyl)alanine, 3-(2-naphthyl)alanine, p-ethynyl-phenylalanine, p-propargly-oxy-phenylalanine, m-ethynyl-phenylalanine, 6-ethynyl-tryptophan, 5-ethynyl-troptophan, (R)-2-amino-3-(4-ethynyl-1H-pyrol-3-yl)propanic acid, p-bromophenylalanine, p-idiophenylalanine, p-azidophenylalanine, 3-(6-chloroindolyl)alanine, 3-(6-bromoindolyl)alanine, 3-(5-bromoindolyl)alanine, azidohomoalanine, and p-chlorophenylalanine. Unnatural amino acids are also exemplified by homoallyglycine, homoproparglycine, norvaline, norleucine, cis-crotylglycine, trans-crotylglycine, 2-aminoheptanoic acid, 2-butynylglycine, allylglycine, azidoalanine, azidohomoalanine (described in Tirrell et al., U.S. Pat. No. 7,198,915), and L-homopropargylglycine (HPG). Additional exemplary unnatural amino acids are described in Link et al., Current Opinion in Biotechnology 14, 603 (December, 2003) and Liu et al., in Annual Review of Biochemistry, 79: 413-444]. In a particularly preferred embodiment, the unnatural amino acid is an azido amino acid exemplified by azidohomoalanine (“AHA”). Methods for incorporating unnatural amino acids into proteins are described in Link et al. (December, 2003) and Liu et al., in Annual Review of Biochemistry, 79:413-444]. Methods for conjugating the unnatural amino acids with molecules of interest (e.g., fluorophores, anti-cancer agents, etc.) are known in the art (Sawa et al., PNAS (Aug. 15, 2006), Beatty et al., Angewandte Chemie-International Edition 45, 7364 (2006), Dieterich et al., Nature Neuroscience 13, 897 (July, 2010), Dieterich et al., Nature Protocols 2, 532 (2007), Dieterich et al., J. Graumann, D. A. Tirrell, E. M. Schuman, Proceedings of the National Academy of Sciences of the United States of America 103, 9482 (Jun. 20, 2006), Dieterich et al., J. Graumann, E. M. Schuman, Molecular & Cellular Proteomics 6, 20 (August, 2007), and Ngo et al., Nature Chemical Biology 5, 715 (October, 2009)). Additional methods for modifying the invention's unnatural amino acids are known in the art (Datta et al., Journal of the American Chemical Society 124, 5652 (May 22, 2002), Mahal et al., Science 276, 1125 (May 16, 1997), and Yarema et al., Glycobiology 6, 801 (October, 1996)).

An “unnatural saccharide” refers to a saccharide other than a saccharide that occurs in nature, and is exemplified by a saccharide containing one or more chemically reactive group, exemplified by azido group, alkyne group, and a group as shown in FIG. 23. For example, unnatural saccharide includes a saccharide having the formula

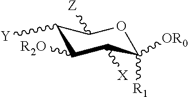

### R₀═H or COCH₃

### R₁═H or CO₂H

X═NHCO(CH₂)ₙN₃ or NHCO(CH₂)ₙ-alkyne or H or OH or OCOCH₃

### R₂═H or COCH₃ or CH(CO₂H)CH₃

Y═OH or OCOCH₃ or NHCO(CH₂)ₙN₃ or NHCO(CH₂)ₙ-alkyne
Z═CH₂OH or CH₂OCOCH₃ or CH(OH)CH(OH)CH₂OH or (CH₂)ₙN₃ or (CH₂)ₙ-alkyne
n=natural number (1, 2, 3, 4 . . . )

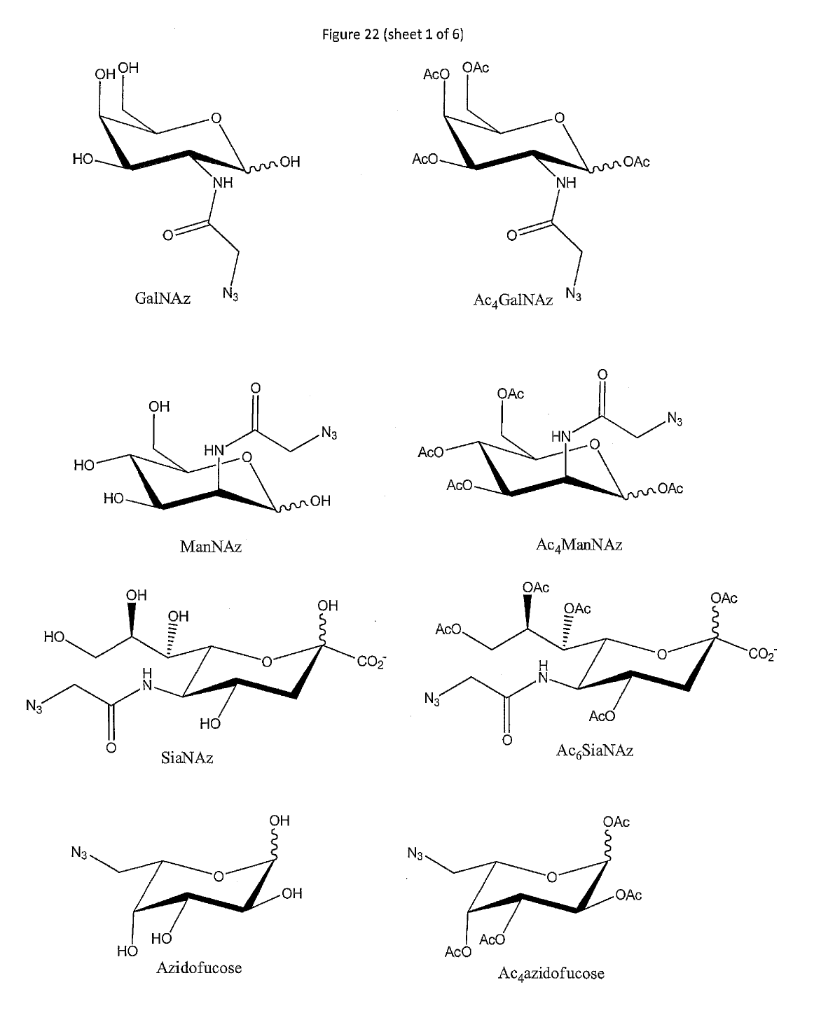

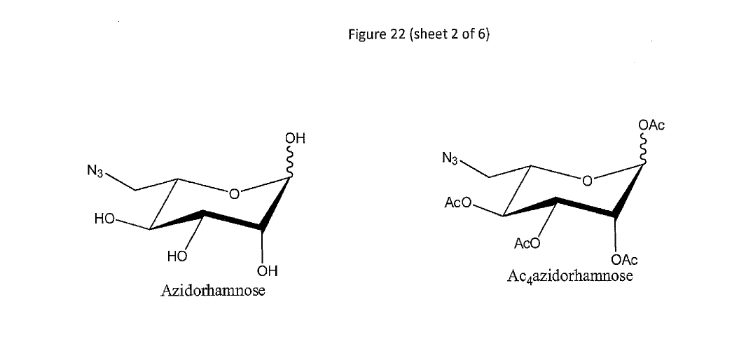

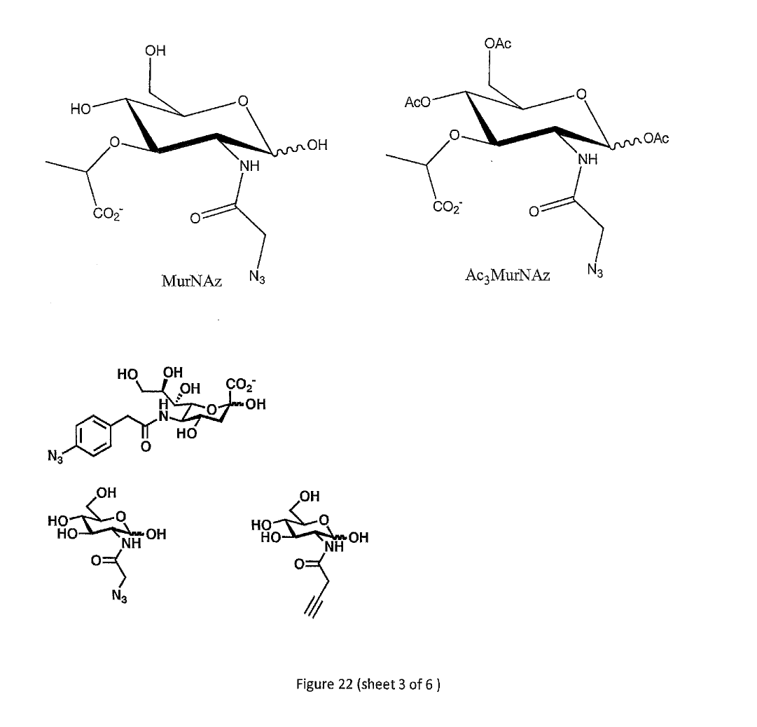

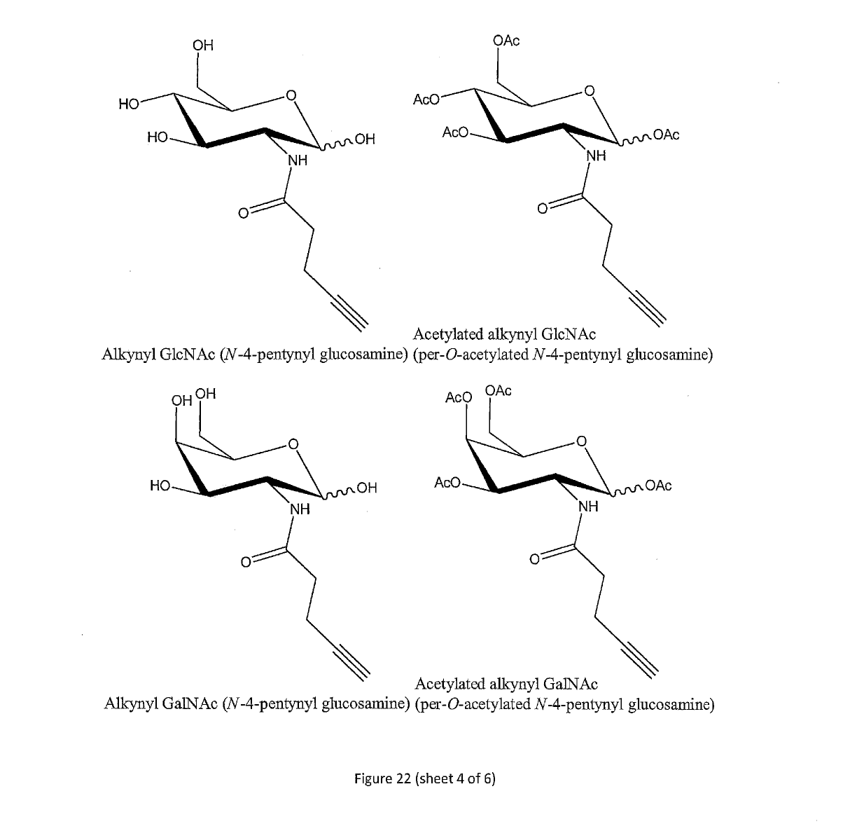

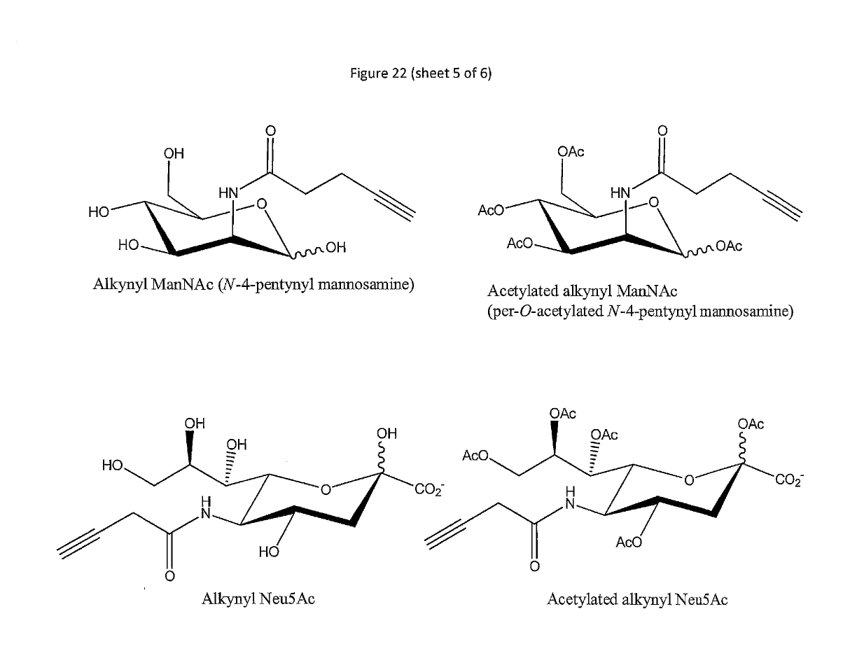

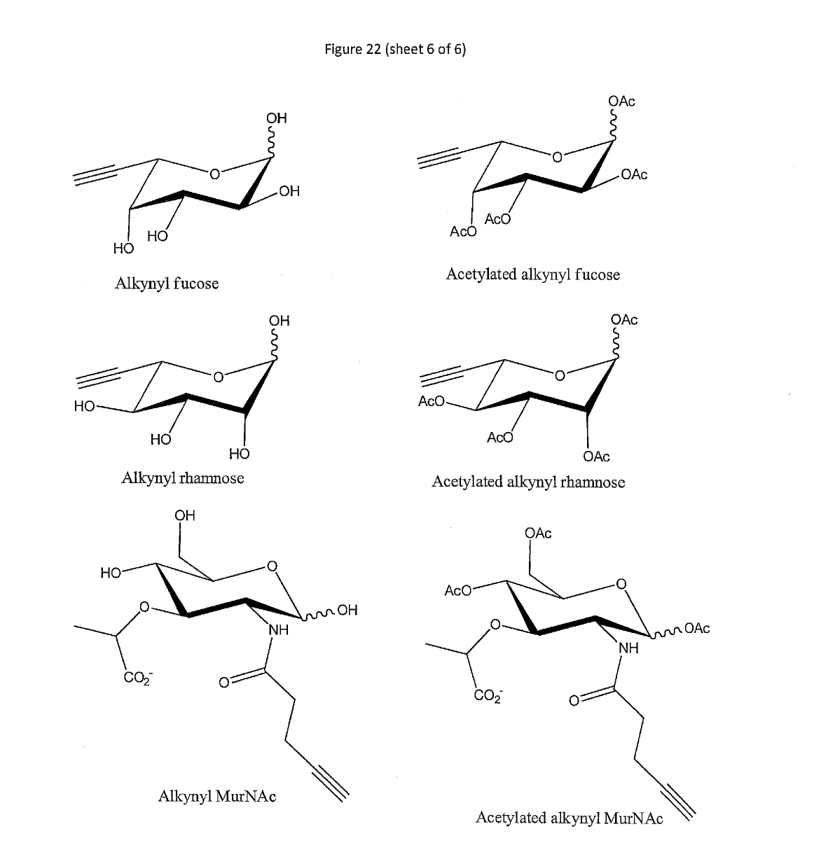

The “n” refers to the number of saccharides, and is a natural number from 1 to 10,000 such as 1, 2, 3, 4, etc. In one embodiment, the unnatural saccharide is an azido saccharide exemplified by GlcNAc, GalNAz, ManNAz, SiaNAz and their acetylated derivatives, Azidofucose and its acetylated derivatives, rhamnose (6-deoxy-L-mannose), and N-Acetylmuramic acid (FIG. 22). In a particularly preferred embodiment the azido sugar comprises GlcNAz (FIG. 22), such as O-GlcNAz. Methods for incorporating unnatural saccharide moieties into glycoproteins are described in the art (Agard et al., ACS Chem. Biol. 1, 644 (2006), Chaubard et al., Glycobiology 19, 1337 (November, 2009), Hang et al., Proceedings of the National Academy of Sciences of the United States of America 100, 14846 (Dec. 9, 2003), Luchansky et al. Biochemistry 43, 12358 (Sep. 28, 2004), Luchansky et al., Chembiochem 5, 1706 (Dec. 3, 2004), Luchansky et al., Chembiochem 5, 371 (Mar. 5, 2004), Sawa et al., Proceedings of the National Academy of Sciences of the United States of America 103, 12371 (Aug. 15, 2006), Vocadlo et al., Proceedings of the National Academy of Sciences of the United States of America 100, 9116 (Aug. 5, 2003)). Methods for conjugating the unnatural saccharide moieties with molecules of interest (e.g., fluorophores, anti-cancer agents, etc.) are known in the art (Banerjee et al., Chembiochem 11, 1273, Baskin et al., Proceedings of the National Academy of Sciences of the United States of America 107, 10360 (June), Beatty et al., Chembiochem 11, 2092 (October), Cohen et al., Journal of the American Chemical Society 132, 8563 (June), Destito et al., Chemistry & Biology 14, 1152 (2007), Hong et al., Angewandte Chemie-International Edition 48, 9879 (2009)). Additional methods for modifying the invention's unnatural saccharides are known in the art (Datta (May 22, 2002), Mahal et al., (May 16, 1997), and Yarema et al., (October, 1996)).

“Moiety” refers to a chemical functional group (i.e., a specific group of atoms) within a molecule that is responsible for the characteristic chemical reactions of those molecules. Moiety may be exemplified by amino acid moiety, saccharide moiety, chemically reactive groups such as azido group, alkyne group, and groups shown in FIG. 23.

A virus “non-structural protein” refers to a protein encoded by a virus genome, produced in infected cells, and not packaged in the mature virus particle.

Virus “structural protein” refers to a protein that is not a non-structural protein, i.e., that is encoded by a virus genome and that is packaged in the mature virus particle. Virus structural proteins include nucleocapsid core proteins (gag proteins), capsid protein, hexon protein, penton protein, fiber protein, enzymes packaged within the virus particle (pol proteins), and membrane components (env proteins).

The term “specifically binds” and “specific binding” when made in reference to the binding of two molecules (e.g., antibody to an antigen, antibody to an epitope, ligand to a receptor, transcription factor to a nucleotide sequence, etc.), refer to an interaction of the two molecules where the interaction is dependent upon the presence of particular structures on the two molecules.

The terms “reduce,” “inhibit,” “diminish,” “suppress,” “decrease,” and grammatical equivalents (including “lower,” “smaller,” etc.) when in reference to the level of any molecule (e.g., amino acid sequence, and nucleic acid sequence, antibody, etc.), cell, virus, and/or phenomenon (e.g., expression, transcription, translation, viral infection, viral productive infection, viral replication, viral replication competence, site-specific integration into a genome, viability, disease symptom, binding to a molecule, specificity of binding of two molecules, affinity of binding of two molecules, specificity to disease, sensitivity to disease, affinity of binding, enzyme activity, etc.) in a first sample (or in a first subject) relative to a second sample (or relative to a second subject), mean that the quantity of molecule, cell and/or phenomenon in the first sample (or in the first subject) is lower than in the second sample (or in the second subject) by any amount that is statistically significant using any art-accepted statistical method of analysis. In one embodiment, the quantity of molecule, cell and/or phenomenon in the first sample (or in the first subject) is at least 10% lower than, at least 25% lower than, at least 50% lower than, at least 75% lower than, and/or at least 90% lower than the quantity of the same molecule, cell and/or phenomenon in the second sample (or in the second subject). In another embodiment, the quantity of molecule, cell, and/or phenomenon in the first sample (or in the first subject) is lower by any numerical percentage from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100% lower than the quantity of the same molecule, cell and/or phenomenon in the second sample (or in the second subject). In one embodiment, the first subject is exemplified by, but not limited to, a subject that has been manipulated using the invention's compositions and/or methods. In a further embodiment, the second subject is exemplified by, but not limited to, a subject that has not been manipulated using the invention's compositions and/or methods. In an alternative embodiment, the second subject is exemplified by, but not limited to, a subject to that has been manipulated, using the invention's compositions and/or methods, at a different dosage and/or for a different duration and/or via a different route of administration compared to the first subject. In one embodiment, the first and second subjects may be the same individual, such as where the effect of different regimens (e.g., of dosages, duration, route of administration, etc.) of the invention's compositions and/or methods is sought to be determined in one individual. In another embodiment, the first and second subjects may be different individuals, such as when comparing the effect of the invention's compositions and/or methods on one individual participating in a clinical trial and another individual in a hospital.

The terms “increase,” “elevate,” “raise,” and grammatical equivalents (including “higher,” “greater,” etc.) when in reference to the level of any molecule (e.g., amino acid sequence, and nucleic acid sequence, antibody, etc.), cell, virus, and/or phenomenon (e.g., expression, transcription, translation, viral infection, viral productive infection, viral replication, viral replication competence, site-specific integration into a genome, viability, disease symptom, binding to a molecule, specificity of binding of two molecules, affinity of binding of two molecules, specificity to disease, sensitivity to disease, affinity of binding, enzyme activity, etc.) in a first sample (or in a first subject) relative to a second sample (or relative to a second subject), mean that the quantity of the molecule, cell and/or phenomenon in the first sample (or in the first subject) is higher than in the second sample (or in the second subject) by any amount that is statistically significant using any art-accepted statistical method of analysis. In one embodiment, the quantity of the molecule, cell and/or phenomenon in the first sample (or in the first subject) is at least 10% greater than, at least 25% greater than, at least 50% greater than, at least 75% greater than, and/or at least 90% greater than the quantity of the same molecule, cell and/or phenomenon in the second sample (or in the second subject). This includes, without limitation, a quantity of molecule, cell, and/or phenomenon in the first sample (or in the first subject) that is at least 10% greater than, at least 15% greater than, at least 20% greater than, at least 25% greater than, at least 30% greater than, at least 35% greater than, at least 40% greater than, at least 45% greater than, at least 50% greater than, at least 55% greater than, at least 60% greater than, at least 65% greater than, at least 70% greater than, at least 75% greater than, at least 80% greater than, at least 85% greater than, at least 90% greater than, and/or at least 95% greater than the quantity of the same molecule, cell and/or phenomenon in the second sample (or in the second subject). In one embodiment, the first subject is exemplified by, but not limited to, a subject that has been manipulated using the invention's compositions and/or methods. In a further embodiment, the second subject is exemplified by, but not limited to, a subject that has not been manipulated using the invention's compositions and/or methods. In an alternative embodiment, the second subject is exemplified by, but not limited to, a subject to that has been manipulated, using the invention's compositions and/or methods, at a different dosage and/or for a different duration and/or via a different route of administration compared to the first subject. In one embodiment, the first and second subjects may be the same individual, such as where the effect of different regimens (e.g., of dosages, duration, route of administration, etc.) of the invention's compositions and/or methods is sought to be determined in one individual. In another embodiment, the first and second subjects may be different individuals, such as when comparing the effect of the invention's compositions and/or methods on one individual participating in a clinical trial and another individual in a hospital.

The terms “alter” and “modify” when in reference to the level of any molecule and/or phenomenon refer to an increase and/or decrease.

“Substantially the same” when in reference to the level of any molecule (e.g., amino acid sequence, and nucleic acid sequence, antibody, etc.), cell, virus, and/or phenomenon (e.g., expression, transcription, translation, viral infection, viral productive infection, viral replication, viral replication competence, site-specific integration into a genome, viability, disease symptom, binding to a molecule, specificity of binding of two molecules, affinity of binding of two molecules, specificity to disease, sensitivity to disease, affinity of binding, enzyme activity, etc.) in a first sample (or in a first subject) relative to a second sample (or relative to a second subject), mean that the quantity of molecule, cell and/or phenomenon in the first sample (or in the first subject) is not different from the quantity in the second sample (or in the second subject) using any art-accepted statistical method of analysis. In one embodiment, the quantity of molecule, cell and/or phenomenon in the first sample (or in the first subject) is from 90% to 100% (e.g., 90%, 91%, 92%, 93%, 94%, 95%, 96%, 97%, 98%, 99%, and 100%) of the quantity in the second sample (or in the second subject).

Reference herein to any numerical range expressly includes each numerical value (including fractional numbers and whole numbers) encompassed by that range. To illustrate, and without limitation, reference herein to a range of “at least 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes each whole number of 5, 6, 7, 8, 9, and 10, and each fractional number such as 5.1, 5.2, 5.3, 5.4, 5.5, 5.6, 5.7, 5.8, 5.9, etc.

### DETAILED DESCRIPTION OF THE INVENTION

The invention provides compositions and methods for making and using modified viruses, including infectious viruses, having an external surface linked to at least one heterologous unnatural moiety that is exemplified by unnatural amino acid and unnatural saccharide. The unnatural moiety that is linked to the invention's modified viruses is optionally further linked to a molecule of interest (such as probe, cytotoxin, therapeutic molecule, antibody, affibody, epitope, etc.). The invention's compositions and methods find use in, for example, diagnostic applications and therapeutic applications such as gene therapy, oncolytic therapy, and/or vaccine therapy.

The invention provides the discovery that chemical modifications of viral surfaces by the selective metabolic incorporation of unnatural sugars (e.g., azidoacetylglucosamine (GlcNAz)) and/or unnatural amino acids (e.g., azidohomoalanine (AHA)) results in their site-specific display on the viral surface. These unnatural moieties serve as “chemical handles” that allow chemoselective modification in vivo using highly specific reactions that yield stable covalent products.

Coupling the flexibility of chemistry with, optionally, the specificity of genetics permits engineered viral surfaces that are amenable to labeling with highly selective “click” chemistry. The ability to remodel viruses with expanded functional properties and minimal impact on viral physiology potentially represents the next generation of highly specific, infective and/or productive viral vectors.

One advantage of the invention's compositions and methods is that genetic modification of the invention's viruses is optional, thereby simplifying and expediting generation of the viral particle.

Another advantage is that coupling the high fidelity of the biosynthetic construction of viruses with highly selective chemistry (i.e., “click” chemistry) allows increased specificity and flexibility to tailor vector properties. The resulting control over viral characteristics is advantageous for viral based diagnostics and therapeutics.

For example, data herein demonstrate that adenoviruses can be chemoselectively labeled through an exemplary two-step process. Metabolic labeling with azido sugars yields adenoviral particles with site-specific placement of a chemically accessible azide that, surprisingly, did not result in loss in either viral production or infectivity. Subsequent chemical modification of these particles allows the facile appendage of a variety of functionality from peptides to fluorophores to small molecules targeting moieties.

The invention's advantage with respect to ease and site-specificity, in combination with its advantageous non-perturbing nature with respect to viral replication and/or infectivity make it useful a wide range of applications, such as gene delivery, oncolytic viral therapy, and live vaccine design. For example, viral vectors modified by the invention's methods may be used to specifically target cancer(s), to specifically bind azide reactive immunosuppressive polymers, and to track virion dissemination in vivo.

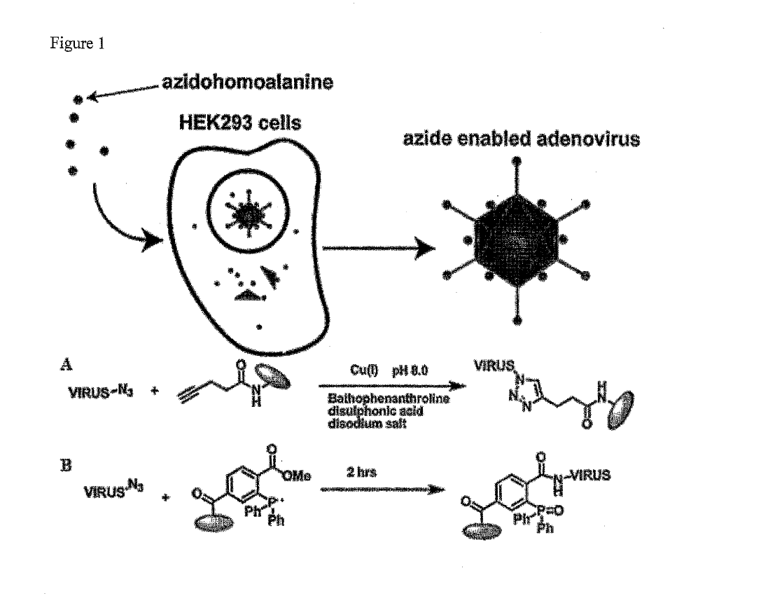

In particular, the invention provides compositions and methods for modification of viruses with unnatural amino acids. In one embodiment, data herein demonstrate an exemplary two-step labeling process involving an initial metabolic placement of the unnatural amino acid, azidohomoalanine (AHA), followed by specific chemical modification. Data herein demonstrate the introduction of a non-canonical amino acid into human adenovirus type 5 (hAd5). This process utilizes residue specific incorporation of the non-natural amino acid azidohomoalanine (AHA), a methionine surrogate, into viral capsid proteins. Surface exposure of this amino acid, AHA, allows chemical modification via the “click” reaction and Staudinger ligation (FIG. 1). Both chemistries demonstrate excellent selectivity, lead to robust linkages and allow access to virtually any type effector functionality. Surprisingly, incorporation of AHA demonstrates no impact on either particle production or infectivity. In order to explore the potential of the introduced modification sites, hAd5 particles were decorated with an exemplary cancer selective small molecule, folate. Folate-modified hAd5 conjugates demonstrated markedly increased infection of murine breast cancer cells (4T 1). This data demonstrate that incorporation of unnatural amino acids allows facile and selective chemical modification of virus vectors.

The invention also provides compositions and methods for modification of viruses with unnatural saccharides. In one embodiment, data herein demonstrate an exemplary modification of hAd5 viral surface by the metabolic incorporation of an azido sugar, O-linked N-acetylglucosamine (O-GlcNAz), into the fiber protein during virus production. Folate decorated hAd5 demonstrated a significant increase in transgene delivery to murine breast cancer cells. The data demonstrate the selective incorporation of an exemplary azido sugar, N-azidoacetylglucosamine (GlcNAz), at Ser-109 of the adenoviral fiber protein. Proximal to the primary natural targeting motif, the introduced azide allows chemoselective modification of full, infective adenovirus particles with peptides, fluorophores and small molecule targeting elements. Data herein also demonstrate that folate modification of the incorporated unnatural sugar enabled efficient gene delivery to breast cancer cells that are otherwise refractive to adenoviral infection.

For further clarity, the invention is further described under A) Modified Viruses, B) Vaccines, C) Kits, D) Exemplary Methods for Generating the Invention's Compositions, E) Exemplary Diagnostic Applications for the Invention's Compositions, and F) Exemplary Therapeutic Applications for the Invention's Compositions.

A) Modified Viruses

The invention provides modified viruses, and in particular infectious viruses, having an external surface covalently linked to at least one heterologous unnatural moiety exemplified by unnatural amino acid and unnatural saccharide. In one embodiment, the invention's viruses contain a substitution of one or more natural amino acids (e.g., Methionine) in at least one of the viral structural proteins (e.g., capsid protein, hexon protein, penton protein, and fiber protein) with one or more unnatural amino acid analogs (e.g., azidohomoalanine (AHA)) that contain a chemically reactive group (e.g., azide) that is capable of covalently binding to a molecule of interest. In another embodiment, the invention's viruses contain a substitution of one of natural saccharide (e.g., glucose) in at least one of the viral structural glycoproteins (e.g., fiber protein on the virus capsid) with an unnatural saccharide that has a chemically reactive group (e.g., azide) that is capable of covalently binding to a molecule of interest.

The invention's viruses are useful in, for example, diagnostic and therapeutic applications, such as gene delivery, oncolytic viral therapy, and vaccine design.

In particular embodiments, the invention's viruses are infectious, and more preferably, are replication competent. In a more preferred embodiment, the replication competent virus of the invention is productive. Data herein demonstrated that AHA incorporation in adenovirus has no adverse impact on the number of virus particles that were produced and on their infectivity (Examples 1-7). Data herein also demonstrate that incorporation of O-GlcNAz in adenovirus had no adverse effect on virus production and infectivity (Example 12).

While the invention is illustrated by replication competent viruses, it is not intended to be thus limited. For example, the invention also provides viruses that are not replication competent and that integrate into the genome of a cell that is susceptible to the virus. Such viruses are useful in, for example, gene replacement therapeutic applications.

In one preferred embodiments, the invention contemplates modification of one or more viral structural protein (such as Adenovirus capsid protein, hexon protein, penton protein, and fiber protein) on the virus's external surface.

The invention's viruses may be modified using click chemistry techniques. Click chemistry is a modular approach to chemical synthesis that uses a set of powerful, highly reliable, and selective reactions for the rapid synthesis of useful new compounds. Click chemistry is reviewed and described in: H. C. Kolb, K. B. Sharpless. The growing impact of click chemistry on drug discovery. Drug Discovery Today 2003, 8, 1128-1137; b) H. C. Kolb, M. G. Finn, K. B. Sharpless. Click chemistry: diverse chemical function from a few good reactions. Angew. Chem. Int. Ed. 2001, 40, 2004-2021, the contents of which are hereby incorporated by reference. A Strain promoted variant of click chemistry is also applicable to these systems. The strain promoted “click” chemistry is also very selective, reliable and rapid, however it does not depend on cytotoxic Cu(I), making it more useful in some applications. In addition, while the Staudinger reaction is, in general, slower than the “click” reactions, it also highly reliable and selective. Strain promoted click chemistry is reviewed and described in: N. J. Agard, J. M. Baskin, J. A. Prescher, A. Lo, C. R. Bertozzi, A comparative study of bioorthogonal reactions with azides ACS Chemical Biology 1, 644 (2006); N. J. Agard, J. A. Prescher, C. R. Bertozzi, A strain-promoted [3+2] azide-alkyne cycloaddition for covalent modification of biomolecules in living systems Journal of the American Chemical Society 126, 15046 (November, 2004); and K. L. Kiick, E. Saxon, D. A. Tirrell, C. R. Bertozzi, Incorporation of azides into recombinant proteins for chemoselective modification by the Staudinger ligation Proceedings of the National Academy of Sciences of the United States of America 99, 19 (January, 2002). In a particularly preferred embodiment, the viral structural protein is a viral glycoprotein (exemplified by Adenovirus fiber protein), which makes it amenable to modification with an unnatural saccharide and/or unnatural amino acid. Data herein demonstrate chemoselective modification of the human adenovirus type 5 (hAd5) viral surface by the metabolic incorporation of the unnatural saccharide (azido sugar) O-linked N-acetylglucosamine (O-GlcNAz) into the fiber protein during virus production and the chemoselective attachment of an exemplary cancer selective small molecule, folate, to the O-GlcNAz-modified viral surface (Examples 10-15).

The invention is illustrated herein by substitution of a natural moiety with an unnatural moiety. Data herein demonstrates the exemplary substitution of the natural monosaccharide O-GlcNAc residue on Ser-109 of the adenovirus fiber protein with the unnatural monosaccharide azido analog O-GlcNAz (Example 9-14). Further data herein also demonstrate the exemplary substitution of the natural amino acid methionine of the adenovirus capsid protein with the unnatural amino acid azidohomoalanine (AHA) (Examples 1-7).

However, the invention is not limited to substitution, but also contemplates that the unnatural moiety is inserted (i.e., added) into the external surface of the virus. Methods for insertion of an unnatural moiety are described in Connor et al., Chembiochem 9, 366 (Jan. 15, 2008).

While not intending to limit the methodology of inserting and/or adding the unnatural moiety into the external surface of the virus, in a preferred embodiment, the linkage of the unnatural moiety and the virus external surface is metabolically produced instead of being recombinantly produced and/or chemically produced.

It is contemplated that the unnatural moiety (e.g., unnatural amino acid and/or unnatural saccharide) used for modifying the viruses of the invention contain one or more chemically reactive group that is capable of covalently binding to a molecule of interest. Chemically reactive groups are exemplified by the azido group, alkyne group, and groups shown in FIG. 23.

1) Viruses

While the invention has been exemplified using adenoviral vectors, it is not limited to adenoviruses, but is contemplated to be useful with a wide variety of viruses (e.g., including those having peripheral proteins and/or glycoproteins that are suitable for modification with unnatural sugars and/or unnatural amino acids), including retrovirus, herpes simplex virus, poxvirus, adeno-associated virus, baculovirus, measles virus, lentivirus, oncovirus, hybrid virus, and recombinant virus. In particular, the invention's compositions and methods are useful with oncolytic viruses (e.g., retroviruses, lentiviruses, poxviruses and herpes viruses).

In a particular embodiment, the virus genome sequence is gutted. The term “gutted” and “fully deleted” are used interchangeably in reference to a viral vector, and refer to a viral vector (e.g., naked DNA, plasmid, virus particle, etc.) that lacks all the coding sequences that are otherwise present in a wild type virus. Gutted vectors may contain non-coding viral sequences, e.g., terminal repeat sequences, and packaging sequences. For example, a gutted adenovirus vector lacks all adenovirus coding sequences and optionally contains adenovirus terminal repeat sequences and/or packaging sequences (e.g., U.S. Pat. No. 5,994,132 to Chamberlain et al., U.S. Pat. No. 6,797,265 to Amalfitano et al., U.S. Pat. No. 7,563,617 to Hearing et al., and U.S. Pat. No. 6,262,035 to Campbell et al.). Gutted vectors are preferred in certain embodiments since they do not express viral vector proteins and hence do not induce an adverse immune or toxic response in a cell.

i) Adenovirus

Thus in a particular embodiment, the invention's virus (and/or hybrid virus and/or recombinant virus) comprises at least a portion of adenovirus. “Adenovirus” refers to a double-stranded DNA virus with a genome of approximately 36 Kb flanked by inverted terminal repeats. Adenovirus boasts of a wide tropism which can be increased by replacement of the fiber knob carried on the icosahedral capsid, responsible for contact with the host receptor, with that of another serotype. Adenovirus is of animal origin, such as avian, bovine, ovine, murine, porcine, canine, simian, and human origin. Avian adenoviruses are exemplified by serotypes 1 to 10 that are available from the ATCC, such as, for example, the Phelps (ATCC VR 432), Fontes (ATCC VR 280), P7 A (ATCC VR 827), IBH 2A (ATCC VR 828), J2 A (ATCC VR 829), T8 A (ATCC VR 830), and K 11 (ATCC VR 921) strains, or else the strains designated as ATCC VR 831 to 835. Bovine adenoviruses are illustrated by those available from the ATCC (types 1 to 8) under reference numbers ATCC VR 313, 314, 639 642, 768 and 769. Ovine adenoviruses include the type 5 (ATCC VR 1343) or type 6 (ATCC VR 1340). Murine adenoviruses are exemplified by FL (ATCC VR 550) and E20308 (ATCC VR 528). Porcine adenovirus (5359) may also be used. adenoviruses of canine origin include all the strains of the CAVI and CAV2 adenoviruses (for example, Manhattan strain or A26/61 (ATCC VR 800) strain). Simian adenoviruses are also contemplated, and they include the adenoviruses with the ATCC reference numbers VR 591 594, 941 943, and 195 203. Human adenoviruses, of which there greater than fifty (50) serotypes are known in the art, are also contemplated, including Ad2, Ad3, Ad4, Ad5, Ad11, Ad14, Ad7, Ad9, Ad12, Ad16, Ad17, Ad21, Ad26, Ad34, Ad35, Ad 40, Ad48, Ad49, Ad50 (e.g., U.S. Pat. No. 7,300,657 to Pau, U.S. Pat. No. 7,468,181 to Vogels, and U.S. Pat. No. 6,136,594 to Dalemans). In one preferred embodiment, the adenovirus is selected from adenovirus 2 (Ad2) and adenovirus 5 (Ad5).

Adenoviruses of animal origin can be obtained, for example, from strains deposited in collections, then amplified in competent cell lines and modified as required (Hearing et al., U.S. Pat. No. 7,563,617). Techniques for producing, isolating and modifying adenoviruses have been described in the literature and may be used within the scope of the present invention (Akli et al., Nature Genetics 3 (1993) 224; Stratford-Perricaudet et al., Human Gene Therapy 1 (1990) 241; patent EP 185 573, Levrero et al., Gene 101 (1991) 195; Le Gal la Salle et al., Science 259 (1993) 988; Roemer and Friedmann, Eur. J. Biochem. 208 (1992) 211; Dobson et al., Neuron 5 (1990) 353; Chiocca et al., New Biol. 2 (1990) 739; Miyanohara et al., New Biol. 4 (1992) 238; WO 91/18088, WO 90/09441, WO 88/10311, WO 91/11525). These different viruses can then be modified, for example, by deletion, substitution, addition, etc. The complete genome sequences have been determined for human adenovirus type 2 (GenBank Accession No. J01917), human adenovirus type 5 (GenBank Accession No. M73260; and GenBank Accession No. NC-001406), human adenovirus type 12 (GenBank Accession No. NC-001460, X73487); human adenovirus type 17 (GenBank Accession No. NC-002067, AF108105), and human adenovirus type 40 (GenBank Accession No. L19443).

Adenovirus vectors have been used in gene therapy, particularly cancer therapy. e.g., vector ONYX015 (Heise C (1997) Nature Med. 3:639-645; Rothmann et al. (1998) J. Virol. 72:9470).

Adenovirus vectors have also been used as Ad-based vaccines for multiple diseases including Tuberculosis (Magalhaes et al. (2008) PLoS ONE 3:e3790), malaria (Shott et al. (2008) Vaccine 26:2818-2823), rabies (Zhou et al. (2006) Mol Ther 14:662-672), influenza (Hoelscher et al. (2008) J Infect Dis 197:1185-1188), and leishmania (Resende et al. (2008) Vaccine 26:4585-4593).

In a more particular embodiment, the adenovirus lacks at least a portion of one or more adenovirus early gene region

“Adenovirus early gene regions” refers to nucleotide sequences which are derived from adenovirus and which are transcribed prior to replication of the adenovirus genome. The early gene regions comprise E1a, E1b, E2a, E2b, E3 and E4. The E1a gene products are involved in transcriptional regulation; the E1b gene products are involved in the shut-off of host cell functions, mRNA transport, regulation of apoptosis induction, and inhibition of p53 tumor suppressor. E2a encodes a DNA-binding protein (DBP); E2b encodes the viral DNA polymerase and preterminal protein (pTP). The E3 gene products are not essential for viral growth in cell culture. The E4 regions encode regulatory proteins involved in transcriptional and post-transcriptional regulation of viral gene expression; a subset of the E4 proteins are essential for viral growth. In contrast to the adenovirus early gene regions, the “adenovirus late gene regions” refers to adenovirus nucleotide sequences that are transcribed after replication. The products of the late genes (e.g., L1-5) are predominantly components of the virion as well as proteins involved in the assembly of virions. The VA genes produce VA RNAs that block the host cell from shutting down viral protein synthesis. The early and late gene regions of adenovirus have been characterized (e.g., in Ad2 genomic sequence; GenBank No. J01917). Thus, in particular embodiments, the adenovirus lacks adenovirus E1 gene coding sequence and/or lacks adenovirus E3 gene coding sequence.

ii) Retrovirus

In another particular embodiment, the invention's virus (and/or hybrid virus and/or recombinant virus) comprises at least a portion of a retrovirus. “Retrovirus” is a small enveloped RNA virus, containing two identical single stranded positive sense RNA genomes enclosed in an enveloped capsid. Retroviruses have a genome flanked by Long Terminal Repeats (LTR) and 4 main genes gag, pol, pro and env.

Retroviruses include, but are not limited to the following genera: Genus Lentivirus (e.g., Human immunodeficiency virus; Simian immunodeficiency virus, Feline immunodeficiency virus), Genus Alpharetrovirus (e.g., Avian leukosis virus, Rous sarcoma virus), Genus Betaretrovirus (e.g., Mouse mammary tumor virus), Genus Gammaretrovirus (e.g., Murine leukemia virus, Feline leukemia virus), Genus Deltaretrovirus (e.g., Bovine leukemia virus, cancer-causing Human T-lymphotropic virus), Genus Epsilonretrovirus (e.g., Walleye dermal sarcoma virus), and Genus Spumavirus (e.g., Simian foamy virus).

In a particular embodiment, the invention's virus (and/or hybrid virus and/or recombinant virus) comprises at least a portion of a lentivirus. “Lentiviruses” are a group of complex retroviruses that carry accessory gene which regulate and coordinate viral gene expression. Lentiviruses also differ from other retroviruses in their ability to infect non-dividing cells as most other retroviruses are incapable of traversing the nuclear membrane and can thus infect only dividing cells where the nuclear membrane is dissolved.

iii) Herpes Simplex Virus

Also, in a particular embodiment, the invention's virus (and/or hybrid virus and/or recombinant virus) comprises at least a portion of a herpes simplex virus, such as, without limitation, HSV-1 and HSV-2. “Herpes simplex virus” also referred to as “HSV”, is an enveloped virus with a linear double stranded DNA (dsDNA) genome of 152 Kb, carrying 74 separate genes. The genome consists of 2 unique sequences, one longer than the other (U_{L} and U_{S}). Each of these sequences are flanked by inverted terminal repeat sequences—with U_{L} flanked by Terminal Repeat (TR_{L}) and Internal Repeat (IR_{L}) and U_{S} being flanked by IR_{S} and TR_{S}. Copies of an ‘a’ sequence carrying packaging signals lie between the two IRs and at each TR. HSV is exemplified by HSV-1 and HSV-2, which are neurotropic pathogens associated with a number of skin diseases from herpes labialis and herpes genitalis to the life threatening neonatal herpes and herpes encephalitis (Watanabe D (2010) Journal of Dermatological Science 57(2):75-82).

Generic methods are known for producing oncolytic HSV based vectors and DNA vaccines. The two main types of HSV based vectors used are amplicon vectors and replication attenuated vectors.

Amplicon vectors are plasmids made up of repeated units of the transgene, a packaging signal (pac) and an HSV origin of replication (R.R. S & N. F (1982) Cell 30:295). When introduced into a cell along with HSV helper functions, these amplicons replicate and are packaged as head to tail concatemers into infectious HSV virions. HSV Amplicons have been used as DNA vaccines (Santos et al. (2006) Curr Gene Ther 6(3):383-392).

Replication attenuated HSV vectors have been used as oncolytic vectors. These vectors have deletions in genes (such as HSV-TK and HSV-RR) that are required for replication of the virus in non-dividing cells and are thus capable of replication only in dividing (tumor) cells. Clinical trials (Phase I) for multiple HSV-1 derived oncolytic viruses for colorectal carcinoma (Kemeny N, et al. (2006) Hum Gene Ther 17(12):1214-1224), melanoma (MacKie et al. (2001) Lancet 357(9255):525-526), breast cancer (Hu et al. (2006) Clin Cancer Res 12(22):6737-6747), and malignant glioma (Markert et al. (2000) Gene Ther 7(10):867-874), among others have been reported. All studies reported safety and toleration of HSV vectors. For example, herpes simplex virus named OncoVEX GM-CSF is in Phase 3 clinical trials in melanoma and head and neck cancer.

iv) Poxvirus

In one embodiment, the invention's virus (and/or hybrid virus and/or recombinant virus) comprises at least a portion of a poxvirus.

v) AAV

In one embodiment, the invention's virus (and/or hybrid virus and/or recombinant virus) comprises at least a portion of adeno-associated virus (AAV). AAV is widely used as gene transfer vehicles today, capable of long term extra chromosomal persistence in several tissues. Prior art production of rAAV requires AAV ITR flanked transgene, AAV Rep and Cap genes and helper virus. Prior art rAAV do not carry Rep due to size constraints and worries about toxicity and are therefore incapable of site-specific integration. The only AAV elements retained in prior art rAAV are the AAV ITRs which flank the transgene of interest. Currently, prior art production of rAAV requires co-transfection of multiple plasmid constructs, bearing the AAV ITR flanked transgene construct, the AAV Rep-Cap coding sequences and the Adenovirus helper functions including E2, E4 and VA into cell lines such as 293 which provide Ad E1 functions or infection of producer cell lines carrying an integrated Rep Cap cassette and the rAAV sequence, with a helper Ad.

The FDA has approved clinical trial, for rAAV vectors expressing the Cystic Fibrosis Transmembrane Conductance Regulator (rAAV2-CFTR) (Flotte (1996) Hum. Gene Ther. 7:1145-1159; Flotte et al. (2003) Hum Gene Ther 14(11):1079-1088), rAAV2-FIX for the delivery of coagulation FIX to patients with Hemophilia B (Manno et al. (2003) Blood 101(8):2963-2972; Manno et al. (2006) Nat Med 12(3):342-347), rAAV2-hRPE65v2 vectors for expression of RPE65 in the treatment of Leber's congenital Amaurosis (LCA) (Bennicelli et al. (2008) Mol Ther 16(3):458-465), rAAV vector CERE-20 expressing the neurotrophic factor Neurturin (NTN) to protect against the degeneration of dopaminergic neurons associated with Parkinson's disease, and rAAV2 vector expressing al antitrypsin for al antitrypsin (AAT) deficiency associated lung disease (Brantly et al. (2009) PNAS 106(38):16363-16368; Mingozzi et al. (2009) Blood 114(10):2077-2086).

Methods for production of rAAV vectors and AAV containing these vectors are known in the art (Carter, U.S. Pat. No. 7,785,888). For example, methods for achieving high titers of rAAV virus preparations that are substantially free of contaminating virus and/or viral or cellular proteins are outlined by Atkinson et al. in WO 99/11764. Techniques described therein can be employed for the large-scale production of rAAV viral particle preparations (Carter, U.S. Pat. No. 7,785,888).

These various examples address the production of rAAV viral particles at sufficiently high titer, minimizing recombination between rAAV vector and sequences encoding packaging components, and producing rAAV virus preparations that are substantially free of contaminating virus and/or viral or cellular protein (Carter, U.S. Pat. No. 7,785,888).

Optionally, rAAV virus preparations can be further processed to purify (i.e., enrich for) rAAV particles and/or otherwise render them suitable for administration to a subject. See Atkinson et al. for exemplary techniques (WO 99/11764). Purification techniques can include isopynic gradient centrifugation, and chromatographic techniques. Reduction of infectious helper virus activity can include inactivation by heat treatment or by pH treatment as is known in the art. Other processes can include concentration, filtration, diafiltration, or mixing with a suitable buffer or pharmaceutical excipient. Preparations can be divided into unit dose and multi dose aliquots for distribution, which will retain the essential characteristics of the batch, such as the homogeneity of antigenic and genetic content, and the relative proportion of contaminating helper virus (Carter, U.S. Pat. No. 7,785,888).

Various methods for the determination of the infectious titer of a viral preparation are known in the art. For example, one method for titer determination is a high-throughput titering assay as provided-by Atkinson et al. (WO 99/11764). Virus titers determined by this rapid and quantitative method closely correspond to the titers determined by more classical techniques. In addition, however, this high-throughput method allows for the concurrent processing and analysis of many viral replication reactions and thus has many others uses, including for example the screening of cell lines permissive or non-permissive for viral replication and infectivity (Carter, U.S. Pat. No. 7,785,888).

vi) Baculovirus

In another embodiment, the invention's virus (and/or hybrid virus and/or recombinant virus) comprises at least a portion of a baculovirus. “Baculoviruses” are a family of large rod-shaped viruses that can be divided to two genera: nucleopolyhedroviruses (NPV) and granuloviruses (GV). While GVs contain only one nucleocapsid per envelope, NPVs contain either single (SNPV) or multiple (MNPV) nucleocapsids per envelope. The enveloped virions are further occluded in granulin matrix in GVs and polyhedrin for NPVs. Moreover, GV have only single virion per granulin occlusion body while polyhedra contains multiple embedded virions. Baculoviruses have very species-specific tropisms among the invertebrates with over 600 host species having been described. They are not known to replicate in mammalian or other vertebrate animal cells. Baculoviruses contain circular double-stranded genome ranging from 80-180 kbp.

Baculovirus expression in insect cells represents a robust method for producing recombinant glycoproteins. Baculovirus-produced proteins have several immunologic advantages over proteins derived from mammalian sources and are attractive candidates for therapeutic cancer vaccines (Betting et al., Enhanced immune stimulation by a therapeutic lymphoma tumor antigen vaccine produced in insect cells involves mannose receptor targeting to antigen presenting cells. Vaccine. 2009 Jan. 7; 27(2):250-9. Epub 2008 Nov. 8. PMID: 19000731).

vii) Measles Virus

In a further embodiment, the invention's virus (and/or hybrid virus and/or recombinant virus) comprises at least a portion of measles virus (e.g., measles virus echistatin vector (MV-ERV), Hallak et al., Cancer Res Jun. 15, 2005 65; 5292).

viii) Oncovirus

In another embodiment, the invention's virus (and/or hybrid virus and/or recombinant virus) comprises at least a portion of an oncovirus. “Oncovirus” and “oncolytic virus” interchangeably refer to a virus that specifically and/or preferentially infects and lyses cancer cells compared to normal cells. Oncoviruses may be used in cancer therapy, both for direct destruction of the cancer cells, and as vectors enabling genes expressing anticancer molecules to be delivered specifically to cancer cells.

Oncolytic viruses are exemplified by adenovirus (e.g., ONYX-015 virus in which the E1B-55 kDa gene has been deleted allowing the virus to selectively replicate in and lyse p53-deficient cancer cells), measles virus (e.g., measles virus echistatin vector (MV-ERV), Hallak et al., Cancer Res Jun. 15, 2005 65; 5292), herpes simplex virus (e.g., OncoVEX GM-CSF virus which is in Phase 3 clinical trials in melanoma and head and neck cancer).

Cancer cell specificity of oncolytic viruses may be achieved by art-known transduction and non-transductional targeting. Transductional targeting (e.g., using bi-specific adapter fusion proteins & modifying virus coat proteins) involves modifying the specificity of viral coat protein, thus increasing entry into target cells while reducing entry to non-target cells. Non-transductional targeting (e.g., transcriptional targeting by plasin an essential viral gene under control of a tumor-specific promoter, attenuation by deleting viral gene regions to eliminate viral functions that are expendable in cancer cell, but not in normal cells) involves altering the genome of the virus so it can only replicate in cancer cells. This can be done by either transcription targeting, where genes essential for viral replication are placed under the control of a cancer-specific promoter, or by attenuation, which involves introducing deletions into the viral genome that eliminate functions that are dispensable in cancer cells, but not in normal cells.

2) Molecule of Interest

In preferred embodiments the unnatural moiety, that is covalently linked to the external surface of the invention's modified viruses, is also covalently linked to molecule of interest, exemplified by probe, cytotoxin, therapeutic molecule, antibody, affibody, epitope, etc.

i) Probes

In one embodiment, the molecule of interest comprises a probe. Viruses thus modified are useful in, for example, detecting disease in diagnostic applications and/or detecting viruses labeled with the probes.

“Probe” “reporter” and “label” are interchangeably used to describe a chemical moiety that, when attached to a composition of interest, acts as a marker for the presence of the composition of interest, such that detection of the label corresponds to detection of the composition of interest. Probes include fluorescent and chemiluminescent compound (e.g., fluorescein isothiocyanate, rhodamine, and/or luciferin) and/or an enzyme (e.g., alkaline phosphatase, beta-galactosidase and/or horseradish peroxidase). In some embodiments, the probe comprises a fluorophore (e.g., Phosphine-FLAG, Phosphine-FLAGₕᵢₛ, Alk-TAMRA). In another embodiment, the probe comprises biotin acceptor peptide and/or biotin. Labeling reagents and kits are commercially available to detect proteins and/or glycoproteins that are labeled with biotin.

ii) Cytotoxins

In another embodiment, the molecule of interest comprises a cytotoxin. Viruses thus modified are useful in, for example, treatment of disease (e.g., cancer). In some embodiments, the cytotoxin comprises an anti-cancer toxin.

“Cytotoxic” molecule refers any molecule that reduces proliferation and/or viability of a target cell, preferably, though not necessarily, killing the target cell. In a preferred embodiment, the cytotoxic molecule is an anti-cancer toxin.

“Anti-cancer toxin” and “anti-cancer cytotoxin” is a molecule that reduce proliferation and/or viability of cancer cells. In preferred embodiments, anti-cancer toxins delay the onset of development of tumor development and/or reduce the number, weight, volume, and/or growth rate of tumors. Cytotoxins are exemplified by, without limitation, second messengers such as cAMP; Bacterial toxins such as the exemplary Pertussis toxin, Cholera toxin, and C3 exoenzyme; Lectins such as Ricin A (Engert et al. Blood. 1997 Jan. 15; 89(2):403-10.). Also included are toxins exemplified by Topoisomerase inhibitors such as etoposide, Campothecin irinotecan, topotecan, anthracyclines (doxorubicine, daunorubicine); Microtubule inhibitors such as vincristine, vinblastine, vinorelbine, paclitaxel, docetaxel; Platinum containing compounds such as cisplatin, carboplatin, oxaloplatin, etc.; Alkylating agents such as cyclophosphamide, and ifosfamide; Antimetabolites such as methotrexate and mercaptoprine; Anti-estrogens such as tamoxifen and toremifene; Retinoids such as all trans-retinoic acid; and others such as Adriamycin, gemcitabine, and 5-fluoruracil.

A number of the above-mentioned toxins also have a wide variety of analogues and derivatives, including, but not limited to, cisplatin, cyclophosphamide, misonidazole, tiripazamine, nitrosourea, mercaptopurine, methotrexate, fluorouracil, epirubicin, doxorubicin, vindesine and etoposide. Analogues and derivatives include (CPA).sub.2Pt(DOLYM) and (DACH)Pt(DOLYM) cisplatin, Cis-(PtCl.sub.2(4,7-H-5-methyl-7-oxo-)1,2,4(triazolo(1,5-a)pyrimidine).sub.2), (Pt(cis-1,4-DACH)(trans-Cl.sub.2)(CBDCA)).multidot.-1/2MeOH cisplatin, 4-pyridoxate diammine hydroxy platinum, Pt(II).Pt(II) (Pt.sub.2(NHCHN(C(CH.sub.2)(CH.s-ub.3))).sub.4), 254-S cisplatin analogue, O-phenylenediamine ligand bearing cisplatin analogues, trans, cis-(Pt(OAc).sub.21.sub.2(en)), estrogenic 1,2-diarylethylenediamine ligand (with sulfur-containing amino acids and glutathione) bearing cisplatin analogues, cis-1,4-diaminocyclohexane cisplatin analogues, 5′ orientational isomer of cis-(Pt(NH.sub.3)(4-aminoTEMP-O){d(GpG)}), chelating diamine-bearing cisplatin analogues, 1,2-diarylethyleneamine ligand-bearing cisplatin analogues, (ethylenediamine)platinum-(II) complexes, CI-973 cisplatin analogue, cis-diamminedichloroplatinum(II) and its analogues cis-1,1-cyclobutanedicarbosylato(2R)-2-methyl-1,4-butanediam-mineplatinum-(II) and cis-diammine(glycolato)platinum, cis-amine-cyclohexylamine-dichloroplatinum(II), gem-diphosphonate cisplatin analogues (FR 2683529), (meso-1,2-bis(2,6-dichloro-4-hydroxyplenyl)ethylenediamine)dichloroplatinum(II), cisplatin analogues containing a tethered dansyl group, platinum(II)polyamines, cis-(3H)dichloro(ethylenediamine)platinu-m(II), trans-diamminedichloroplatinum(II) and cis-(Pt(NH.sub.3).sub.2(N.sub.3-cy-tosine)Cl), 3H-cis-1,2-diaminocyclohexanedichloroplatinum(II) and 3H-cis-1,2-diaminocyclohexane-malonatoplatinum (II), diaminocarboxylatoplatinum (EPA 296321), trans-(D,1)-1,2-diaminocyclohexa-ne carrier ligand-bearing platinum analogues, aminoalkylaminoanthraquinone-deri-ved cisplatin analogues, spiroplatin, carboplatin, iproplatin and JM40 platinum analogues, bidentate tertiary diamine-containing cisplatinum derivatives, platinum(II), platinum(IV), cis-diammine (1,1-cyclobutanedicarboxylato-)platinum(II) (carboplatin, JM8) and ethylenediammine-malonatoplatinum(II) (JM40), JM8 and JM9 cisplatin analogues, (NPr4)2((PtCL4).cis-(PtC12-(NH2Me)₂)), aliphatic tricarboxylic acid platinum complexes (EPA 185225), cis-dichloro(amino acid) (tert-butylamine)platinum-(II) complexes; 4-hydroperoxycylcophosphamide, acyclouridine cyclophosphamide derivatives, 1,3,2-dioxa- and -oxazaphosphorinane cyclophosphamide analogues, C5-substituted cyclophosphamide analogues, tetrahydrooxazine cyclophosphamide analogues, phenyl ketone cyclophosphamide analogues, phenylketophosphamide cyclophosphamide analogues, ASTA Z-7557 cyclophosphamide analogues, 3-(1-oxy-2,2,6,6-tetramethyl-4-piperidinyl)cy-clophosphamide, 2-oxobis(2-β-chloroethylamino)-4-, 6-dimethyl-1,3,2-oxazaphosphorinan-e cyclophosphamide, 5-fluoro- and 5-chlorocyclophosphamide, cis- and trans-4-phenylcyclophosphamide, 5-bromocyclophosphamide, 3,5-dehydrocyclophosphamide, 4-ethoxycarbonyl cyclophosphamide analogues, arylaminotetrahydro-2H-1,3,2-oxazaphosphorine 2-oxide cyclophosphamide analogues, NSC-26271 cyclophosphamide analogues, benzo annulated cyclophosphamide analogues, 6-trifluoromethylcyclophosphamide, 4-methylcyclophosphamide and 6-methycyclophosphamide analogues; FCE 23762 doxorubicin derivative, annamycin, ruboxyl, anthracycline disaccharide doxorubicin analogue, N-(trifluoroacetyl)doxorubicin and 4′-O-acetyl-N-(trifluoroacetyl)-doxorubicin, 2-pyrrolinodoxorubicin, disaccharide doxorubicin analogues, 4-demethoxy-7-O-(2,6-dideoxy-4-O-(2,3,6-trideoxy-3-amino-α-L-lyxo-hexopyranosyl)-α-L-lyxo-hexopyranosyl)adriamicinone doxorubicin disaccharide analog, 2-pyrrolinodoxorubicin, morpholinyl doxorubicin analogues, enaminomalonyl-β-alanine doxorubicin derivatives, cephalosporin doxorubicin derivatives, hydroxyrubicin, methoxymorpholino doxorubicin derivative, (6-maleimidocaproyl)hydrazone doxorubicin derivative, N-(5,5-diacetoxypent-1-yl)doxorubicin, FCE 23762 methoxymorpholinyl doxorubicin derivative, N-hydroxysuccinimide ester doxorubicin derivatives, polydeoxynucleotide doxorubicin derivatives, morpholinyl doxorubicin derivatives (EPA 434960), mitoxantrone doxorubicin analogue, AD 198 doxorubicin analogue, 4-demethoxy-3′-N-trifluoroacetyldoxorubicin, 4′-epidoxorubicin, alkylating cyanomorpholino doxorubicin derivative, deoxydihydroiodooxorubicin (EPA 275966), adriblastin, 4′-deoxydoxorubicin, 4-demethyoxy-4′-o-methyldoxorubicin, 3′-deamino-3′-hydroxydoxorubicin, 4-demethyoxy doxorubicin analogues, N-L-leucyl doxorubicin derivatives, 3′-deamino-3′-(4-methoxy-1-piperidinyl)doxorubicin derivatives (U.S. Pat. No. 4,314,054), 3′-deamino-3′-(4-mortholinyl)doxorubicin derivatives (U.S. Pat. No. 4,301,277), 4′-deoxydoxorubicin and 4′-o-methyldoxorubicin, aglycone doxorubicin derivatives, SM 5887, MX-2,4′-deoxy-13(S)-dihydro-4′-iododoxorubicin (EP 275966), morpholinyl doxorubicin derivatives (EPA 434960), 3′-deamino-3′-(4-methoxy-1-piperidi-nyl)doxorubicin derivatives (U.S. Pat. No. 4,314,054), doxorubicin-14-valerate, morpholinodoxorubicin (U.S. Pat. No. 5,004,606), 3′-deamino-3′-(3′-cyano-4″-morpholinyl doxorubicin; 3′-deamino-3′-(3″-cyano-4″-morpholinyl)-13-dihydroxorubicin; (3′-deamino-3′-(3″-cyano-4″-morpholinyl)daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl)doxorubicin derivatives (U.S. Pat. No. 4,314,054) and 3-deamino-3-(4-morpholinyl)doxorubicin derivatives (U.S. Pat. No. 4,301,277); 4,5-dimethylmisonidazole, azo and azoxy misonidazole derivatives; RB90740; 6-bromo and 6-chloro-2,3-dihydro-1,4-benzothi-azines nitrosourea derivatives, diamino acid nitrosourea derivatives, amino acid nitrosourea derivatives, 3′,4′-didemethoxy-3′,4′-dio-xo-4-deoxypodophyllotoxin nitrosourea derivatives, ACNU, tertiary phosphine oxide nitrosourea derivatives, sulfamerizine and sulfamethizole nitrosourea derivatives, thymidine nitrosourea analogues, 1,3-bis(2-chloroethyl)-1-nitrosourea, 2,2,6,6-tetramethyl-1-oxopiperidiunium nitrosourea derivatives (U.S.S.R. 1261253), 2- and 4-deoxy sugar nitrosourea derivatives (U.S. Pat. No. 4,902,791), nitroxyl nitrosourea derivatives (U.S.S.R. 1336489), fotemustine, pyrimidine (II) nitrosourea derivatives, CGP 6809, B-3839, 5-halogenocytosine nitrosourea derivatives, 1-(2-chloroethyl)-3-isobu-tyl-3-(β-maltosyl)-1-nitrosourea, sulfur-containing nitrosoureas, sucrose, 6-((((2-chloroethyl)nitrosoamino-)carbonyl)amino)-6-deoxysucrose (NS-1C) and 6′-((((2-chloroethyl)nitrosoamino)carbonyl)amino)-6′-deoxysucrose (NS-1D) nitrosourea derivatives, CNCC, RFCNU and chlorozotocin, CNUA, 1-(2-chloroethyl)-3-isobutyl-3-(β-maltosyl)-1-nitrosourea, choline-like nitrosoalkylureas, sucrose nitrosourea derivatives (JP 84219300), sulfa drug nitrosourea analogues, DONU, N,N′-bis(N-(2-chloroethyl)-N-nitrosocarbamoyl)cystamine (CNCC), dimethylnitrosourea, GANU, CCNU, 5-aminomethyl-2′-deoxyuridine nitrosourea analogues, TA-077, gentianose nitrosourea derivatives (JP 82 80396), CNCC, RFCNU, RPCNU AND chlorozotocin (CZT), thiocolchicine nitrosourea analogues, 2-chloroethyl-nitrosourea, ACNU, (1-(4-amino-2-methyl-5-pyrimidinyl)methyl-3-(2-chloroethyl)-3-nitrosourea hydrochloride), N-deacetylmethyl thiocolchicine nitrosourea analogues, pyridine and piperidine nitrosourea derivatives, methyl-CCNU, phensuzimide nitrosourea derivatives, ergoline nitrosourea derivatives, glucopyranose nitrosourea derivatives (JP 78 95917), 1-(2-chloroethyl)-3-cyclohexyl-1-nitrosourea, 4-(3-(2-chloroethyl)-3-nitrosoureid-o)-cis-cyclohexanecarboxylic acid, RPCNU (ICIG 1163), IOB-252, 1,3-bis(2-chloroethyl)-1-nitrosourea (BCNU), 1-tetrahydroxycyclopentyl-3-nitroso-3-(2-chloroethyl)-urea (4,039,578), d-1-1-(β-chloroethyl)-3-(2-oxo-3-hexahydroazepinyl)-1-nitrosourea (3,859,277) and gentianose nitrosourea derivatives (JP 57080396); 6-S-aminoacyloxymethyl mercaptopurine derivatives, 6-mercaptopurine (6-MP), 7,8-polymethyleneimidazo-1,3,2-diazaph-osphorines, azathioprine, methyl-D-glucopyranoside mercaptopurine derivatives and s-alkynyl mercaptopurine derivatives; indoline ring and a modified ornithine or glutamic acid-bearing methotrexate derivatives, alkyl-substituted benzene ring C bearing methotrexate derivatives, benzoxazine or benzothiazine moiety-bearing methotrexate derivatives, 10-deazaminopterin analogues, 5-deazaminopterin and 5,10-dideazaminopterin methotrexate analogues, indoline moiety-bearing methotrexate derivatives, lipophilic amide methotrexate derivatives, L-threo-(2S,4S)-4-fluoro-glutamic acid and DL-3,3-difluoroglutamic acid-containing methotrexate analogues, methotrexate tetrahydroquinazoline analogue, N-(ac-aminoacyl) methotrexate derivatives, biotin methotrexate derivatives, D-glutamic acid or D-erythrou, threo-4-fluoroglutamic acid methotrexate analogues, β,γ-methano methotrexate analogues, 10-deazaminopterin (10-EDAM) analogue, γ-tetrazole methotrexate analogue, N-(L-α-aminoacyl) methotrexate derivatives, meta and ortho isomers of aminopterin, hydroxymethylmethotrexate (DE 267495), γ-fluoromethotrexate, polyglutamyl methotrexate derivatives, gem-diphosphonate methotrexate analogues (WO 88/06158), α- and γ-substituted methotrexate analogues, 5-methyl-5-deaza methotrexate analogues (4,725,687), N.delta.-acyl-N α-(4-amino-4-deoxypteroyl)-L-ornithine derivatives, 8-deaza methotrexate analogues, acivicin methotrexate analogue, polymeric platinol methotrexate derivative, methotrexate-γ-dimyristoylphophatidylethanolamine, methotrexate polyglutamate analogues, poly-γ-glutamyl methotrexate derivatives, deoxyuridylate methotrexate derivatives, iodoacetyl lysine methotrexate analogue, 2, .omega.-diaminoalkanoid acid-containing methotrexate analogues, polyglutamate methotrexate derivatives, 5-methyl-5-deaza analogues, quinazoline methotrexate analogue, pyrazine methotrexate analogue, cysteic acid and homocysteic acid methotrexate analogues (4,490,529), γ-tert-butyl methotrexate esters, fluorinated methotrexate analogues, folate methotrexate analogue, phosphonoglutamic acid analogues, poly (L-lysine) methotrexate conjugates, dilysine and trilysine methotrexate derivates, 7-hydroxymethotrexate, poly-γ-glutamyl methotrexate analogues, 3′,5′-dichloromethotrexate, diazoketone and chloromethylketone methotrexate analogues, 10-propargylaminopterin and alkyl methotrexate homologs, lectin derivatives of methotrexate, polyglutamate methotrexate derivatives, halogentated methotrexate derivatives, 8-alkyl-7,8-dihydro analogues, 7-methyl methotrexate derivatives and dichloromethotrexate, lipophilic methotrexate derivatives and 3′,5′-dichloromethotrexate, deaza amethopterin analogues, MX068 and cysteic acid and homocysteic acid methotrexate analogues (EPA 0142220); N3-alkylated analogues of 5-fluorouracil, 5-fluorouracil derivatives with 1,4-oxaheteroepane moieties, 5-fluorouracil and nucleoside analogues, cis- and trans-5-fluoro-5,6-dihydro-6-alkoxyuracil, cyclopentane 5-fluorouracil analogues, A-OT-fluorouracil, N4-trimethoxybenzoyl-5′-deoxy-5-fluoro-cytidine and 5′-deoxy-5-fluorouridine, 1-hexylcarbamoyl-5-fluorouracil, B-3839, uracil-1-(2-tetrahydrofuryl)-5-fluorouracil, 1-(2′-deoxy-2′-fluoro-β-D-arabinofuranosyl)-5-fl-uorouracil, doxifluridine, 5′-deoxy-5-fluorouridine, 1-acetyl-3-O-toluoyl-5-fluorouracil, 5-fluorouracil-m-formylbenzene-sulfonate (JP 55059173), N-(2-furanidyl)-5-fluorouracil (JP 53149985) and 1-(2-tetrahydrofuryl)-5-fluorouracil (JP 52089680); 4′-epidoxorubicin; N-substituted deacetylvinblastine amide (vindesine) sulfates; and Cu(II)-VP-16 (etoposide) complex, pyrrolecarboxamidino-bearing etoposide analogues, 40-amino etoposide analogues, γ-lactone ring-modified arylamino etoposide analogues, N-glucosyl etoposide analogue, etoposide A-ring analogues, 4′-deshydroxy-4′-methyl etoposide, pendulum ring etoposide analogues and E-ring desoxy etoposide analogues.

In one embodiment, the cytotoxic agent is a small drug molecule (Payne et al., U.S. Pat. No. 7,202,346). In another embodiment, the cytotoxic agent a maytansinoid, an analog of a maytansinoid, a prodrug of a maytansinoid, or a prodrug of an analog of a maytansinoid (U.S. Pat. Nos. 6,333,410; 5,475,092; 5,585,499; 5,846,545; 7,202,346). In another embodiment, the cytotoxic agent may be a taxane (see U.S. Pat. Nos. 6,340,701 & 6,372,738 & 7,202,346) or CC-1065 analog (see U.S. Pat. Nos. 5,846,545; 5,585,499; 5,475,092 & 7,202,346).

In another embodiment, the cytotoxic agent is exemplified by an auristatin, a DNA minor groove binding agent, a DNA minor groove alkylating agent, an enediyne, a duocarmycin, a maytansinoid, and a vinca alkaloid (U.S. Pat. No. 7,662,387).

In a further embodiment, the cytotoxic agent is an anti-tubulin agent (U.S. Pat. No. 7,662,387). In yet another embodiment, the cytotoxic agent is exemplified by dimethylvaline-valine-dolaisoleuine-dolaproine-phenylalanine-p-phenylenediamine (AFP), dovaline-valine-dolaisoleunine-dolaproine-phenylalanine (MMAF), and monomethyl auristatin E (MAE) (U.S. Pat. No. 7,662,387).

In an additional embodiment the cytotoxic agent is exemplified by radioisotope emitting radiation, immunomodulator, lectin, and toxin (U.S. Pat. No. 6,429,295). In particular, the radioisotope emitting radiation is an alpha-emitter selected from the group consisting of ²¹²Bi, ²¹³Bi, and ²¹¹M, or a beta-emitter selected from the group consisting of ¹⁸⁶Re and ⁹⁰Y, or a gamma-emitter ¹³¹I (U.S. Pat. No. 7,666,425).

In one embodiment, the toxin is exemplified by ricin, the A-chain of ricin, and pokeweed antiviral protein (U.S. Pat. No. 5,057,313).

Anti-cancer toxins are further exemplified by methotrexate, 5-fluorouracil, cycloheximide, daunomycin, doxorubicin, chlorambucil, trenimon, phenylenediamine mustard, adriamycin, bleomycin, cytosine arabinoside or Cyclophosphamide (U.S. Pat. No. 5,057,13). Further representative examples of anti-cancer toxins include taxanes (e.g., paclitaxel and docetaxel). Etanidazole, Nimorazole, perfluorochemicals with hyperbaric oxygen, transfusion, erythropoietin, BW12C, nicotinamide, hydralazine, BSO, WR-2721, IudR, DUdR, etanidazole, WR-2721, BSO, mono-substituted keto-aldehyde compounds, nitroimidazole, 5-substituted-4-nitroimidazoles, SR-2508, 2H-isoindolediones (U.S. Pat. No. 4,494,547), chiral (((2-bromoethyl)-amino)methyl)-nitro-1H-imidazole-1-ethanol (U.S. Pat. No. 5,543,527; U.S. Pat. No. 4,797,397; U.S. Pat. No. 5,342,959), nitroaniline derivatives (U.S. Pat. No. 5,571,845), DNA-affinic hypoxia selective cytotoxins (U.S. Pat. No. 5,602,142), halogenated DNA ligand (U.S. Pat. No. 5,641,764), 1,2,4 benzotriazine oxides (U.S. Pat. No. 5,616,584; U.S. Pat. No. 5,624,925; U.S. Pat. No. 5,175,287), nitric oxide (U.S. Pat. No. 5,650,442), 2-nitroimidazole derivatives (U.S. Pat. No. 4,797,397; U.S. Pat. No. 5,270,330; U.S. Pat. No. 5,270,330; Patent EP 0 513 351 B1), fluorine-containing nitroazole derivatives (U.S. Pat. No. 4,927,941), copper (U.S. Pat. No. 5,100,885), combination modality cancer therapy (U.S. Pat. No. 4,681,091), 5-CldC or (d)H.sub.4U an/or 5-halo-2′-halo-2′-deoxy-cytidine and/or -uridine derivatives (U.S. Pat. No. 4,894,364), platinum complexes (U.S. Pat. No. 4,921,963; Patent EP 0 287 317 A3), fluorine-containing nitroazole (U.S. Pat. No. 4,927,941), benzamide, autobiotics (U.S. Pat. No. 5,147,652), benzamide and nicotinamide (U.S. Pat. No. 5,215,738), acridine-intercalator (U.S. Pat. No. 5,294,715), fluorine-containing nitroimidazole (U.S. Pat. No. 5,304,654, Apr. 19, 1994), hydroxylated texaphyrins (U.S. Pat. No. 5,457,183), hydroxylated compound derivative (Publication Number 011106775 A (Japan), Oct. 22, 1987; Publication Number 01139596 A (Japan), Nov. 25, 1987; Publication Number 63170375 A (Japan)), fluorine containing 3-nitro-1,2,4-triazole (Publication Number 02076861 A (Japan), Mar. 31, 1988), 5-thiotretrazole derivative or its salt (Publication Number 61010511 A (Japan), Jun. 26, 1984), Nitrothiazole (Publication Number 61167616 A (Japan) Jan. 22, 1985), imidazole derivatives (Publication Number 6203767 A (Japan) Aug. 1, 1985; Publication Number 62030768 A (Japan) Aug. 1, 1985; Publication Number 62030777 A (Japan) Aug. 1, 1985), 4-nitro-1,2,3-triazole (Publication Number 62039525 A (Japan), Aug. 15, 1985), 3-nitro-1,2,4-triazole (Publication Number 62138427 A (Japan), Dec. 12, 1985), Carcinostatic action regulator (Publication Number 63099017 A (Japan), Nov. 21, 1986), 4,5-dinitroimidazole derivative (Publication Number 63310873 A (Japan) Jun. 9, 1987), nitrotriazole Compound (Publication Number 07149737 A (Japan) Jun. 22, 1993), cisplatin, doxorubin, misonidazole, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide (Tannock. Journal of Clinical Oncology 14(12):3156-3174, 1996), camptothecin (Ewend et al. Cancer Research 56(22):5217-5223, 1996) and paclitaxel (Tishler et al. Journal of Radiation Oncology and Biological Physics 22(3):613-617, 1992).

In a preferred embodiment, the anti-cancer toxin comprises folate, e.g., for applications in breast cancer treatment. Data herein demonstrate that adenovirus metabolically labeled with azido sugar O-GlcNAz that is linked to folate, showed a 3 to 4 fold increase in transgene delivery to murine breast cancer cells.

iii) Therapeutic Molecule

In another embodiment, the molecule of interest comprises a therapeutic molecule. The team “therapeutic molecule” refers to a molecule that reduces, delays and/or eliminates undesirable pathologic effects in a cell, tissue, organ and/or animal.

Therapeutic molecules are exemplified by therapeutic sequences (e.g., therapeutic nucleotide sequences and/or the encoded therapeutic polypeptides), which may be homologous or heterologous with respect to the sequences of the target cell into which they are introduced.

Homologous therapeutic sequences are useful for expressing wild-type proteins where it is desirable to, for example, compensate for either insufficient expression of a wild-type protein product in the cell or to bring about expression of a mutant protein product whose biological activity is reduced relative to the wild-type protein.

Heterologous therapeutic sequences are useful in, for example, expressing a mutant protein which is less active, more active, and/or more stable, than the wild-type protein. Alternatively, heterologous therapeutic nucleotide sequences may be used to express a heterologous protein which is derived from a species that is different from the target cell species, such that the expressed heterologous protein complements or supplies a deficient activity in the target cell, thus allowing the latter to resist a pathological process, or else stimulate an immune response.

Another use of heterologous therapeutic nucleotide sequences is in the generation of vaccines against microorganisms (e.g., viruses, bacteria, etc.) or against cancer cells. This may be achieved, for example, where the nucleotide sequence of interest encodes an antigenic peptide which is capable of generating an immune response in a host animal or human, or which encodes variable regions from specific antibodies and immunomodulator genes. For example, the encoded antigenic polypeptides may be derived from the Epstein Barr virus, the HIV virus, the hepatitis B virus (such as those described in patent EP 185 573), or the pseudorabies virus. Alternatively, the antigenic polypeptides may be specific for tumors (such as those described in patent EP 259 212).

Illustrative therapeutic sequences include, but are not limited to, polypeptide sequences and/or nucleotide sequences encoding them, such as enzymes; lymphokines (e.g., interleukins, interferons, TNF, etc.); growth factors (e.g., erythropoietin, G-CSF, M-CSF, GM-CSF, etc.); neurotransmitters or their precursors or enzymes responsible for synthesizing them; trophic factors (e.g., BDNF, CNTF, NGF, IGF, GMF, aFGF, bFGF, NT3, NT5, HARP/pleiotrophin, etc.); apolipoproteins (e.g., ApoAI, ApoAIV, ApoE. etc.); lipoprotein lipase (LPL); the tumor-suppressing genes (e.g., p53, Rb, Rap1A, DCC k-rev, etc.); factors involved in blood coagulation (e.g., Factor VII, Factor VIII, Factor IX, etc.); DNA repair enzymes; suicide genes (thymidine kinase or cytosine deaminase); blood products; hormones; etc.

In one preferred embodiment, the therapeutic nucleotide sequence encodes a wild-type gene for which a mutant has been associated with a human disease. Such wild-type genes may be used for “gene replacement therapy,” i.e., replacement of defective genes. Therapeutic nucleotide sequence encoding a wild-type gene are exemplified, but not limited to, the adenosine deaminase (ADA) gene (GenBank Accession No. M13792 associated with adenosine deaminase deficiency with severe combined immune deficiency; alpha-1-antitrypsin gene (GenBank Accession No. M11465) associated with alpha1-antitrypsin deficiency; beta chain of hemoglobin gene (GenBank Accession No. NM_{—}000518) associated with beta thalassemia and Sickle cell disease; receptor for low density lipoprotein gene (GenBank Accession No. D16494) associated with familial hypercholesterolemia; lysosomal glucocerebrosidase gene (GenBank Accession No. K02920) associated with Gaucher disease; hypoxanthine-guanine phosphoribosyltransferase (HPRT) gene (GenBank Accession No. M26434, J00205, M27558, M27559, M27560, M27561, M29753, M29754, M29755, M29756, M29757) associated with Lesch-Nyhan syndrome; lysosomal arylsulfatase A (ARSA) gene (GenBank Accession No. NM_{—}000487) associated with metachromatic leukodystrophy; ornithine transcarbamylase (OTC) gene (GenBank Accession No. NM_{—}000531) associated with ornithine transcarbamylase deficiency; phenylalanine hydroxylase (PAH) gene (GenBank Accession No. NM_{—}000277) associated with phenylketonuria; purine nucleoside phosphorylase (NP) gene (GenBank Accession No. NM_{—}000270) associated with purine nucleoside phosphorylase deficiency; the dystrophin gene (GenBank Accession Nos. M18533, M17154, and M18026) associated with muscular dystrophy; the utrophin (also called the dystrophin related protein) gene (GenBank Accession No. NM_{—}007124) whose protein product has been reported to be capable of functionally substituting for the dystrophin gene; and the human cystic fibrosis transmembrane conductance regulator (CFTR) gene (GenBank Accession No. M28668) associated with cystic fibrosis. In a preferred embodiment, the therapeutic gene is human Factor VIII.

In one embodiment, the therapeutic nucleotide sequence is a “suicide gene,” i.e., a gene encoding “suicide protein” such as an enzyme that can metabolize a separately administered non-toxic pro-drug into a potent cytotoxin, which can diffuse to and kill neighboring cells. A herpes simplex virus, encoding a thymidine kinase suicide gene, has progressed to phase III clinical trials. The herpes simplex virus thymidine kinase phosphorylates the pro-drug, gancyclovir, which is then incorporated into DNA, blocking DNA synthesis. In one preferred embodiment, the virus is an oncovirus for preferential delivery and expression of the suicide genes in cancer cells.

In another embodiment, the therapeutic nucleotide sequence encodes an “anti-angiogenesis protein,” i.e., a protein (e.g., angiostatin and endostatin) that reduces angiogenesis, thereby resulting in oxygen starvation of the cancer. The infection of cells with viruses containing the genes for angiostatin and endostatin synthesis inhibited cancer growth in mice.

iv) Antibody and Antigen-Binding Fragments

In a further embodiment, the molecule of interest comprises an antibody and/or an antigen-binding fragment of an antibody. Viruses thus modified are useful in, for example, detection of disease in diagnostic applications, as well as treatment of disease in therapeutic applications.

The term “antibody” refers to an immunoglobulin (e.g., IgG, IgM, IgA, IgE, IgD, etc.). The basic functional unit of each antibody is an immunoglobulin (Ig) monomer (containing only one immunoglobulin (“Ig”) unit). Included within this definition are monoclonal antibodies, chimeric antibodies, recombinant antibodies, and humanized antibodies.

In one embodiment, the invention's antibodies are monoclonal antibodies produced by hybridoma cells.

In particular, the invention contemplates antibody fragments that contain the idiotype (“antigen-binding fragment”) of the antibody molecule. For example, such fragments include, but are not limited to, the Fab region, F(ab′)2 fragment, pFc′ fragment, and Fab′ fragments.

The “Fab region” and “fragment, antigen binding region,” interchangeably refer to portion of the antibody arms of the immunoglobulin “Y” that function in binding antigen. The Fab region is composed of one constant and one variable domain from each heavy and light chain of the antibody. Methods are known in the art for the construction of Fab expression libraries (Huse et al., Science, 246:1275-1281 (1989)) to allow rapid and easy identification of monoclonal Fab fragments with the desired specificity. In another embodiment, Fc and Fab fragments can be generated by using the enzyme papain to cleave an immunoglobulin monomer into two Fab fragments and an Fc fragment. The enzyme pepsin cleaves below the hinge region, so a “F(ab′)2 fragment” and a “pFc′ fragment” is formed. The F(ab′)2 fragment can be split into two “Fab′ fragments” by mild reduction.

The invention also contemplates a “single-chain antibody” fragment, i.e., an amino acid sequence having at least one of the variable or complementarity determining regions (CDRs) of the whole antibody, and lacking some or all of the constant domains of the antibody. These constant domains are not necessary for antigen binding, but constitute a major portion of the structure of whole antibodies. Single-chain antibody fragments are smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely to provoke an immune response in a recipient than whole antibodies. Techniques for the production of single-chain antibodies are known (U.S. Pat. No. 4,946,778). The variable regions of the heavy and light chains can be fused together to form a “single-chain variable fragment” (“scFv fragment”), which is only half the size of the Fab fragment, yet retains the original specificity of the parent immunoglobulin.

The “Fc” and “Fragment, crystallizable” region interchangeably refer to portion of the base of the immunoglobulin “Y” that function in role in modulating immune cell activity. The Fc region is composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. By doing this, it mediates different physiological effects including opsonization, cell lysis, and degranulation of mast cells, basophils and eosinophils. In an experimental setting, Fc and Fab fragments can be generated in the laboratory by cleaving an immunoglobulin monomer with the enzyme papain into two Fab fragments and an Fc fragment.

v) Affibody

In another embodiment, the molecule of interest comprises an affibody. Viruses thus modified find use in, for example, detection of disease in diagnostic applications, as well as treatment of disease in therapeutic applications.

“Affinity body,” “Affibody®,” and “affibody” molecules are antibody mimetic proteins that, like antibodies, can specifically bind target antigens (Nord, K., et al. (1997) Nature Biotechnol. 15: 772-777). Affibody molecules can be designed and used like aptamers. In one embodiment, Affibody molecules comprise a backbone derived from an IgG-binding domain of Staphylococcal Protein A (Protein A produced by S. aureus). The backbone can be derived from an IgG binding domain comprising the three alpha helices of the IgG-binding domain of Staphlococcal Protein A termed the B domain. The amino acid sequence of the B domain is described in Uhlen et al., J. Biol. Chem. 259: 1695-1702 (1984). Alternatively, the backbone can be derived from the three alpha helices of the synthetic IgG-binding domain known in the art as the Z domain, which is described in Nilsson et al., Protein Eng. 1: 107-113 (1987). The backbone of an affibody comprises the amino acid sequences of the IgG binding domain with amino acid substitutions at one or more amino acid positions. The affibody, for example, comprises the 58 amino acid sequence of the Z domain (VDNKFNKEXXXAXXEIXXLPNLNXXQXXAHXSLXDDPSQSANLLAEAKKLNDAQAPK), wherein X at each of positions 9, 10, 11, 13, 14, 17, 18, 24, 25, 27, 28, 32, and 35 is any amino acid (Capala et al., U.S. Pat. Appl. No. US20100254899).

The affibody molecule constitutes a highly suitable carrier for directing molecules of interest (e.g., toxins, radioisotopes, therapeutic peptides) to tumor cells due to specific target binding and lack of irrelevant interactions, such as the Fc receptor binding displayed by some antibodies.

Common advantages of Affibody® molecules over antibodies are better solubility, tissue penetration, stability towards heat and enzymes, and comparatively low production costs.

Affibodies are exemplified by, but not limited to, Anti-ErbB2 Affibody® (also referred to as anti-HER2 Affibody®), Anti-EGFR Affibody®, Anti-TNF alpha Affibody®, Anti-fibrinogen Affibody®, Anti-transferrin Affibody®, Anti-HSA Affibody®, Anti-Insulin Affibody®, Anti-IgG Affibody®, Anti-IgM Affibody®, Anti-IgA Affibody®, and Anti-IgE Affibody® (e.g., from Abcam, Cambridge, Mass.).

Affibodies with an affinity of down to sub-nanomolar have been obtained from naïve library selections, and affibodies with picomolar affinity have been obtained following affinity maturation (Orlova et al. (2006). “Tumor imaging using a picomolar affinity HER2 binding affibody molecule”. Cancer Res. 66 (8): 4339-48. PMID 16618759). Affibodies conjugated to weak electrophiles bind their targets covalently (Holm et al., Electrophilic affibodies forming covalent bonds to protein targets, J Biol Chem. 2009 Nov. 20; 284(47):32906-13. PMID 19759009).

Affibody molecules can be synthesized chemically or in bacteria or purchased from a commercial source (e.g., Affibody AB, Bromma, Sweden; Abcam, Cambridge, Mass.).

Affibody molecules can also be obtained by constructing a library of affibodies as described in U.S. Pat. No. 5,831,012, which is incorporated herein by reference. The affibody library can then be screened for affibodies which bind to target antigens of interest (e.g., HER-2, EGFR) by methods known in the art.

Affibody molecules are based on a three-helix bundle domain, which can be expressed in soluble and proteolytically stable forms in various host cells on its own or via fusion with other protein partners (Ståhl et al. (1997). “The use of gene fusions to protein A and protein G in immunology and biotechnology”. Pathol. Biol. (Paris) 45: 66-76. PMID 9097850.”

Affibodies tolerate modification and are independently folding when incorporated into fusion proteins. Head-to-tail fusions of Affibody molecules of the same specificity have proven to give avidity effects in target binding, and head-to-tail fusion of Affibody molecules of different specificities makes it possible to get bi-specific or multi-specific affinity proteins. Fusions with other proteins can also be created (Rönnmark et al. (2002) “Construction and characterization of affibody-Fc chimeras produced in Escherichia coli,” J. Immunol. Methods 261: 199-211. PMID 11861078; Rönnmark et al. (2003) “Affibody-beta-galactosidase immunoconjugates produced as soluble fusion proteins in the Escherichia coli cytosol,” J. Immunol. Methods 281: 149-160. PMID 14580889). A site for site-specific conjugation is facilitated by introduction of a single cysteine at a desired position.

A number of different Affibody molecules have been produced by chemical synthesis. Since they do not contain cysteines or disulfide bridges, they fold spontaneously and reversibly into the correct three-dimensional structures when the protection groups are removed after synthesis (Nord et al. (2001) “Recombinant human factor VIII-specific affinity ligands selected from phage-displayed combinatorial libraries of protein A,” Eur. J. Biochem. 268: 1-10. PMID 11488921; Engfeldt et al. (2005) “Chemical synthesis of triple-labeled three-helix bundle binding proteins for specific fluorescent detection of unlabeled protein,” Chem. BioChem. 6: 1043-1050. PMID 15880677).

In a particularly preferred embodiment, the affibody comprises anti ErbB-2 affibody as exemplified by anti ErbB-2 affibody conjugated to Adenovirus (AdLUC) (Example 9).

vi) Antigens and Epitopes

In further embodiments, the molecule of interest comprises an antigen and/or epitope. Viruses thus modified are useful in, for example, detection of disease in diagnostic applications, as well as treatment of disease in vaccine therapy applications.

The terms “antigen,” “immunogen,” “antigenic,” “immunogenic,” “antigenically active,” “immunologic,” and “immunologically active” when made in reference to a molecule, refer to any substance that is capable of inducing a specific humoral immune response (including eliciting a soluble antibody response) and/or cell-mediated immune response (including eliciting a CTL response). In one embodiment the antigen is exemplified by Human Immunodeficiency virus gag protein, malaria circumsporozite protein (CSP_{full}) antigen, malaria CSP T cell epitope (EYLNKIQNSLSTEWSPCSVT; U.S. Pat. No. 6,669,945), malaria CSP B Cell epitope (NANPNANPNANPNANPNANPNANP; WO 2009/082440 A2), and Pseudomonas antigen.

In some embodiments, the antigen polypeptide is “pathogen derived,” meaning expressed by a pathogen (e.g., bacteria, virus, parasite, protozoan, fungus, etc.), such as Herpes virus, Neisseria gonorrhea, Treponema, Escherichia coli, Respiratory Syncytial virus, tuberculosis, Streptococcus, Chlamydia, and Ebola virus. Pathogen derived antigens are exemplified by Human Immunodeficiency virus (HIV) gag protein (including the HXB2 strain gag protein (Genbank Accession #K03455), HIV Gag protein antigen such as HIV Gap protein immunodominant peptide AMQMLKETI (WO 2010/051820 A1), HIV Pol protein antigen, HIV Nef protein antigen, malaria circumsporozite protein (CSP_{full}) antigen, malaria CSP T cell epitope (EYLNKIQNSLSTEWSPCSVT; U.S. Pat. No. 6,669,945), malaria CSP B Cell epitope (NANPNANPNANPNANPNANPNANP; WO 2009/082440 A2), and Pseudomonas antigen.

The terms “epitope” and “antigenic determinant” refer to a structure on an antigen, which interacts with the binding site of an antibody or T cell receptor as a result of molecular complementarity. An epitope may compete with the intact antigen, from which it is derived, for binding to an antibody. Generally, secreted antibodies and their corresponding membrane-bound forms are capable of recognizing a wide variety of substances as antigens, whereas T cell receptors are capable of recognizing only fragments of proteins which are complexed with MHC molecules on cell surfaces. Antigens recognized by immunoglobulin receptors on B cells are subdivided into three categories: T-cell dependent antigens, type 1 T cell-independent antigens; and type 2 T cell-independent antigens. Also, for example, when a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

Exemplary epitopes include, without limitation YPYDVPDYA (U.S. Pat. No. 7,255,859), EphrinA2 epitopes from renal cell carcinoma and prostate cancer (U.S. Pat. No. 7,297,337), hepatitis C virus epitopes (U.S. Pat. Nos. 7,238,356 and 7,220,420), vaccinia virus epitopes (U.S. Pat. No. 7,217,526), dog dander epitopes (U.S. Pat. No. 7,166,291), human papilloma virus (HPV) epitopes (U.S. Pat. Nos. 7,153,659 and 6,900,035), Mycobacterium tuberculosis epitopes (U.S. Pat. Nos. 7,037,510 and 6,991,797), bacterial meningitis epitopes (U.S. Pat. No. 7,018,637), malaria epitopes (U.S. Pat. No. 6,942,866), and type 1 diabetes mellitus epitopes (U.S. Pat. No. 6,930,181).

B) Vaccines

The invention provides compositions (such as vaccines) comprising one or more of the invention's modified viruses (e.g., viruses having an external surface covalently linked to at least one heterologous unnatural moiety) (and/or hybrid viruses and/or recombinant viruses). In one embodiment, the composition is free of helper virus. The term “vaccine” refers to a pharmaceutically acceptable preparation that may be administered to a host to induce a humoral immune response (including eliciting a soluble antibody response) and/or cell-mediated immune response (including eliciting a cytotoxic T lymphocyte (CTL) response).

In one embodiment, the composition further comprises a pharmaceutically acceptable compound such as diluent, carrier, excipient, and/or adjuvant.

C) Kits

The invention further provides a kit comprising a virus and at least one heterologous unnatural moiety selected from unnatural amino acid and unnatural saccharide. In particular embodiments, the kit further comprises instructions on modifying the virus with the unnatural moiety to produce a modified (e.g., infectious) virus having an external surface covalently linked to the unnatural moiety.

Some viruses may be more easily produced by transfecting producer cells with plasmids encoding the viral genes. Thus, the kit optionally may include the unnatural molecules of interest, reagents necessary for the reaction and the plasmids without the actual viruses.

D) Exemplary Methods for Generating the Invention's Compositions

The invention provides a method for producing a modified (e.g., infectious) virus having an external surface covalently linked to an unnatural moiety, comprising contacting i) a virus, ii) host cell susceptible to the virus, and iii) at least one unnatural moiety selected from unnatural amino acid and unnatural saccharide, wherein the contacting is under conditions for infection of the host cell by the virus to produce a treated cell that comprises a modified (e.g., infectious) virus having an external surface covalently linked to the unnatural moiety. Thus, in one embodiment, the invention provides a method for producing a modified infectious virus having an external surface covalently linked to an unnatural moiety, comprising i) contacting a virus with a host cell susceptible to said virus, wherein said contacting is under conditions for infection of said host cell by said virus to produce an infected cell that comprises said virus, and ii) contacting said infected cell with at least one unnatural moiety selected from unnatural amino acid and unnatural saccharide, wherein said contacting is under conditions for covalently linking an external surface of said virus, that is comprised in said infected cell, with said unnatural moiety. This is exemplified below in Example 1, “metabolic labeling of adenovirus type 5 with azidohomoalanine,” and in Example 8 “Metabolic labeling of Adenovirus type 5 with N-azidoacetygalactosamine.”

While not intending to limit the type or source of host cell, in one embodiment the susceptible host cell is permissive to the virus, and the modified (e.g., infectious) virus is released from the treated cell.

While not necessary to the invention's methods, in one embodiment it may be desirable to detect the unnatural moiety on the external surface of the modified (e.g., infectious) virus (see Example 11).

While not necessary to the invention's methods, in some embodiments it may be desirable to determine the level of viability of the treated cell. Preferably, the treated cell has substantially the same viability as a control host cell infected with the virus in the absence of the unnatural moiety. Data herein demonstrate that producer cell viability was unaltered when infected with adenovirus that is modified with the unnatural monosaccharide azido analog O-GlcNAz compared to cell (Example 12) or with the unnatural amino acid, azidohomoalanine (AHA) (Examples 1-7).

While not necessary to the invention's methods, the methods optionally may further comprise determining the level of infection of the treated cells by the modified (e.g., infectious) virus (e.g., as shown in Example 12). Preferably, the modified infectious virus has substantially the same level of infectivity as a control virus that lacks the unnatural moiety. Data herein demonstrated that AHA incorporation in adenovirus has no adverse impact on virus production and infectivity (Examples 1-7), and that incorporation of O-GlcNAz in adenovirus also had no adverse effect on virus production and infectivity (Example 12).

While not necessary to the invention's methods, the invention's methods may optionally further comprise determining the level of replication of the modified (e.g., infectious) virus in the treated cells. In preferred embodiments, the level of replication of the modified infectious virus in the treated cell is substantially the same as the level of replication of a control virus that lacks the unnatural moiety. Data herein demonstrated that AHA incorporation in adenovirus has no adverse impact on virus production and infectivity (Examples 1-7) and that incorporation of O-GlcNAz in adenovirus also had no adverse effect on virus production and infectivity (Example 12).

In some embodiments, the level of replication of the modified (e.g., infectious) virus in the treated cell is greater than the level of replication of a control virus that lacks the unnatural moiety. For example, level of replication of the modified infectious virus in the treated cell is from 1.5 fold to 1,000 fold greater than the level of replication of the control virus that lacks the unnatural moiety. Data herein demonstrate that breast cancer cells infected with adenovirus that is metabolically modified with surface incorporation of O-GlcNAz linked to folate showed a 3-4 fold increased replication by the modified virus compared to replication following infection with control unmodified virus (Example 14).

In a further embodiment, the invention's methods may further comprise determining the level of productive infection of the treated cell by the modified (e.g., infectious) virus. In particular embodiments, the modified (e.g., infectious) virus has substantially the same level of productive infectivity as a control virus that lacks the unnatural moiety. Data herein demonstrated that AHA incorporation in adenovirus has no adverse impact on virus production and infectivity (Examples 1-7) and also that incorporation of O-GlcNAz in adenovirus had no adverse effect on virus production and infectivity (Example 12).

In some embodiments it may be desirable to purify the modified (e.g., infectious) virus produced by the invention's methods (e.g., Example 10).

The invention contemplates modified (e.g., infectious) viruses produced by any of the invention's methods disclosed herein.

E) Exemplary Diagnostic Applications for the Invention's Compositions

The invention provides a method for detecting disease in a subject comprising contacting tissue of the subject with any one or more of the invention's compositions (e.g., modified virus having an external surface covalently linked to at least one heterologous unnatural moiety), wherein a) the unnatural moiety is covalently linked to a molecule of interest, b) contacting is under conditions for specific binding of the molecule of interest with a second molecule in the tissue, and c) the subject is identified as having disease when an altered level of the specific binding is detected relative to a control normal tissue, and the subject is identified as being disease-free when the level of the specific binding is unaltered relative to a control normal tissue. In some embodiments, the molecule of interest is exemplified by antibody, affibody, and antigen, and optional may further contain a probe.

In some embodiments it may be desirable, though not necessary, to determine the level of specific binding of the molecule of interest with one or more of the tissue compared to a control normal tissue.

F) Exemplary Therapeutic Applications for the Invention's Compositions

The invention provides a method for reducing one or more symptoms of disease in a subject, comprising administering to the subject a therapeutic amount of any one or more of the invention's compositions (e.g., modified virus having an external surface covalently linked to at least one heterologous unnatural moiety) to produce a treated subject, wherein the unnatural moiety is covalently linked to a molecule of interest. In some embodiments, the molecule of interest is exemplified by cytotoxin, therapeutic molecule, antibody, affibody, and antigen.

The invention's compositions (such as viruses, having an external surface covalently linked to at least one heterologous unnatural moiety) are administered in a therapeutic amount. The terms “therapeutic amount,” “pharmaceutically effective amount,” “therapeutically effective amount,” “biologically effective amount,” and “protective amount” are used interchangeably herein to refer to an amount that is sufficient to achieve a desired result, whether quantitative and/or qualitative. In particular, a therapeutic amount is that amount that delays, reduces, palliates, ameliorates, stabilizes, prevents and/or reverses one or more symptoms of the disease compared to in the absence of the composition of interest. Examples include, without limitation, tumor size and/or tumor number in cancer disease, glucose levels in blood and/or urine in diabetes, standard biochemical kidney function tests in kidney disease, etc. The terms also include, in another embodiment, an amount of the composition that reduces infection by a pathogen (e.g., HIV, malaria parasite, Pseudomonas species), regardless of whether disease symptoms are altered (i.e., increased or reduced).

In vaccine applications, the invention's compositions are preferably administered in an immunologically effective amount. In one embodiment, “immunogenically effective amount” and “immunologically-effective amount” refer to that amount of a molecule that elicits and/or increases production of an immune response (including production of specific antibodies and/or induction of a cytotoxic T lymphocyte (CTL) response) in a host upon vaccination.

Specific “dosages” can be readily determined by clinical trials and depend, for example, on the route of administration, patient weight (e.g. milligrams of drug per kg body weight). The term “delaying” symptoms refers to increasing the time period between exposure to the immunogen or virus and the onset of one or more symptoms of the exposure. The term “eliminating” symptoms refers to 100% reduction of one or more symptoms of exposure to the immunogen or virus.

As used herein, the actual amount, i.e., “dosage,” encompassed by the term “pharmaceutically effective amount,” “therapeutically effective amount,” “immunologically effective,” and “protective amount” will depend on the route of administration, the type of subject being treated, and the physical characteristics of the specific subject under consideration. These factors and their relationship to determining this amount are well known to skilled practitioners in the medical, veterinary, and other related arts. This amount and the method of administration can be tailored to achieve optimal efficacy but will depend on such factors as weight, diet, concurrent medication and other factors that those skilled in the art will recognize. The dosage amount and frequency are selected to create an effective level of the compound without substantially harmful effects.

Administration of the invention's compositions may be by intramuscular administration, intradermal administration, intravenous administration, subcutaneous administration, aerosol administration, oral administration, and/or sub-lingual administration,

The compositions of the invention may be administered before, concomitantly with, and/or after manifestation of one or more symptoms of a disease or condition, and/or after administration of another type of drug or therapeutic procedure (e.g., surgery, radiation, etc.). Administration may be in vivo and/or or ex vivo.

Optionally, the method may further comprise detecting a reduction in one or more symptoms of the disease in the treated subject.

The invention's therapeutic methods include gene therapy applications, e.g., where the invention's viruses are administered under conditions for integration (such as site-specific integration) of the virus into the genome of one or more cells in the treated subject. These applications may employ therapeutic molecules, such as a nucleotide sequence encoding a wild type amino acid sequence, a nucleotide sequence encoding a suicide protein, and/or a nucleotide sequence encoding an anti-angiogenesis protein.

The invention's therapeutic methods include oncolytic therapy applications, such as in cancer therapy.

The invention's therapeutic methods include vaccine therapy applications, for example where the molecule of interest comprises a heterologous antigenic sequence that is administered to a subject in an immunologically effective amount. Exemplary heterologous antigenic sequences include amino acid sequences and/or nucleotide sequence encoding an amino acid sequence.

### EXPERIMENTAL

The following examples serve to illustrate certain embodiments and aspects of the present invention and are not to be construed as limiting the scope thereof.

### Example 1

### Materials and Methods Used in Examples 2-7

All chemical reagents were obtained from commercial sources and used without further purification unless otherwise noted. NMR spectra were recorded on a Varian 300 MHz NMR spectrometer or Varian 400 MHz NMR spectrometer. Mass spectra for the small molecules were obtained using an Agilent 1100 LC/MSD VL instrument. Thin Layer Chromatography (TLC) was performed on Merck DC-alufolien with Kieselgel 60E-254 and column chromatography was carried out on silica gel 60 (Merck; 230-400 mesh ASTM). RP-HPLC was performed using a L201243 Shimadzu on a C12 Jupiter column (250×10 mm; Phenomenex). UV-Visible absorbance was recorded on a Beckmann Coulter DU 730. Electrophoresis gels were scanned on a Typhoon 9400 fluorescent gel scanner.

Synthesis of Azidohomoalanine (Aha).

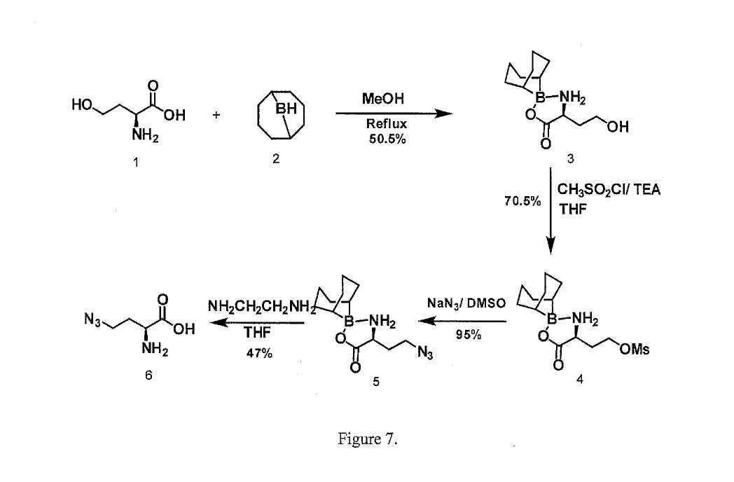

Azidohomoalanine was synthesized in four steps as described (FIG. 7):

Compound 3.

L-Homoserine (1.45 g, 12.7 mmol) was added to a solution of 9-Borabicyclo(3.3.1)nonane or 9-BBN (1.51 g, 12.4 mmol) in methanol (25 mL). The reaction mixture was refluxed for 3 hours under argon, at which time TLC analysis showed the reaction to be complete. The reaction mixture was concentrated and the crude product was purified by silica gel chromatography using a gradient elution (50% ethyl acetate-hexane to 100% ethyl acetate). 9-BBN protected L-homoserine was obtained as a white solid in 51% yield and purity was assessed as a single spot in TLC and ¹H NMR. ¹H NMR (300 MHz, CD₃OD): δ 1.38-1.50 (2H, m), 1.60-1.98 (13H, m), 2.25 (1H, m), 3.75-3.85 (3H, m) ppm. ESI-MS calculated for C₁₂H₂₂BO₃N (MH⁺) 239.81 found at 239.1.

Compound 4.

Methylsulfonyl chloride (0.87 mL, 7.5 mmol) was added to a solution of compound 3 (1.5 g, 6.3 mmol) in tetrahydrofurane (20 mL) followed by triethylamine (1.2 mL, 12 mmol) at 0° C. The reaction mixture was warmed to room temperature and stirred overnight. Subsequent TLC analysis showed the reaction to be complete. The resulting mixture was concentrated and purified by silica gel chromatography with gradient elution (50% ethyl acetate-hexane to 100% ethyl acetate). The product was obtained as a white solid in 70% yield. Purity was ascertained by TLC and ¹H NMR. ¹H NMR (300 MHz, CD₃OD): δ 1.38-1.50 (2H, m), 1.60-1.98 (12H, m), 2.15 (1H, m), 2.45 (1H, m), 3.12 (3H, s), 3.86 (1H, t), 4.40-4.58 (2H, m) ppm. ESI-MS calculated for C₁₃H₂₄BSO₄ (MH⁺) 319.81, found 319.1.

Compound 5.

Sodium azide (0.5 g, 7 mmol) was added to a solution of compound 4 (1.5 g, 5 mmol) in dimethylsulfoxide (10 mL). The mixture was stirred at 60° C. for 3 hours. The mixture formed a thick solid that was extracted into ethyl acetate and washed with water. The product was light yellow oil obtained in 95% yield. ¹H NMR (300 MHz, CD₃OD): δ 1.38-1.50 (2H, m), 1.60-1.98 (13H, m), 2.20 (1H, m), 3.55-3.70 (2H, m), 3.75 (1H, t) ppm. ESI-MS calculated for C₁₂H₂₂BO₂N₄ (MH⁺) 264.1, found 264.1.

Azidohomoalanine (compound 6).

Ethylene diamine (1 mL, 21 mmol) was added to a solution of compound 5 (1.5 g, 5.6 mmol) in tetrahydrofurane (10 mL) and refluxed for 2 hours. The mixture was concentrated and triturated with ethyl acetate. This was filtered through a glass column over sand and cotton. The solid residue was dissolved in methanol and purified by silica gel chromatography eluting with 50:30:20 ethyl acetate: methanol: water. The pure amino acid was obtained as white solid in 47% yield. ¹H and 2D NMR confirmed its purity. ¹H NMR (300Mz, D₂O): δ 1.92-2.12 (2H, m), 3.42-3.50 (2H, m), 3.72 (1H, t) ppm. ESI-MS calculated for C₄H₈O₂N₄ (MH⁺) 145.1, found 145.1.

Synthesis of alk-PEG-folate.

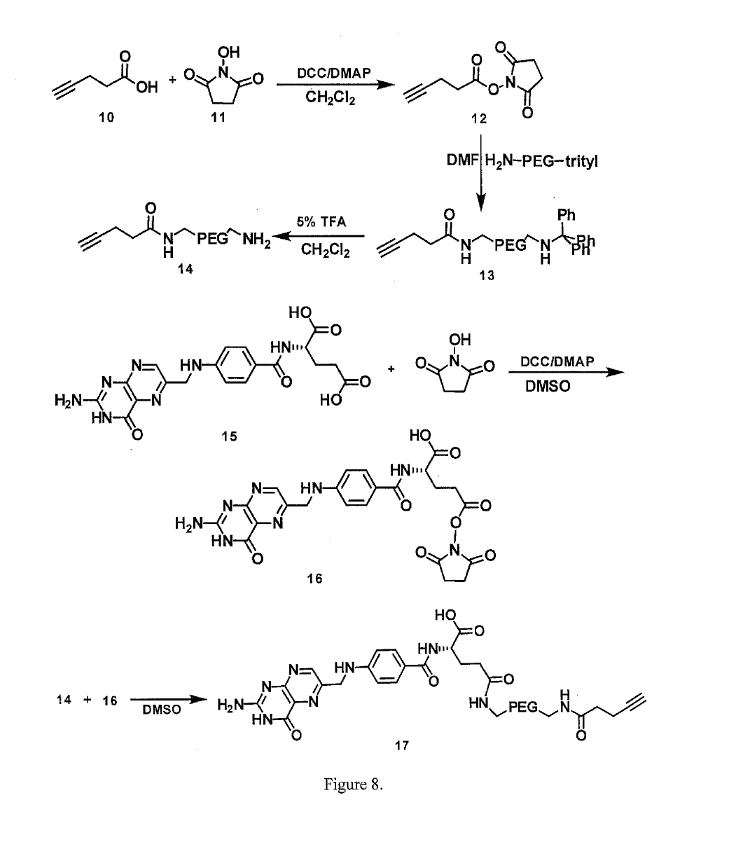

(FIG. 8) Pentynoic acid (200 mg, 2.0 mmol) was activated by N,N′-dicyclohexylcarbodiimide (824 mg, 4.0 mmol) and N-hydroxysuccinimide (460 mg, 4.0 mmol) in methylene chloride at RT for 12 hours and filtered with a syringe filter (pore size 0.2 μm). O—(N-trityl-3-aminopropyl)-O′-(3-aminpropyl)-diethyleneglycol (MW 462) was added to the solution at a molar ratio of 5:1 (alkyne:PEG) and stirred vigorously overnight. The resulting solution was acidified with trifluoroaceticacid (final concentration 1%) to remove C-protecting trityl group. The reaction was then neutralized with triethyl amine and concentrated in vacuo. Folate NHS ester was added to the alkyne-PEG under an atmosphere of argon in DMSO and allowed to react for 12 hours. The alkyne-PEG-folate was precipitated by addition of diethyl ether. Trituration with DMSO/ether was repeated 3 times. Crude product was purified on a Jupiter C-12 column with a gradient of 5% to 25% acetonitrile containing 0.1% TFA. Product eluted at 30 minutes. ¹H NMR (300 Mz, D₂O): δ 1.07 (2H, t), 1.10 (4H, t), 1.89 (4H, m), 1.92 (12H, m), 2.71 (1H, s), 2.87 (1H, s), 3.40 (2H, m), 3.50-3.51 (2H, m), 3.97 (4H, q), 6.7 (2H, d), 7.5 (2H, d), 9.0 (1H, s) ppm. ESIMS calculated for C₃₄H₄₅O₉N₉ (MH⁺) 724.3, found 724.3.

Synthesis of Alkyne and Phosphine FLAG.

Synthesized as described by Bertozzi et. al. (14). Briefly, all peptides were synthesized by standard Fmoc solid phase peptide synthesis protocol. In this case activated esters were formed using N,N′-diisopropylcarbodiimide and 1-Hydroxybenzotriazole. Alkyne functionalized peptides were produced by on-bead N-terminal functionalization with propynoic acid. Phosphine FLAG was obtained by on-bead N-terminal derivitization with 2-(Diphenylphosphino)terephthalic acid 1-methyl 4-pentafluorophenyl diester (Sigma-Aldrich 679011). During all de-blocking steps 0.1 M HOBt was added to the 20% piperidine solution to alleviate aspartamide formation.

Cell Culture and Plaque Assays.

Dulbecco's modified Eagle's medium (DMEM), RPMI 1640, penicillin/streptomycin and 0.5% trypsin-EDTA were purchased from GIBCO (Grand Island, N.Y.). RPMI 1640 minus folic acid was obtained from Sigma. Fetal calf serum (FCS) and bovine calf serum (BCS) was purchased from HyClone (Logan, Utah). 293 cells were maintained in DMEM supplemented with 10% BCS, 2 mM glutamine, 100 U/mL penicillin, 100 μg/mL streptomycin. 4T1 cells (ATCC) were maintained in RPMI 1640 medium containing 10% FCS, 2 mM L-glutamine, 100 U/mL penicillin, and 100 μg/mL streptomycin. For 4T1 cells grown without folate, RPMI 1640 minus folic acid was supplemented with 10% FCS, 0.3 mg/mL L-glutamine, 2 mg/mL sodium bicarbonate, 100 U/mL penicillin, 100 μg/mL streptomycin. All cells were maintained in 100×20 mm tissue culture dishes obtained from BD biosciences (Franklin Lakes, N.J.) at 37° C. and 5% CO₂. HEK 293 cells in 60 mL culture plates were infected with 400 μL of azide labeled virus particles at concentrations of 10³ particles/mL. The plates were overlaid with complete media containing 2.8% bactoagar and 10% BCS. After 3 days the cells were again overlaid with the same agar solution containing 2% BCS. After the 6^{th} day when plaques became visible the cells were overlaid with agar solution containing 0.1% neutral red. The plaques were counted within a day after the final overlay.

Metabolic Labeling of Adenovirus Type 5 with Azidohomoalanine.

HEK 293 cells were infected with wild type adenovirus particles with an MOI of 5 pfu/cell. 18 hours post infection complete media was removed and the cells washed with TD buffer (25 mM Tris, 125 mM NaCl, 5 mM KCl and 1 mM Na₂HPO₄ at pH 7.5) at 37° C. for 20 minutes. Aha supplemented DMEM (−Met) media was then added to the infected cells and allowed to grow until 24 hours post infection. The labeling media was then removed and the cells were supplemented with complete media. The plates were harvested 46-48 hours post infection and virus particles purified over a gradient of 1.4 g/mL and 1.25 g/mL CsCl centrifuged using an SW41 rotor (Beckman) at 32,000 rpm for 1 hour at 15° C. The virus band at the junction of the two CsCl bands was collected and further purified using an SW60 rotor (Beckman) by an 18 hour centrifugation at 35,000 rpm over 1.33 g/mL CsCl.

Reaction of Azide Labeled Virus with Alk-TAMRA.

A solution of 50 μL of 1×10¹² azide labeled virus particles/mL in 100 mM tris pH 8.0, with bathophenanthroline disulphonic acid disodium salt (3 mM) and alk-TAMRA (400 μM), 5-tetramethylrhodamine (GBiosciences, Maryland Heights, Mo.) was kept in a argon-filled glove bag for 6 hours to deoxygenate. After which copper bromide at a final concentration of 1 mM was added to the mixture and the reaction allowed to proceed for 12 hours (7) inside the glove bag. The samples were then removed from the glove bag and quenched by addition of 10 mM EDTA. All samples were then analyzed by fluorescent gel scanning to determine the extent of chemical labeling.

Reaction of Azide Labeled Virus with Phosphine-FLAG and Western Blotting.

Virus particles containing metabolically incorporated azidohomoalanine were treated with phosphine-FLAG at a final concentration of 500 μM for 3 hours. Aha enabled virus samples coupled with FLAG were run on a 10% SDS polyacrylamide electrophoresis gel and transferred onto nitrocellulose at 40V over 2 hours in a western transfer buffer (25 mM Tris, 192 mM glycine, 0.5% SDS and 10% methanol). Blots were blocked by 5% milk in PBST and treated with anti-FLAG M2 HRP conjugate (Sigma Chemical) at a ratio of 1:12000 in 5% milk in PBST. Blots were washed with milk and PBST and developed by chemiluminescence (Millipore Immobilon Western kit). For IR fluorescence the blots were treated with an anti-penton antibody followed by an IR 680 dye as secondary and the blots visualized on the Odyssey LICOR, excitation at 680 nm and emission at 700±15 nm.

MS analysis. Matrix-assisted laser desorption/ionization (MALDI) mass spectra were obtained using a Bruker Autoflex II MALDI/TOF/TOF. LC-MS sequencing of the viral peptides were performed on a Thermo Fisher Scientific LTQ XL. Virus particles labeled metabolically with Aha were treated with 8 M urea to dissociate the particles and denature viral proteins. The solution was diluted with ammonium bicarbonate to 0.5 M urea. The resulting solution was treated with 0.1 μg/μL of trypsin at 1:50 (protein:protease) ratio and digestion was allowed to proceed overnight at 37° C. The reaction was quenched with 1/10^{th} volume of 100% formic acid and concentrated to 1/10^{th} the reaction volume. The peptide mixture was subjected to LC/MS treatment using a C18 column. MS data was searched against the SPROT database with Inspect for the following modifications C+57 (iodoacetamide adduct of cysteine), M+16 (methionine oxidation), M-5 (methionine substituted by azidohomoalanine. Molecular weight of Aha being 144.1, it is 5 mass units less than methionine) and STY+80 (serine, threonine and tyrosine phosphorylation). For quantification of Aha incorporation, elution profiles of daughter ions that should be the same in both the modified and unmodified peptides were traced and areas calculated.

Fluorescent Gel Scanning Assay.

Azide enabled viral particles (with 4 mM and 32 mM Aha) were labeled with an alk-TAMRA dye using Cu catalyzed “click” chemistry, as described above for FLAG labeling. The particles were purified using Centri-Sep spin columns and quantified with QuantIT Picogreen Dye labeling (17). 1×10⁹ particles were run on an electrophoresis gel using alk-TAMRA as a quantization standard. A series of alk-TAMRA dilutions were loaded 10 minutes before the end of the run. Gels were scanned using a typhoon gel scanner in the fluorescence mode with excitation filter at 532 nm and emission filter at 580±15 nm. The scans were subsequently analyzed with Image Quant TL 1D gel analyzer software. All gels were run in the dark at 4° C. and scanned within 10 minutes of the end of run.

Targeting Assay and Fluorescent Microscopy.

Ad5, encoding GFP or luciferase in the E1 region, were labeled with 4 mM and 32 mM Aha as described. Viruses were labeled with folate bearing alkyne probe (alk-PEG-folate) using Cu catalyzed “click” chemistry in a deoxygenated glove under same conditions (as for TAMRA labeling) and the reaction quenched with 10 mM EDTA. Viruses were purified on G-25 sephedex desalting spin columns (Centri Sep spin columns; Adelphia, N.J.), quantified using QuantIT Picogreen assay (Molecular Probes, Eugene, Oreg.), and stored in a 0.9 mM CaCl₂ and 0.5 mM MgCl₂ buffer in PBS containing 10% glycerol. Mouse breast cancer cell line 4T1 was cultivated in minus folate media for 2 weeks after which they were seeded in 24 well plates at a density of 1×10⁶ cells/well. 24 hours after replating the cells were infected with metabolically and chemically labeled virus at an MOI of 50. 24 hours post infection; Luciferase expression was evaluated using a Perkin Elmer chemiluminesence plate reader (excitation 485±10 nm; emission 528±10 nm). 4T1 cells cultivated in minus folate media for 2 weeks were seeded in glass bottom dishes at 1.1×10⁵ cells per dish. Cells were infected with folate labeled Ad5 or the negative control, no metabolic Aha incorporation, both bearing the GFP transgene as described for the targeting assay. Notably, all viruses were exposed to the chemical labeling conditions irrespective of the presence of Aha. 24 hours post infection the dishes were mounted and cells imaged by confocal microscopy in a Zeiss LSM 510 equipped with a 63×, 1.4 NA C-Apochromat water immersion objective. Images were processed using Zeiss LSM 510 software.

### Example 2

### Production and Characterization of Aha Labeled Adenovirus Particles

Metabolic incorporation of Aha was accomplished by production of adenovirus particles in the presence of methionine-free medium containing the free, unnatural amino acid. Specifically, the inventors infected HEK 293 cells with adenovirus type 5 particles at an MOI of 5. Eighteen hours post infection, growth media was removed from the cells and the cells washed with Tris buffer. Methionine-free media, supplemented with 4 mM Aha (−Met/+Aha), was added to each plate of infected cells and the infection allowed to proceed for six hours. At this time the −Met/+Aha media was removed and substituted with complete media until the cells were harvested for virus. At 48 hours post-infection the cells were harvested, lysed and the virus was purified by CsCl equilibrium gradient centrifugation. In order to generate the appropriate controls, particle production was also carried out with 4 mM methionine and a mixture of 4:1 Aha:Met labeling media.

Incorporation of the non-natural amino acid in the Ad5 capsid was assessed by mass spectrometry analysis. Virus particles were ethanol precipitated, subjected to urea denaturation, following which they were trypsin digested and analyzed on a LC/MS system. MS analysis revealed a number of predicted peptides demonstrated a −5 m/z shift, which is consistent with Met replacement by Aha. In all cases, these peptides were found to co-elute with peptides that corresponded to the natural Met containing sequence. Mixtures of Aha/Met containing peptides indicate that Aha replacement of Met is incomplete, which was expected given the inventors' partial metabolic labeling conditions. All of the putative Aha containing peptides were subjected to MS/MS analysis. In all cases, the −5 m/z shift was found to occur at positions corresponding to Met codons confirming the incorporation of Aha. It was tempting to quantitate Aha incorporation via direct comparison of Aha and Met containing peptides. However, this analysis demonstrated very different results indicating either that the incorporation is influenced by sequence, folding constraints or that ionization is impacted by Aha incorporation in a non-linear manner. The latter conclusion is supported by chemical labeling, which largely reflects the product of protein copy number and methionine occupancy. As a result, the inventors have no direct evidence indicating bias in Aha incorporation.

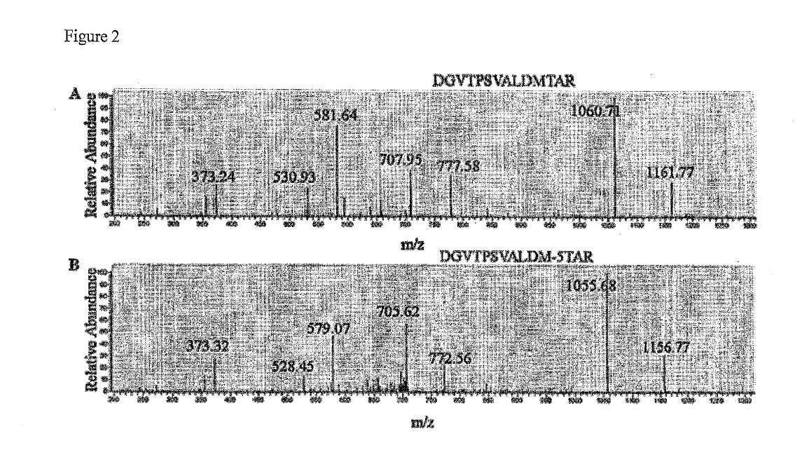

A number of viral peptides were observed to incorporate the M-5 modification (Aha being 5 mass units less than Met) as shown in FIG. 2, details of peptides incorporating Aha is listed in Table 1. Sequencing of the flagged M-5 peptides revealed the mass change occurred at methionine codons. Furthermore, analogous peptide determination of the amount of incorporation was challenging with the LC/MS approach due to inherent differences in peptide migration under mass spectrometry conditions. Thus the inventors decided to focus on the percent of chemically modifiable residues, which would present a more appropriate picture of the attachment sites available due to Aha incorporation.

TABLE 1
List of the peptides identified after Inspect search following LC/MS
treatment of trypsin digestion of Aha labeled adenovirus particles

| | | parent | parent | labeled | labeled |
|---|---|---|---|---|---|
| peptide sequence | charge | peak | mass | parent mass | parent peak |
| R.WSLDYMDNVNPFNHHR.N | 3 | 682.31 | 2044.92 | 2039.92 | 680.64 |
| R.AAAAAAAAISAMTQGR.R | 2 | 716.37 | 1431.74 | 1426.74 | 713.87 |
| R.DGVTPSVALDMTAR.N | 2 | 716.86 | 1432.72 | 1427.72 | 714.36 |
| R.YVQQSVSLNLMR.D | 2 | 719.38 | 1437.76 | 1432.76 | 716.88 |
| R.SMLLGNGRYVPFHIQVPQK.F | 3 | 728.73 | 2184.19 | 2179.19 | 727.06 |
| R.LSEPLVTSNGMLALK.M | 2 | 786.93 | 1572.86 | 1567.86 | 784.43 |
| K.ISDNPNTYDYMNK.R | 2 | 787.84 | 1574.69 | 1569.69 | 785.34 |
| R.ESGDLAPTVQLMVPK.R | 2 | 792.92 | 1584.84 | 1579.84 | 790.42 |
| R.MYSFFRNFQPMSR.Q | 2 | 855.90 | 1710.80 | 1705.80 | 853.40 |
| K.IGHGLEFDSNKAMVPK.L | 2 | 871.95 | 1742.90 | 1737.90 | 869.45 |
| R.AQQQGNLGSMVALNAFLSTQPANV | 3 | 904.80 | 2712.40 | 2707.40 | 903.13 |
| PR.G | | | | | |
| K.VVLYSEDVDIETPDTHISYMPTIK.E | 3 | 922.46 | 2765.37 | 2760.37 | 920.79 |
| R.LMVTETPQSEVYQSGPDYFFQTSR. | 3 | 937.44 | 2810.31 | 2805.31 | 935.77 |
| Q | | | | | |
| K.GLMFDATAIAINAGDGLEFGSPNAP | 3 | 1115.90 | 3345.69 | 3340.69 | 1114.23 |
| NTNPLKTK.I | | | | | |

### Example 3

### Chemical Modification of Aha Labeled Virus

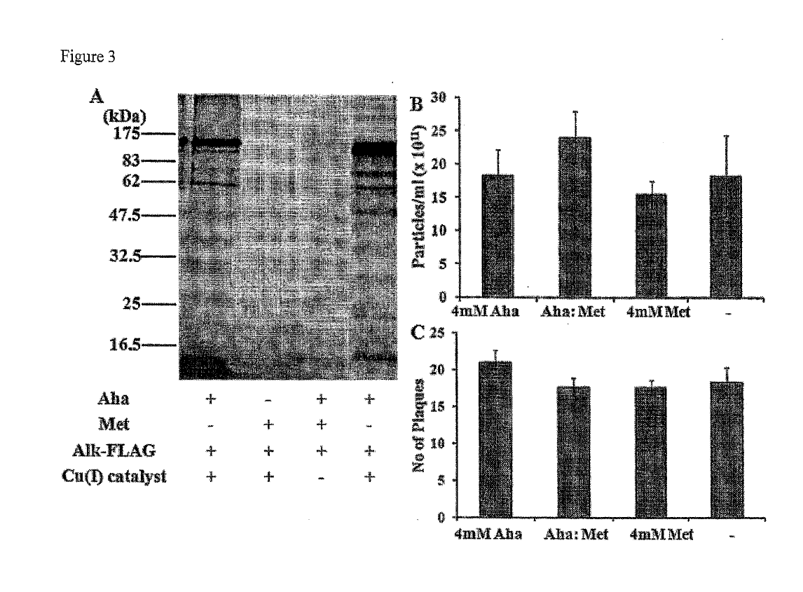

For specific chemical labeling of azides, three different reaction techniques have been developed. Copper assisted “click” reaction, the Staudinger ligation reaction and the strain promoted electrocyclization. For the inventors' experiments the inventors have used both the copper assisted “click” reaction and the Staudinger ligation reaction. For this the purified Ad5 viral particles were subjected to copper assisted azide alkyne cycloaddition reaction (7) with an alk-TAMRA ligand. Reaction was carried out in a deoxygenated glove bag overnight in the presence of 1 mM copper (I) bromide and 3 mM SBP (bathophenanthroline disulphonic acid disodium salt) ligand. Fluorescent gel scanning was performed on the whole virus particle run on a SDS-PAGE gel. Gel scanning showed strong labeling on a number of the adenoviral capsid proteins (FIG. 3A) for samples labeled with 4 mM Aha. No signal was observed with metabolically unlabeled virus or “click” reactions carried out in the absence of copper. Virus particles denatured before chemical labeling showed greater dye labeling on a number of viral proteins with couple of the core proteins also showing chemical modification. TAMRA labeling suggests modification occurs in hexon, penton, fiber and pIIIa on the intact virus. Together these data suggest that adenoviral capsid proteins have specific incorporation at the methionine sites with azidohomoalanine which result in their being chemically modified at solvent exposed surfaces.

### Example 4

### Assessment of Viral Fitness

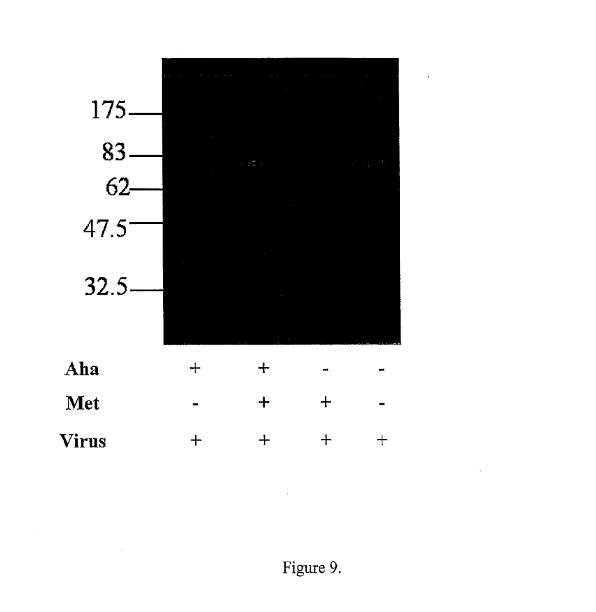

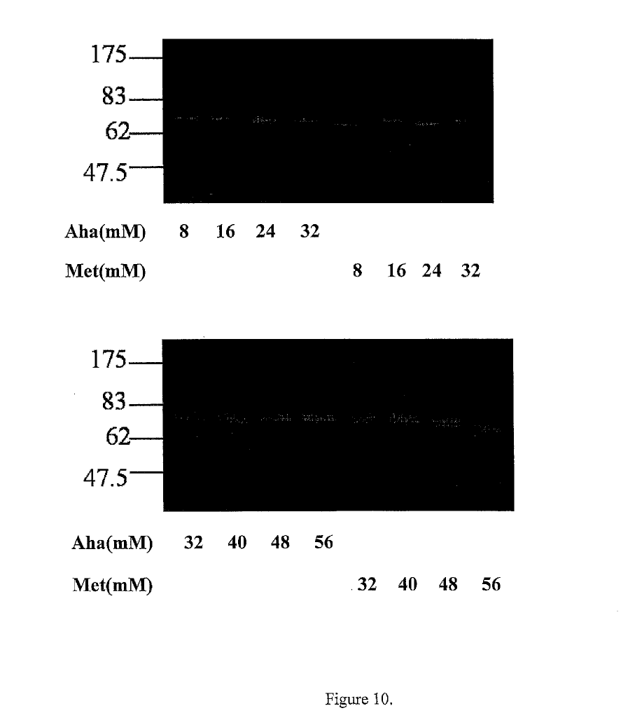

In order to discern the impact of Aha incorporation on viral fitness, virus particle production and standard plaque forming assays were performed. There was no discernable effect of Aha incorporation in the viral particle production as estimated by standard UV absorption assay (FIG. 3B). In addition, plaque forming ability of metabolically labeled virion was equivalent to unlabeled virus (FIG. 3C). To estimate if viral protein expression was somehow hampered during the period of Aha exposure, Ad5 infected cells labeled for 6 hours (18 to 24 hours post infection) with 4 mM Aha were lysed 24 hours after infection and analyzed for penton production by western blot with an anti-penton antibody. Development of the blot with an IR fluorescent dye showed no loss in penton expression during labeled infection as compared to particles produced in complete media or in methionine-free media with exogenous methionine supplied (FIGS. 9 and 10). Taken together, normal protein expression through the metabolic labeling period, standard particle production and fully infective viruses indicate that Aha has little impact on either structural protein folding or particle virus assembly. While this is surprising at a superficial level, a closer examination of the surface charge and steric occupancy of Met and Aha demonstrate that they are very similar (10).

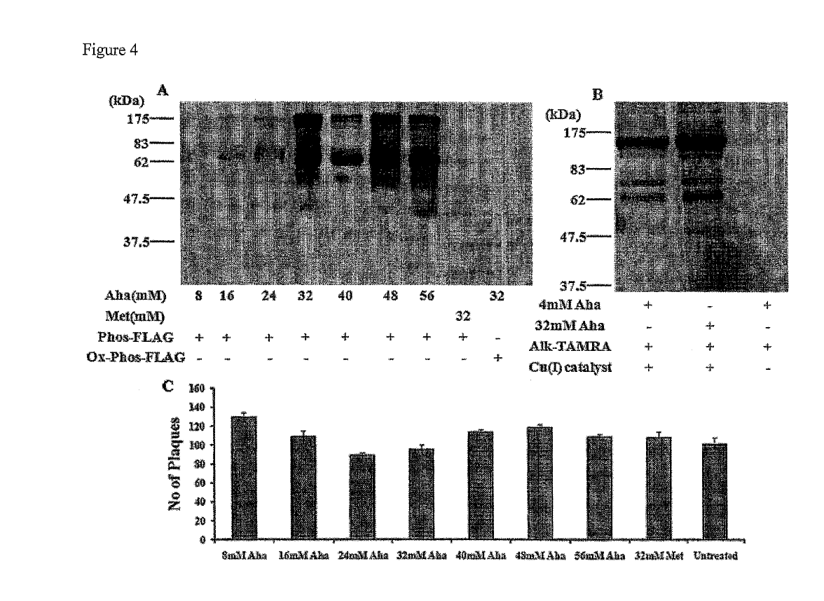

As virus production seemed unaffected while labeling with 4 mM Aha, the inventors wanted to assess whether an increase in the concentration of the non-natural amino acid in the growth media reflects an increase in azide dependent labeling and whether this results in any detrimental effects on virus growth and infectivity. Thus the inventors repeated the labeled infection process with 8 mM, 16 mM, 24 mM, 32 mM, 48 mM and 52 mM Aha supplemented methionine-free media from 18 to 24 hours post-infection. Staundinger ligation reaction with FLAG peptide bearing phosphine probe of labeled cell lysate 24 hours after infection and subsequent anti-FLAG western analysis with anti-FLAG antibody demonstrated saturation of metabolic labeling at 32 mM (FIG. 4A). Plaque assays were also performed in parallel with increasing Aha labeled virus. The results show that there occurs no effect on the infectivity of these azide enabled particles even at 56 mM Aha. This suggests that Aha is not toxic to the cells and does not hamper normal cellular processes even at high concentration. Also virus particles labeled with high levels of Aha seem to behave normally with respect to particle stability and infectivity (FIG. 4B). For subsequent quantification of Aha incorporation and targeting of these tagged viruses towards cancerous cell types, both 4 mM as well as 32 mM Aha labeled virus samples were used.

### Example 5

### Quantification of Chemical Labeling of Aha-Enabled hAd5

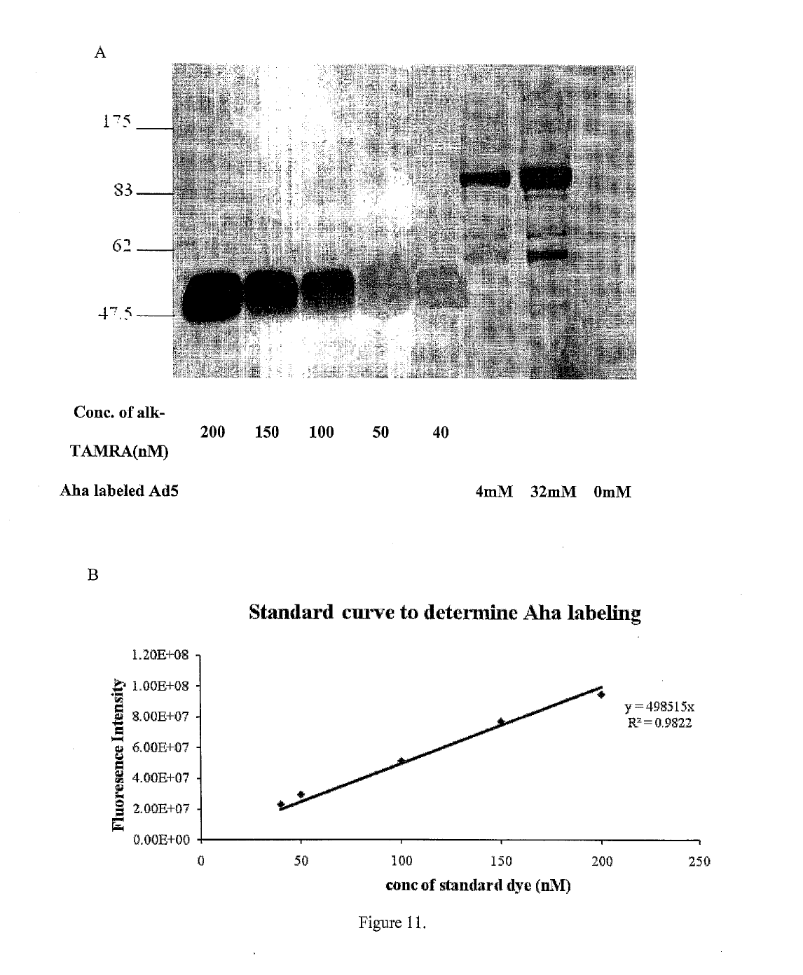

The exposed surface of adenovirus capsid is thought to be composed of five structural proteins based on X-ray crystallographic and cryo-EM structural data (4, 23, 24). Of these, hexon, the most abundant capsid protein is about one-third solvent exposed surface area (22, 26). The penton and fiber proteins also are largely solvent accessible (18, 28). In contrast, protein IIIa and IX have only small exposed segments (5, 16, 25). As chemical derivatization was performed on intact particles, modification of AHA by alkyne probes is expected to occur only when solvent exposed. In addition, the number of attached probes on the viral capsid should be proportional to the effective percent incorporation of azidohomoalanine within the viral proteins. The number of exposed methionine sites within the three main structural proteins, hexon, penton and fiber are 8, 5 and 3 respectively, estimated by analysis of X-ray structures for these proteins. As shown in Table 2 this analysis estimates ˜6000 exposed methionine on the adenoviral capsid. To calculate the number of modifiable azides on the adenovirus capsid, the 4 mM and 32 mM Aha-labeled viral particles were modified with a fluorophore, an alkyne-tetramethyl rhodamine dye (alk-TAMRA) using copper assisted “click” chemistry. The modified virus was separated from excess catalyst and probe on a spin column and the subsequent particle count estimated with QuantIT picogreen assay (17). TAMRA labeled particles were run on 10% SDS PAGE and analyzed on a fluorescent gel scanner. Known concentrations of alk-TAMRA were also run as standard (FIG. 4C, FIG. 11). The scanned gel was analyzed with Image Quant TL1D gel analyzer software and the average number of dye molecules on 3 major coat proteins with three independent reactions was calculated (Table 2). The average total number of modified sites on each 4 mM and 32 mM Aha labeled virus being 279.5±8.6 and 512.0±7.8 respectively. Thus covering about 5.5% and 10% of the estimated total exposed methionine sites for adenovirus type5 (Table 2).

TABLE 2

| | Total no. of | Estimated sol- | | Estimated | Average number of | |
|---|---|---|---|---|---|---|
| Adenoviral | methionine | vent exposed | Copies of | total no of | chemically labeled | |
| structural | residues per | methionine | protein | solvent exposed | dyes per VP | |
| proteins* | polypeptide | (from crystal str.) | per VP | MET per VP | 4 mM Aha | 32 mM Aha |
| Hexon | 20 | 8 | 720 | 5760 | 196.1 ± 4.6 | 358.3 ± 10.4 |
| Penton | 12 | 5 | 60 | 300 | 36.6 ± 1.8 | 57.3 ± 3.6 |
| Fiber | 10 | 3 | 36 | 108 | 47.1 ± 2.8 | 96.5 ± 0.7 |

*only these 3 protein X-ray crystal structures were used to estimate the average number of exposed methionine residues per VP

### Example 6

### Folate Mediated Infection of Mouse Breast Cancer Cell Lines

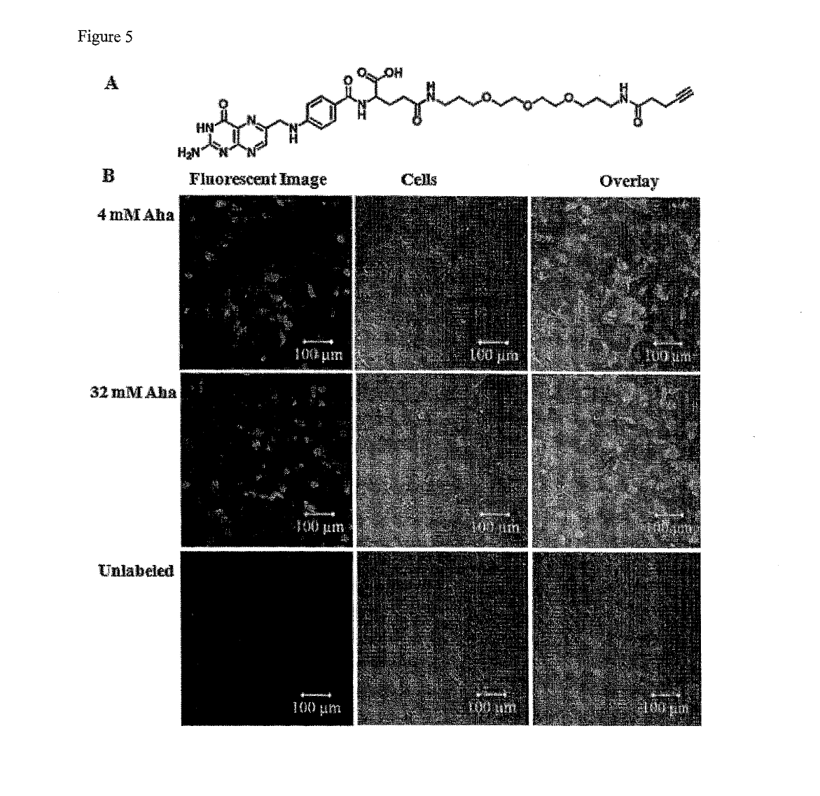

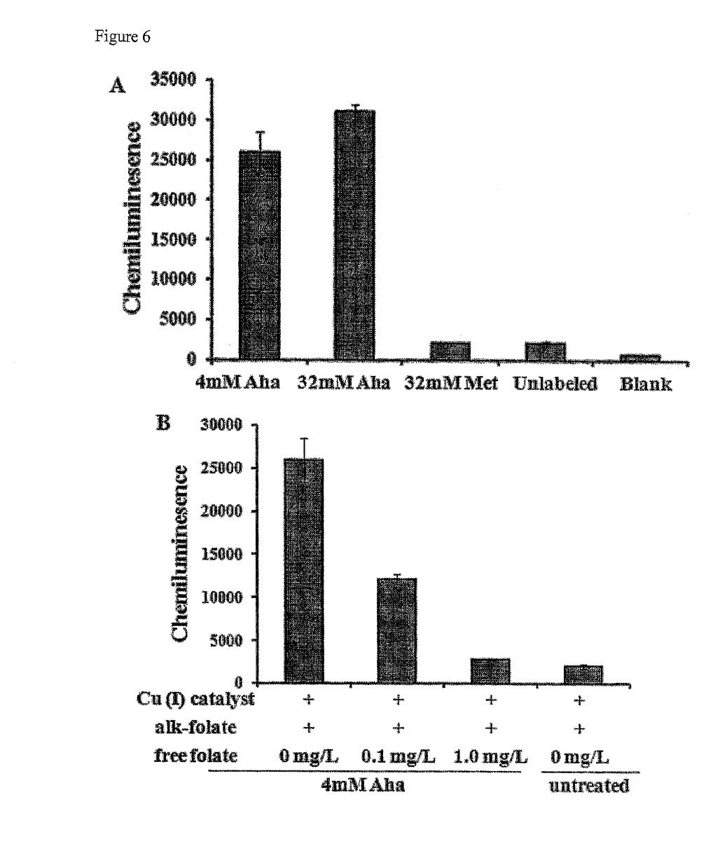

To evaluate the potential of using sites of azide introduction for targeting element attachment, the inventors modified Aha-enabled hAd5 with alk-folate. Folate conjugates have been extensively used over the past decade to target cytotoxic and imaging agents to a number of different types of cancer (13). Advantageously, the folate receptor (FR) has high affinity for folate conjugates, mediates endocytosis, is restricted to the apical surface of polarized epithelium (except in the kidneys), and is over expressed in variety of tumors (21). Breast carcinoma cells 4T1, of murine origin, reported to have over expressed folate receptors were used for the inventors' work. Use of folic acid in retargeting of adenoviruses has also been attempted by the attachment of PEGylated folate to lysine residues on adenoviral coat proteins (19). The inventors followed a similar targeting approach by designing a folate-alkyne ligand with a 462 MW PEG as linker (FIG. 5A, FIG. 8). A GFP or Luciferase transgene bearing Ad5 was produced in the presence of 4 mM and 32 mM of Aha as has been described earlier, metabolically unlabeled and methionine labeled particles were used as control. The targeting molecule was “clicked” onto azide enabled adenoviral particles with GFP or Luciferase as reporter and the virus purified on a Centrisep spin column and quantified with QuantIT picogreen. Confocal microscopy of 4T1 cells infected with folate labeled and metabolically unlabeled virus was also performed using a Zeiss LSM 510. For this 1.1×10⁵ 4T1 cells were seeded in glass bottom dishes and infected with the modified and unmodified virus particles. 24 hours post-infection the cells were imaged and data analyzed in a Zeiss LSM 510 software. The results (FIG. 5B) show a high level of GFP expression in these breast cancerous cell types with the folate targeted virus as compared to the unmodified particles. For quantification of transgene expression, 4T1 cells were grown on minus folate media for 2 weeks after which time they were seeded in 24 well plates at a concentration of 1×10⁶ cells per well. They were infected with labeled adenovirus the next day at an MOI of 50 and Luciferase expression was used to monitor transductional retargeting ability 24 hours post-infection using a Perkin Elmer Chemiluminesence plate reader (FIG. 6). The data showed a tenfold increase in transgene expression with the modified adenoviral particles compared to the unmodified samples. Competition experiments with free folic acid showed loss of Luciferase activity at higher folate concentrations. Overall the targeting results with the fluorescent microscopy and luciferase quantification experiments suggest effective retargeting of the viral particles towards folate receptor over expressing murine breast cancer cell types.

### Example 7

### Discussion of Results from Examples 1-6

Despite being heavily developed for gene therapy, the use of adenovirus vectors has been hampered by the inability to easily tune virus-host interactions. Here, the inventors report a strategy that introduces unnatural amino acids into adenovirus particles in order to serve as chemoselective attachment points. The inventors have shown that production of metabolically labeled virion is exceedingly straightforward, requiring only addition of the unnatural amino acid to virus production medium. Viral particles bearing these unnatural azido amino acids have no impact on either particle production or infectivity. Importantly, the installed azides afford access to several highly selective chemistries, which were used to attach small molecules and peptides. In order to demonstrate the utility of this approach, the inventors attached a cancer-targeting motif, folate, to hAd5. Folate decorated particles mediated gene delivery to breast cancer cells that are normally refractive to hAd5 infection.

Incorporation of Aha is accomplished via production of hAd5 under completely standard conditions with one exception; producing cells are shifted from normal to methionine-free/+Aha media between 18-24 hours post-infection. Despite previous reports demonstrating an absence of toxicity when Aha is used to label mammalian cellular proteins, the inventors chose to metabolically label hAd5 during a relatively brief 6 hour window to avoid potential complications stemming from Aha incorporation into cellular proteins. Chemical modification of the resultant particles and quantitation reveals >500 dye molecules/virion, when viruses are produced with 32 mM Aha. Notably, Aha produced no apparent effects on viral or cellular physiology even at concentrations above saturation. Although initially counterintuitive, Aha is more similar to Met in both sterics and electrostatics that it would appear from 2-dimensional drawings(10). Although expanding the Aha labeling window is likely to lead to higher unnatural amino acid incorporation, 500 modifications/particle is more than sufficient for most envisioned applications.

Chemical treatment, via either the Staudinger or Cu (I) catalyzed “click” ligations, yielded modified, infective viral particles. Selectivity, in this context, can be broken down into each process, metabolic labeling during viral production and chemical modification of the purified virion. Both CuAAC and Staudinger ligation demonstrate excellent selectivity (FIG. 3, 4). No modification is evident when Aha is not present, the copper is excluded or an inactivated (oxidized) Staudinger probe is used. This selectivity is independent of the nature of attached probe (peptide epitope or fluorophore). Incorporation of Aha is presumed to take place at each methionine codon independent of local sequence or structure. In support of this supposition, CuAAC mediated fluorescent labeling of full viral particles tracks with the number of methionine positions expected to be solvent exposed (FIG. 4C, Table 2). Mass spectral analysis of metabolically labeled hAd5 tryptic peptides demonstrates differences in the ratio of Met to Aha labeled peptides dependent upon the peptide analyzed. However, differences in ionization potential make this analysis difficult to quantitate and limit the value of these data.

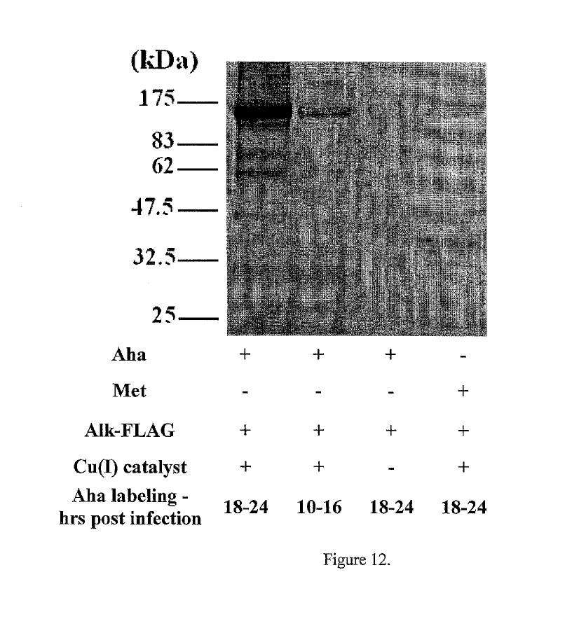

Interestingly, shifting the Aha labeling closer to infection (10-16 hours) shifts the labeling pattern (FIG. 12). Presumably, this is due to the staggered production of adenoviral structural proteins. Exploitation of such pulse-chase labeling could allow biasing of Aha labeling towards one structural protein or another. However, due to the largely overlapping nature of Ad structural protein production any potential biasing would be limited. Alternatively, replacing methionine codons with those of structurally similar amino acids allows the restriction and control of Aha mediated chemistry. Ideally this will allow the production of programmable viral particles, were chemoselective handles can placed site specifically by design. Precedence for this approach can be found with the removal of surface exposed aspargines, which hydrolize upon storage, changing surface charge and physiological behaviour of adenoviral vectors (1). A general lack of conservation for these exposed methionine codons indicates that this strategy will likely be successful.

“Click” reagents are easier to access as many are commercially available and terminal alkynes are relatively simple to synthesize. However, simple desalting via size exclusion chromatography, while effective at removing the alkyne reagent, is insufficient to remove the copper catalyst. In these cases, a significant drop in infectivity was observed (2-3 fold). Cu (I) can be efficiently removed via treatment with a high affinity chelator (bicinchoninic acid (BCA) followed by size exclusion chromatography. Alternatively, the Staudinger ligation or newly developed strain promoted “click” chemistry may be utilized. At present, reagents for both of these chemistries are more difficult to access than the terminal alkynes required for CuAAC. However, with the growing popularity of the copper-free strain promoted “click” chemistry, commercial reagents may soon be available.

The inventors have demonstrated targeting potential of Aha-enabled hAd5 via the straightforward attachment of well-utilized cancer specific ligand, folate. Although neither the physical nature nor linker length was optimized, folate decorated particles demonstrated a marked increase in infection of murine breast cancer cells (4T1), which are naturally refractive to infection. Although competition experiments indicate the folate receptor as the primary target for folate modified hAd5, the inventors do not know whether secondary interactions (i.e. integrin) are required or whether direct internalization, via the FR, is the dominant entry method. No difference in folate-mediated infectivity was found with particles produced in the presence of 4 mM or 32 mM Aha, indicating that 250 ligands per particle is saturating with respect to infectivity. Given the relatively small nature of the conjugate, it is possible that only a subset of these modification sites, potentially Aha sites on the fiber knob, are necessary to mediate infection.

Given both its completely unoptimized nature, the significant increase in infection seen as the result of conjugation, and the generality of the chemistry the inventors believe this platform has broad potential. Azide specific chemistries, as a whole, are extremely selective. This is evident in their application to modify metabolically labeled unnatural post-translational modifications such as oligosaccharides and lipids, which are often in very low concentrations relative to the proteome. As a result of the demonstrated specificity and tolerance towards essentially any other functionality, Aha-enabled adenoviral particles should be amenable to modification with an extremely wide variety of targeting ligands. Further, given the standard nature of these chemistries, characterization after modification may not be necessary, which would enable higher throughput screening of potential targeting ligands. The inventors believe that this flexibility, in combination with the ease of implementation, make this method a significant addition to the currently available methods for capsid remodeling.

In conclusion, the inventors have developed a novel strategy for the chemical modification adenoviruses. It relies on a two-step strategy that takes advantage of the natural fidelity of protein synthesis and the most selective bioorthogonal reactions described to date. As a result, this method demonstrates an unprecedented level of chemo selectivity. The high level of control limits impact on viral fitness and significantly expands the breadth of targeting and imaging moieties that can be approached. Despite these advantages, modification is remarkably straightforward, with minor modifications to standard Ad production protocols and requiring only widely available chemical reagents. Given the standard nature of these chemistries, characterization after modification may not be necessary, which would enable higher throughput screening of potential targeting ligands. Further, this method is not limited to adenoviruses, but is expected to be robust when used in conjunction with most, if not all, viruses. The inventors believe that this flexibility, in combination with the ease of implementation, make this method a significant addition to the currently available methods for capsid remodeling.

### Example 8

### Materials and Methods Used in Examples 9-14

Reagents and Equipment

All chemical reagents were obtained from commercial sources and used without further purification unless otherwise noted. NMR spectra were recorded on a Varian 300 NMR spectrometer. Mass spectra for the small molecules were obtained using an Agilent 1100 LC/MSD VL instrument. The α-FLAG M2 conjugate was obtained from Sigma (St. Louis, Mo.). Centri Sep spin columns were obtained from Princeton Separations (Adelphia, N.J.). RP-HPLC was performed using a L201243 Shimadzu on a C18 Jupiter column (250×10 mm; Phenomenex). Mouse breast cancer cell line, 4T1 was obtained from ATCC (Manassas, Va.). UV-Visible absorbance was recorded on a Beckmann Coulter DU 730. Electrophoresis gels were scanned on a Typhoon 9400 fluorescent gel scanner and GFP expression read on a Synergy 2 fluorescent plate reader.

Synthesis

1,3,4,6-tetra-O-acetyl-N-azidiacetyl-α,β-D-glucosamine and 1,3,4,6-tetra-β-acetyl-N-azidoacetyl-α,β-D-galactosamine. The peracetylated sugars were synthesized exactly as described by Bertozzi¹ et al.

Alkyne-Folate

Pentynoic acid was activated by DCC and NHS at a 1:2:2 molar ratio in methylene chloride at RT for 12 hours and filtered with a syringe filter (pore size 0.2 μm). O—(N-Trt-3-aminopropyl)-O′-(3-aminpropyl)-diethyleneglycol (MW 462) was added to the solution at a molar ratio of 5:1 (alkyne:PEG) and stirred vigorously overnight. The reaction was acidified using 1% TFA to remove the protecting trityl group. The reaction was neutralized with TEA and concentrated on high vacuum. Folate activated by NHS and DCC was added to the Alkyne-PEG under an atmosphere of argon in DMSO and allowed to react for 12 hours. The alkyne-folate was precipitated from the reaction mixture by repeated ether precipitation and dissolution in DMSO. The crude product was purified on a Jupiter C-18 column with a gradient of 5% to 25% acetonitrile containing 0.1% TFA, the product eluting after 30 minutes. ¹H NMR (300Mz, D₂O): δ 1.07 (2H, t), 1.10 (4H, t), 1.89 (4H, m), 1.92 (12H, m), 2.71 (1H, s), 2.87 (1H, s), 3.4 (2H, m), 3.50-3.51 (2H, m), 3.97 (4H, q), 6.7 (2H, d), 7.5 (2H, d), 9.0 (1H, s) ppm. ESIMS calculated for C₃₄H₄₅O₉N₉ (M+H⁺) 724.3; found 724.3.

Alkyne- and Phosphine-FLAG

Synthesized as described by Bertozzi² et al. Briefly, all peptides were synthesized by standard Fmoc SPPS protocol using diisopropyl carbodiimide and HOBt to form activated esters. Alkyne functionalized peptides were produced by on-bead N-terminal functionalization with propynoic acid. Phosphine FLAG was obtained by on-bead N-terminal derivitization with 2-(Diphenylphosphino)terephthalic acid 1-methyl 4-pentafluorophenyl diester (Sigma-Aldrich 679011). During all deblocking steps 0.1 M HOBt was added to the 20% piperidine solution to alleviate aspartamide formation.

Cell Culture.

Dulbecco's modified Eagle's medium (DMEM), RPMI 1640, Penicillin/Streptomycin and 0.5% Trypsin-EDTA were purchased from GIBCO (Grand Island, N.Y.). RPMI 1640 minus Folic acid was obtained from Sigma (St. Louis, Mo.). Fetal calf serum (FCS) and Bovine Calf Serum (BCS) was from HyClone (Logan, Utah). 293 cells were maintained in DMEM supplemented with 10% BCS, 2 mM glutamine, 100 U/mL penicillin, 100 μg/mL streptomycin. 4T1 cells were maintained in RPMI 1640 medium containing 10% FCS, 2 mM L-glutamine, 100 U/mL penicillin, and 100 μg/mL streptomycin. For 4T1 cells grown without Folate, RPMI 1640 minus Folic acid was supplemented with 10% FCS, 0.3 mg/mL L-glutamine, 2 mg/mL sodium bicarbonate, 100 U/mL penicillin, 100 μg/mL streptomycin. All cells were maintained in 100×20 mm Tissue Culture Dishes obtained from BD biosciences (Franklin Lakes, N.J.) at 37° C. and 5% CO₂.

Metabolic Labeling of Adenovirus Type 5 with N-azidoacetygalactosamine.

HEK 293 cells were infected with wild type adenovirus particles with an MOI of 5 pfu/cell. The complete media was supplemented with 50 μM peracetyl-N-azidoacetygalactosamine or 50 μM peracetyl-N-azidoacetyglucosamine and the infected cells incubated at 37° C. The plates were harvested 42-46 hours post infection and virus particles purified over a gradient of 1.4 g/mL and 1.25 g/mL CsCl centrifuged at 32,000 rpm for 1 hour at 15° C. The virus band at the junction of the two CsCl bands was collected and further purified by an 18 hour centrifugation at 35,000 rpm over 1.33 g/mL of CsCl.

Reaction of Azide Labeled Virus with Alkyne-FLAG.

50 μL of 1×10¹² azide labeled virus particles/mL in a 100 mM tris buffer pH 8.0 was mixed with Bathophenanthroline disulphonic acid disodium salt at a final concentration of 3 mM and alkyne-FLAG at a final concentration of 400 μM were kept in a deoxygenated glove bag for 6 hours after which copper bromide was added to the mixture at a final concentration of 1 mM and the reaction allowed to proceed for 12 hours³ inside the glove bag. The samples were then taken out and the reaction quenched by 10 mM EDTA. The samples were then analyzed by western blotting technique to determine incorporation of FLAG on the viral proteins.

Reaction of Azide Labeled Virus with Phosphine-FLAG.

50 μL of 1×10¹² azide labeled viral particles/mL in a 100 mM tris buffer at pH 8.0 was treated with phosphine-FLAG at a final concentration of 400 μM in room temperature for 2 hours and then analyzed by western blotting.

Western Blotting.

To all samples coupled with alkyne- and phosphine-FLAG loading dye was added and phosphine-FLAG labeled samples boiled at 95° C. for 10 minutes. The samples were run on a 10% polyacryamide electrophoresis gel and transferred onto nitrocellulose at 40V over 2 hours in a western transfer buffer (25 mM tris, 192 mM glycine, 0.5% SDS and 10% methanol). Blots were blocked by 5% milk in PBST and treated with anti-FLAG M2 HRP conjugate at a ratio of 1:12000 in 5% milk in PBST. Blots were washed with milk and PBST and developed by chemiluminescence (Millipore Immobilon Western kit).

GlcNAcase Assay.

For treatment with GlcNAcase, adenoviral fiber was partially purified from complete virus particles. GlcNAz labeled adenoviral particles were dialyzed overnight in a tris-maleate (5 mM tris, 5 mM maleic acid, 1 mM EDTA) buffer at pH 6.5. The dialyzed solution was centrifuged at 14.2 rpm for 60 minutes after which the supernatant containing the Penton and Fiber proteins were separated from the remaining viral capsid which precipitates. The mixture of Fiber and Penton was treated with 5M guanidinium hydrochloride and subjected to acetone precipitation. The protein was resuspended in 50 mM tris, 12.5 mM MgCl₂ at pH 7.5 to a concentration of 0.5 mg/mL after which it was treated with 5 μg/μL Hex-C at 1:10 (Fiber:Hex-C) overnight at 37° C.⁴. The reaction mixture was subjected to Staudinger reaction with phosphine-FLAG for 2 hours and analyzed by western blotting as described above.

Plaque Assay.

HEK 293 cells cultivated in 6 mL culture plates were infected with 400 μL of azide labeled virus particles at concentrations of 10³ particles/ml. The plates were overlaid with complete media containing 2.8% bactoagar and 10% BCS. After 3 days the cells were again overlaid with same agar solution containing 2% BCS. After the 6^{th} day when plaques became visible the cells were overlaid with agar solution containing 0.1% neutral red. The plaques were counted within a day after the final overlay.

Fluorescent Gel Scanning Assay.

Azide enabled viral particles (with 50 μM GalNAz) were labeled with an alkyne-TAMRA dye using Cu catalyzed “Click” chemistry as described above for FLAG labeling and reaction was quenched with 10 mM EDTA. The particles were purified using Centri-Sep spin columns and quantified with QuantIT Picogreen Dye labeling⁵. 1×10¹² viral particles/mL were run on a polyacrylamide electrophoresis gel using the alkyne-TAMRA dye as standard. Standard dye was loaded 10 minutes before the end of the run. Gels were scanned using a typhoon gel scanner in the fluorescence mode with excitation filter at 532 nm and emission filter at 580±15 nm. The scans were subsequently analyzed with Image Quant TL 1D gel analyzer software. All gels were run at 4° C. for 60 minutes and scanned within 10 minutes of the end of run.

Targeting Assay.

GFP transgene bearing Ad5 were labeled with GalNAz as described. The viruses were labeled with folate bearing alkyne probe (alkyne-folate) using Cu catalyzed “click” chemistry in a deoxygenated glove under same conditions (as for FLAG labeling) and the reaction quenched with 10 mM EDTA. Viruses were purified on Centri Sep spin columns and quantified using QuantIT Picogreen assay (Molecular Probes, Eugene, Oreg.) and stored in a 0.9 mM CaCl₂ and 0.5 mM MgCl₂ buffer in PBS containing 10% glycerol. Mouse breast cancer cell line 4T1 was cultivated in minus folate media for 2 weeks after which they were seeded in 24 well plates at a density of 1×10⁶ cells/well and cultivated for a day in minus folate media containing 2% FCS. After 24 hours the cells were infected with labeled virus at an MOI of 50. 24 hours post infection; GFP expression was evaluated using a Synergy 2 fluorescence plate reader (excitation 485±10 nm; emission 528±10 nm).

Fluorescence Microscopy:

For visualization of GFP expression within targeted 4T1 cells, the murine breast carcinoma cells grown in minus folate media for 2 weeks were plated on Glass Bottom Dishes at a density of 1×10⁵ cells/well and cultivated for 24 hours in negative folate media. After which time they were infected with folate-labeled virus, unlabeled virus and folate-labeled virus but with cells pretreated with 1 mg/L folate 30 minutes before infection at an MOI of 50. Again 24 hours post infection the cells were visualized under a Zeiss LSM 510 META NLO 2-photon laser scanning confocal microscope system. Images were taken both with a GFP and the bright fields filter.

### Example 9

### Producing Modified Adenovirus Having Azido Sugar Modifications

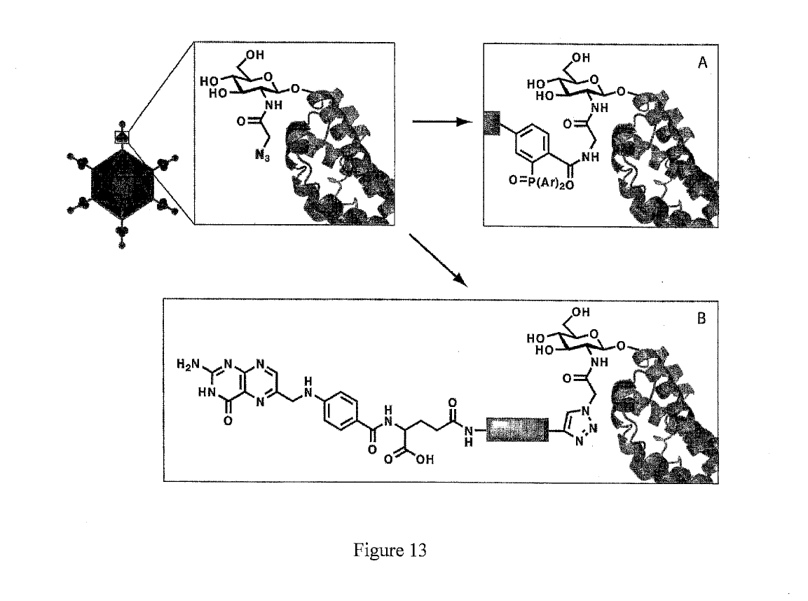

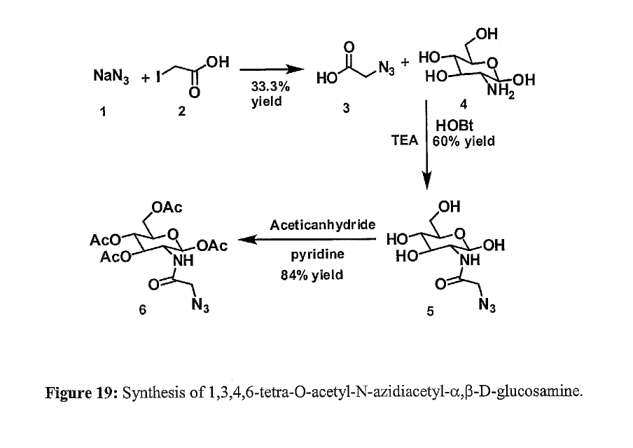

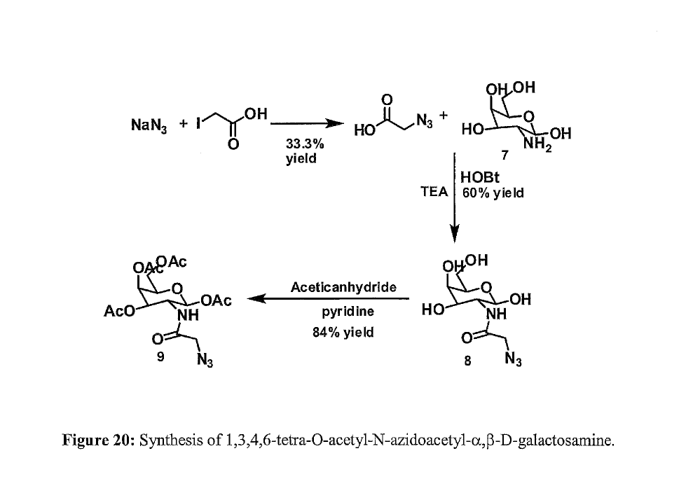

Human Ad5 is reported to have a single O-GlcNAc residue on Ser-109 of the fiber protein (FIG. 13), which exists as a homotrimer on twelve vertices of the virus capsid.^{17,18} Replacing this sugar residue with an azido analog would allow for specific placement of an azide on the viral coat. Advantageously, Ser-109 is located proximal to the natural primary targeting motif on the fiber knob. Thus chemoselective placement of targeting moieties at this site is expected to be effective for re-directing viral particles. Towards this goal, E1 deleted Ad5 particles were propagated on HEK 293 cells. During the infection period, the culture media was supplemented with 50 μM Ac₄GalNAz (FIG. 19) or Ac₄GlcNAz (FIG. 20). Both precursor sugars are metabolic precursors of UDP-GlcNAz, a known substrate for O-GlcNAc Transferase.¹⁹ Viruses produced from the infected 293 cells were purified on double CsCl gradients and probed for incorporation of azido sugar with an engineered peptide epitope FLAG (DYKDDDDK), bearing either an N-terminal alkyne or a modified triaryl phosphine motif (FIG. 13).

### Example 10

### Determining Chemical Accessibiliy of Unatural Sugars on Modified hAd5

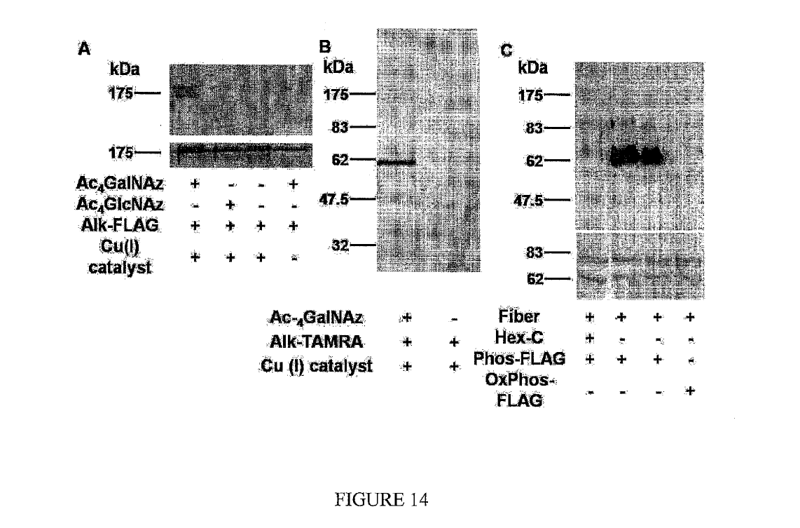

Purified viruses were modified with FLAG epitope and tetramethylrhodamine (TAMRA) probes using the copper accelerated azide-alkyne cycloaddition (CuAAC),^{20,21} commonly known as the “click” chemistry, or the Staudinger ligation reaction.^{22,23} Initial characterization of the O-GlcNAc site of hAd5 indicated that it was enzymatically inaccessible in complete virion. In order to determine if an installed O-GlcNAz was chemically accessible, whole viruses were chemically treated, and not denatured, prior to analysis. While western analysis of viruses grown with either Ac₄GalNAz or Ac₄GlcNaz sugar demonstrated effective labeling of the fiber protein (MW 61.5 kD) with O-GlcNAz (FIG. 14A), Ac₄GalNAz mediated labeling was markedly higher. In order to confirm both the identity and placement of the installed azide, modification analysis of both denatured virus and partially purified fiber was carried out. Modification of the denatured virus with a fluorescent probe under click conditions demonstrated exclusive labeling of a protein at 62 kD (FIG. 14B). This corresponds to either fiber or protein Ilk, which co-migrate. Fiber was partially purified from complete virus and treated with a hexosaminidase known to remove both O-GlcNAc and O-GlcNAz.¹⁹ Subsequent chemical modification demonstrated almost a complete loss of azide-dependent signal (FIG. 14C). Cumulatively, these results strongly indicate the fiber bears an O-GlcNAz modification, which is the exclusive source of virus labeling.

### Example 11

### Modified Adenovirus Having Azido Sugar Modifications Had Little Impact on hAd5 Production and Infectivity and on Producer Cell Viability

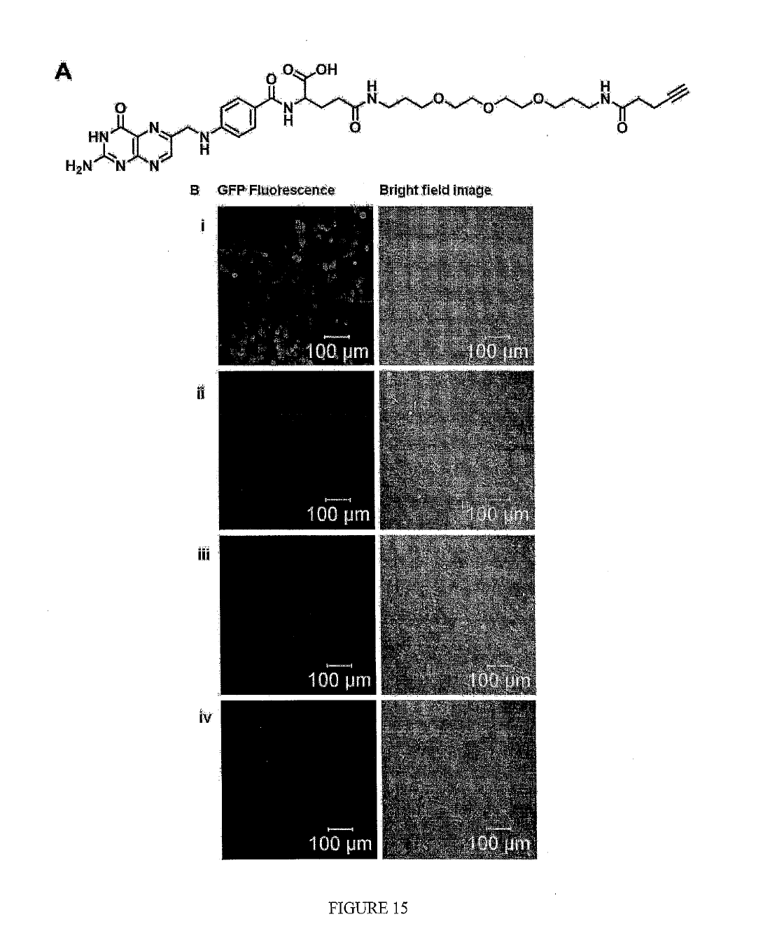

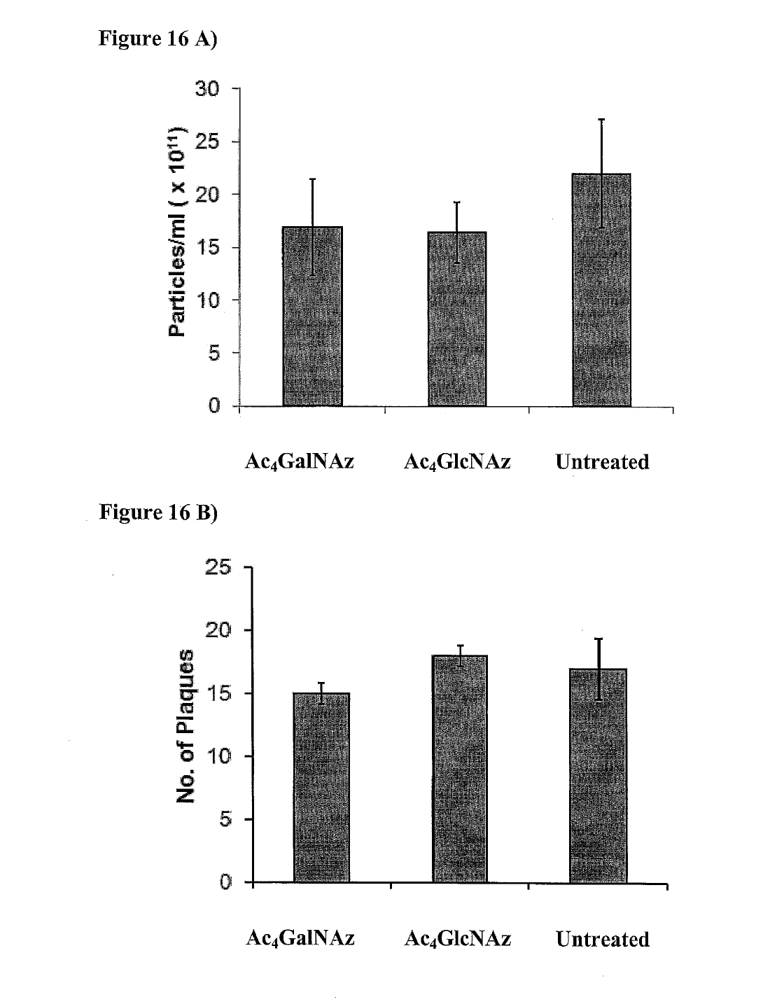

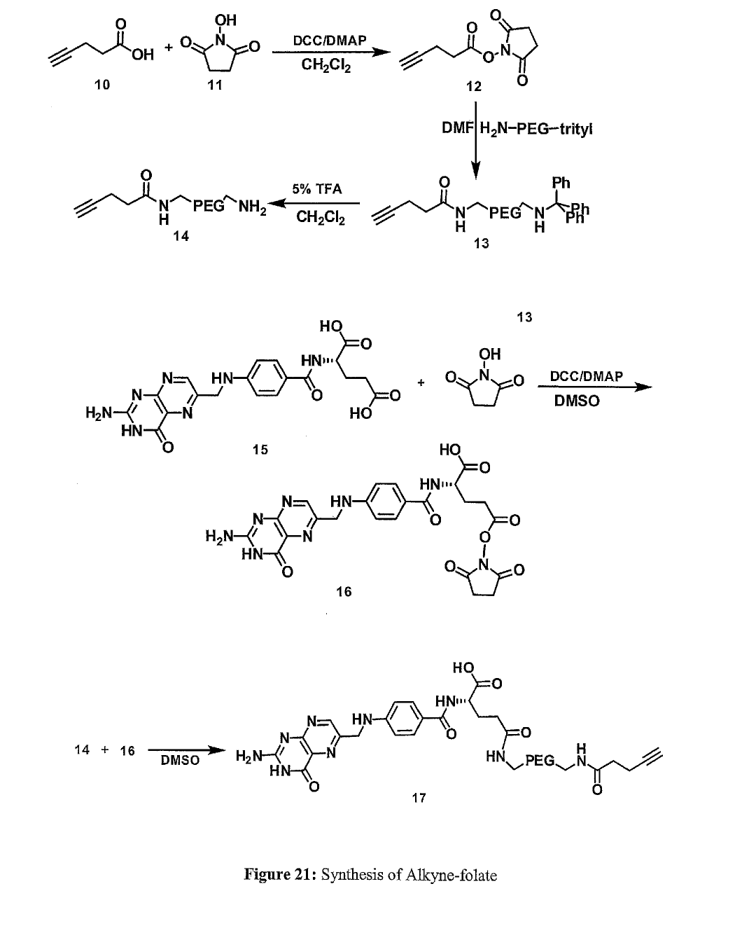

In contrast to genetic engineering, the introduction of O-GlcNAz had little impact on adenoviral physiology. Virus production in the presence of either azido sugar demonstrated no difference on producer cell viability. In addition, no reduction in particle production was apparent as a result of FIG. 15. Effective gene transduction of 4T1 cells with retargeted Ad5. A) Structure of alkyne-folate ligand. See FIG. 21. B) GFP fluorescence microscopy images of alkyne-folate modified metabolically labeled Ad5 virus and metabolically unlabeled Ad5 infected 4T1 cells. Microscopy was carried out on Glass Bottom dishes using a Zeiss LSM 510 fluorescence microscope. Cells were infected at an MOI of 50 pfu/cell and images taken 24 hours post infection. (lanes i: 4T1 cells infected with folate modified Ac₄GalNAz labeled Ad5; lane ii: 4T1 cells infected with metabolically unlabeled Ad5 (no Ac₄GalNAz); lane iii: 4T1 cells infected with folate unmodified Ac₄GalNAz labeled Ad5 (no alkyne-folate); lane iv: 4T1 cells infected with folate modified Ac₄GalNAz labeled Ad5 pre-treated with 1 mg/L folic acid). Azide incorporation (FIG. 16A). Similarly, infectivity of O-GlcNAz labeled virus particles as assessed via standard plaque forming assay, remained unaffected (FIG. 16B).

### Example 12

### Determination of Checmically Available Azides on Modified hAd5

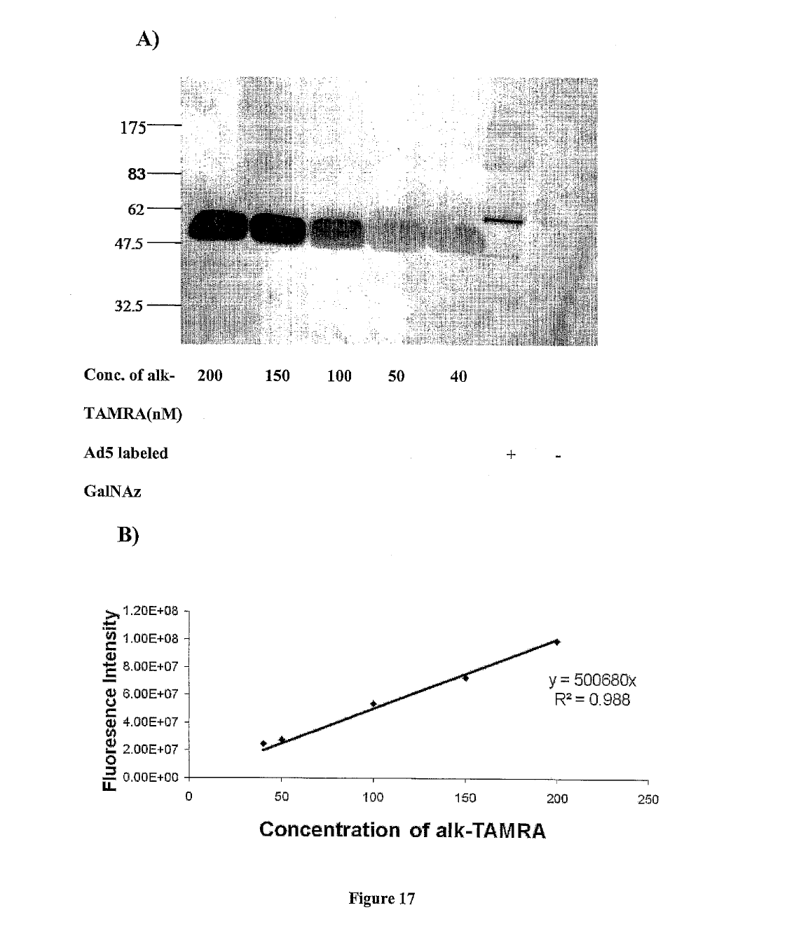

Fluorescently labeled particles were analyzed to determine the number of chemically addressable azides on the viral surface. Quantification of the number of on each virus by fluorescent gel scanning demonstrated exclusive fiber labeling in agreement with Western analysis (FIG. 17) and an average attachment of 21.9±1.5 dyes per particle, consistent with previous O-GlcNAc characterization indicating ˜50% occupancy at Ser109.¹⁸

### Example 13

### Prodution of Folate Modified Adenovirus by Conjugating O-GlcNA Modified hAd5 to Alkyne Folate

Low CAR expression is exhibited in a number of cancers, including ovarian, pancreatic and gastrointestinal cancers, generally limiting the effectiveness of hAd5 for oncolysis.²⁴⁻²⁷ As a result of tumor associated overexpression and limited abundance in normal tissues, the folate receptor has become an appealing target for cancer therapy.^{28,29} In addition, folate conjugates are easily produced and generally have minor impact on receptor affinity. In order to explore O-GlcNAz mediated folate modification the inventors synthesized an alkyne probe containing a folic acid residue tethered by a small linker (FIG. 15A and FIG. 21). O-GlcNAz enabled adenovirus, encoding a GFP transgene in the E1 deleted region of the viral genome regulated by a CMV promoter, was conjugated to alkyne-folate via CuAAC.

### Example 14

### Treatment In Vivo of Breast Carccinoma Cells with Folate-Modified hAd5

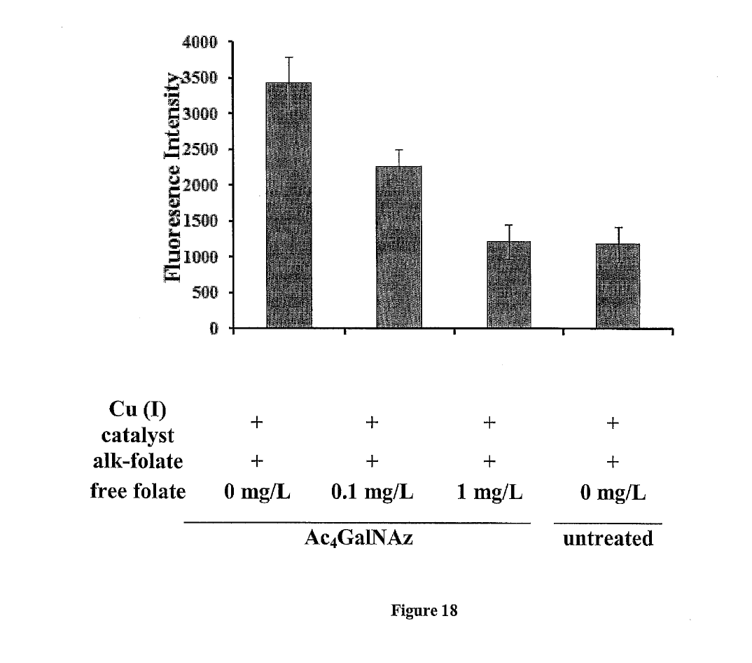

Mouse breast carcinoma cells (4T1), known to express moderate to high levels of folate receptors³⁰ and naturally refractive to human adenovirus, were cultured in folate deficient media for 2 weeks. 24 hours after replating, the cells were infected with folate decorated virus at an MOI of 50. One day after infection the GFP expression was visualized. The results demonstrate a high level of GFP expression within the 4T1 cells infected with the folate labeled virus (FIG. 15B). Human Ad5 produced without azido sugar, but treated with the folate reagent under CuAAC conditions, failed to produce significant transgene expression. In addition, free folate completely abrogated infection of folate modified hAd5, indicating folate receptor meditated gene delivery. Quantification of infection was assessed using a Synergy 2 fluoresence plate reader (excitation 485±10 nm; emission 528±10 nm) which showed a 3-4 fold increase of GFP expression in cells infected with metabolically and chemically modified virions versus unmodified virus. Infection assayed in the presence of increasing free folate concentration demonstrated dose dependent transfection inhibition of 4T1 cells (FIG. 18).

In summary the inventors have demonstrated that adenoviruses can be chemoselectively labeled through a two-step process. Metabolic labeling with azido sugars yields adenoviral particles with site-specific placement of a chemically accessible azide without loss in either viral production or infectivity. Subsequent chemical modification of these particles allows the facile appendage of a variety of functionality from peptides to fluorophores to small molecules targeting moieties. The remarkable ease and specificity of this approach in combination with its non-perturbing nature make it accessible to a wide range of researchers. Further, while the broad application of adenoviral vectors make them particularly attractive targets, this approach is not limited to adenoviruses, but is expected to be generally applicable to the wide variety of viruses with peripheral glycoproteins, including many oncolytic vectors currently under development (retroviruses, lentiviruses, poxviruses and herpes viruses).

### Example 15

### Targeted and Armed Oncolytic Adenovirus Via Chemoselective Modification

Oncolytic adenoviruses (Ads) are an emerging alternative therapy for cancer; however, clinical trial have not yet demonstrated sufficient efficacy. When oncolytic Ads are used in combination with taxoids a synergistic increase in both cytotoxicity and viral replication is observed. In order to generate a next generation oncolytic adenovirus, viral particles were physically conjugated to a highly potent taxoid, SB-T-1214, and a folate targeting motif. Conjugation was enabled via the metabolic incorporation of non-canonical monosaccharides (O-GlcNAz) and amino acids (homopropargylglycine), which served as sites for chemoselective modification.

Despite substantial progress in understanding the molecular underpinnings of cancer, current chemotherapeutic options are limited and often unsuccessful. On promising alternative strategy is the use of conditionally replicative oncolytic vectors. Such viruses are designed to preferentially replicate in cancerous cells, such as those lacking common tumor suppressors (e.g. p53), leading to partially selective tumor toxicity. In addition, many carry a toxic transgene designed to amplify the inherent cytotoxic nature, which results from viral protein expression and immune stimulation.¹ Despite this multifaceted cytotoxicity, the major limitation for oncolytic viruses in clinical trials has been efficacy.² In an effort to increase potency, a number of oncolytic viruses have been used in combination with traditional chemotherapeutics.³⁻⁵ In particular, conditionally replicative adenoviruses (Ads) have demonstrated significant synergism when used in combination with a number of different chemotherapeutics including doxorubicin, paclitaxel/docetaxel, cisplatin and histone deacetylase inhibitors.⁶ In the case of taxoid/oncolytic Ad combination therapy, an increase in viral replication is seen in addition to synergistic cytotoxicity.⁷⁻¹² While the mechanistic origin of synergism is not well understood, it is clearly a general and significant phenomenon.

Paclitaxel treatment of cancer cells results in upregulation of TNF related apoptosis inducing ligand (TRAIL) receptors.¹³ Notably, one of the most promising oncolytic Ads in clinical trials bears the cytotoxic TRAIL transgene, which induces apoptosis in the infected cell and mediates substantial bystander cytotoxicity. As a result, taxoid/AdTRAIL would be expected to have an additional source of synergism. SB-T-1214 is a next generation taxoid that exhibits significantly improved cytotoxicity, against a number of drug resistant cancer cell lines.¹⁴⁻¹⁶ In addition, this taxoid exhibited substantial inhibition of cancer stem cell related genes (Oct4, Sox2, Nanog, and c-Myc) when screened against 3 unrelated invasive colon cancer cell lines.¹⁷ These results indicate that SB-T-1214 has significant potential, particularly with respect to cancer stem cells and cancers that are resistant to traditional chemotherapeutics.

While combination therapy demonstrates significant promise, it holds that efficiently targeted Ad particles bearing a therapeutic payload would provide an additional boost in efficacy. This would be a result of spatially and temporally concerted delivery of cytotoxicity, and may have the added benefit of reducing systemic toxicity. In order to achieve this goal, selective chemical modification routes for adenovirus are required, particularly those that allow the generation of multifunctional particles. Previously we reported the incorporation and modification of a non-canonical sugar residue, O-GlcNAz on serine 109 of the fiber protein, as a means of chemoselectively tailoring Ad particles.¹⁸ The specificity of this strategy, derived from the fidelity of the biosynthetic machinery and the highly selective chemistries developed for azide modification, allowed folate modification without compromising virus infectivity. Folate decorated Ad exhibited substantial (˜20 fold) increase transgene delivery to breast cancer cells.¹⁸

Here, we extend this approach towards multimodal adenovirus particles via the simultaneous metabolic labeling with O-GlcNAz and an alkyne bearing non-canonical amino acid, homopropargylglycine (HPG). Introduction of these surrogates into Ad particles was envisioned to allow sequential Staudinger ligation of O-GlcNAz followed by copper assisted “click” modification of homopropargylglycine (HPG).

In order to explore the potential of this approach, adenovirus type 5 particles were produced in the presence of a metabolic precursor of GlcNAz, peracetylated N-azidoacetylgalactosamine (Ac₄GalNAz), and HPG.^{19,20} Azido-sugar incorporation was accomplished by supplementing media with 50 μM Ac₄GalNAz for the entire duration of virus production (48 hours). Introduction of the alkyne-amino acid was mediated by exposure of producer cells to 4 mM HPG during a six-hour window (18-24 hours post infection), in a pulse chase format with methionine containing media. 48 hours post infection, the cells were harvested, lysed and viruses were purified via a standard two-step ultracentrifugation procedure in CsCl gradients.²¹

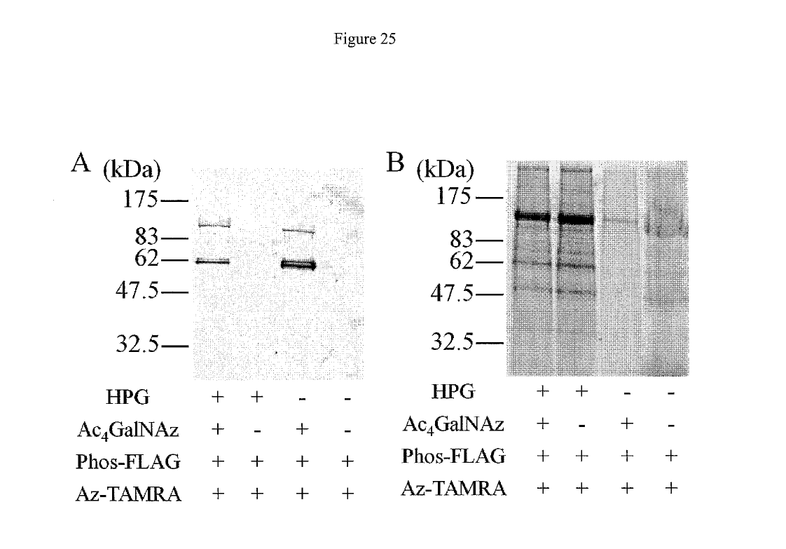

Purified O-GlcNAc and HPG bearing viruses (HGP/GlcNAz-Ad) were treated with 300 μM of Staudinger probe bearing a FLAG epitope (PhosFLAG) (3 hours, RT).²² Reaction mixtures were subsequently exposed to tetramethylrhodamine 5-carboxamido-(6-azidohexanyl) (az-TAMRA) dye (500 μM) using copper assisted “click” reaction conditions under de-oxygenating conditions in the presence of bathophenanthroline disodium salt (3 mM) and CuBr (1 mM) (RT, 12 hours).²³ Particles were purified by size exclusion (Sephadex G-25) and interrogated by western blot and fluorescent gel imaging. Western analysis demonstrated that HGP/GlcNAz-Ad and GlcNAz-Ad are labeled on a single coat protein occurring at 62 kD by PhosFLAG (FIG. 25a). No PhosFLAG labeling is seen on particles produced in the absence of Ac₄GalNAz, consistent with previous studies demonstrating the specific labeling of the fiber protein via O-GlcNAz. Fluorescent gel imaging of az-TAMRA labeled HPG-Ad and HPG/GlcNAz-Ad demonstrated labeling of a number of different surface exposed proteins, consistent with the presence of solvent exposed methionine sites (FIG. 25b).²⁴

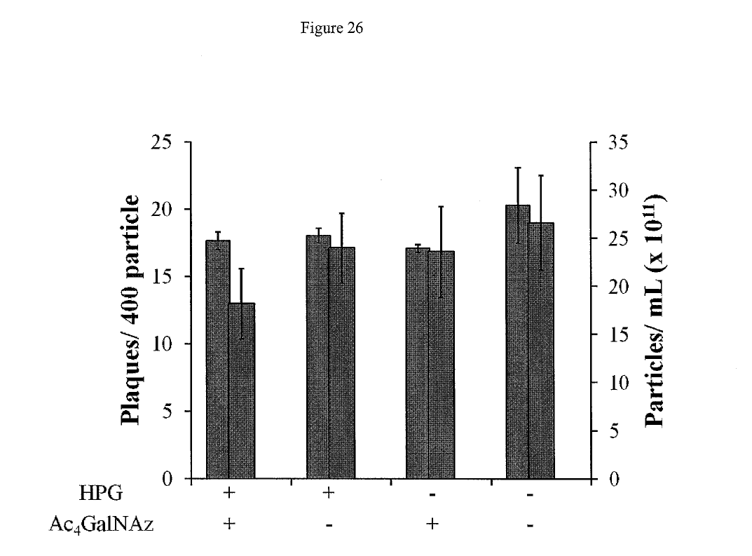

Previous characterization of GlcNAz-Ad particles demonstrated 22±1.5 chemically addressable azides per particle.¹⁸ In order to quantitate HPG incorporation, az-TAMRA labeled HPG-Ad viruses were quantified via fluorescent gel imaging against a free az-TAMRA standard addition curve, demonstrating an attachment of 193±12 dyes per particle (supplementary table 1). Vector production and infectivity are often compromised during genetic engineering of Ad particles, which has slowed the pace of vector development and effectively limited the production of multifunctional particles. While previous results indicate that O-GlcNAz incorporation does not impact either particle production or infectivity, the incorporation of HPG into the protein backbone at significantly higher incorporation levels was a concern. Surprisingly, no significant loss in either particle production or infectivity was observable for either of the singly modified Ad particles (GlcNAz-Ad; HPG-Ad) or particle bearing both O-GlcNAz and HPG (HPG/GlcNAz-Ad) (FIG. 26).

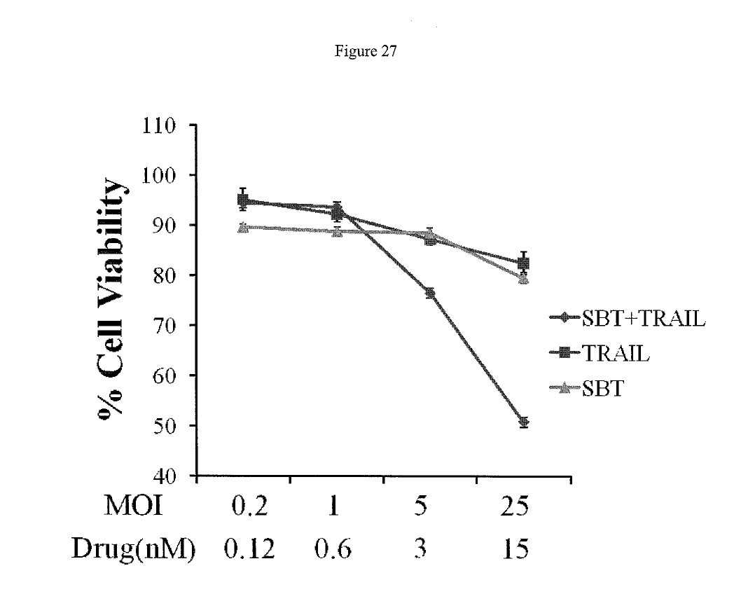

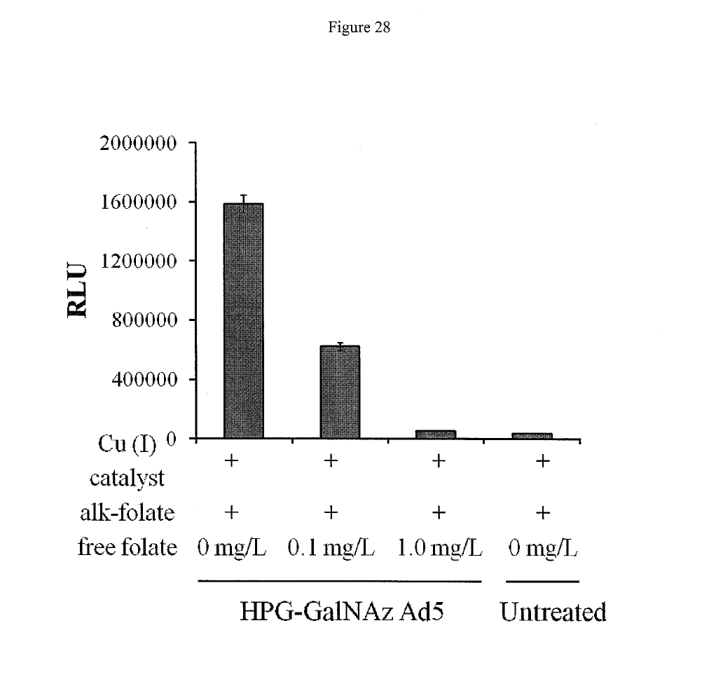

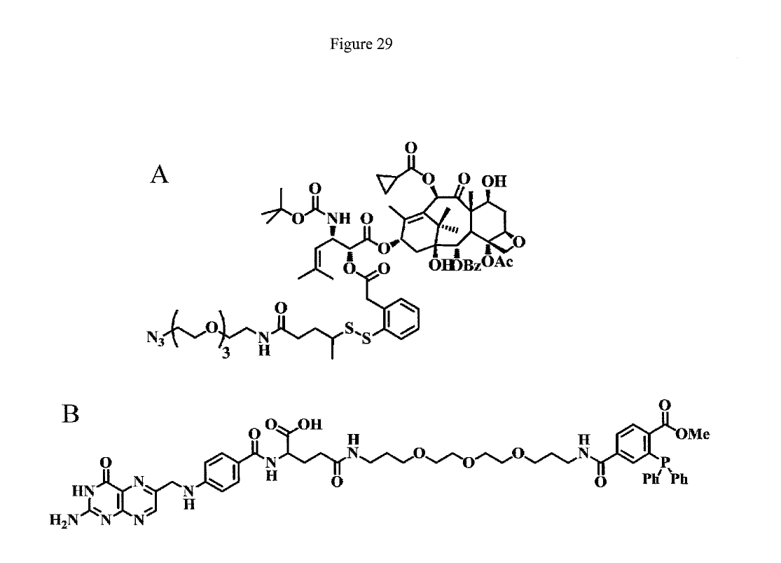

In order to generate a chemotherapeutically “armed” Ad particle, an azido derivative of SB-T-1214 (az-SB-T-1214) was synthesized. This molecule includes a reductively self-immolative linker, designed to release the taxoid ofter Ad particle endocytosis (Scheme 1b). Modification of HPG/GlcNAz-AdTRAIL with az-SB-T-1214 was accomplished in an identical manner to az-TAMRA modification of HPG/GlcNAz-Ad described above. Chemically modified viruses (SBT-AdTRAIL) were purified by size exclusion and used to infect ovarian cancer cells, ID8, at mulitplicities of infection (MOIs) that were expected to be subtoxic for AdTRAIL alone. Levels of free SB-T-1214 equivalent to that loaded on the “armed” viruses, as well as unmodified AdTRAIL were used for comparison Importantly, the only difference between the processing of the “armed” SBT-AdTRAIL and “unarmed” AdTRAIL was the addition of HPG during production of the former. Specifically, AdTRAIL was exposed to identical “click” conditions as SBT-AdTRAIL, however due to the absence of HPG was presumably unmodified. Five days post infection, cytotoxicity was assessed via MTT assay (Roche, KitI). SBT-AdTRAIL demonstrated an increase in cytotoxicity compared to free SB-T-1214 and AdTRAIL alone (FIG. 27), consistent with the proposed synergistic effect.

As many cancers demonstrate significantly higher levels of folate receptor (FR), folate conjugates demonstrate selectivity for this receptor and folate conjugates are efficiently internalized, folate has been widely used for cancer targeting. Although we have previously the gene delivery of folate-Ad particles, those modified with both az-SB-T-1214 and Phos-folate may demonstrate altered uptake profiles. In order to explore these effects, dually modified Ad bearing a luciferase transgene (AdLuc) were screened against a murine breast cancer cell line, 4T1.²⁵ Cells grown under folate free media for 2 weeks were seeded on 96 well plates at concentrations of 1×10⁴ cells/well. Infection was accomplished at a MOI of 50 and cells were examined for luciferase activity 24 hours post infection (Luc-Bright-Glow). Folate targeted SBT-AdLuc (folate/SBT-AdLuc) demonstrated (FIG. 27b) a ˜30 fold increase in transgene expression on breast cancer cell type (4T1) compared to viral particles lacking O-GlcNAz. In addition, treatment of cells with free folic acid prior to infection led to a dose dependent loss in virus infection (FIG. 27b).

In summary data herein demonstrate the development of a novel multi-functional adenoviral platform via non-canonical substrate incorporation and chemoselective modification. We utilized the platform to develop a combination vector targeted towards the folic receptor and armed with a next generation taxoid. The described system allowed the efficient modification with both functionalities without impact on viral fitness. Further, initial studies indicate significant synergistic cell toxicity. Ongoing studies will evaluate the construct in the context of different cancers, both in cell culture and within in vivo xenograft model systems. In principal the described dual modification methodology can be utilized to append targeting, imaging, diagnostic and chemotherapeutic modules to replication selective Ad, potentially accelerating vector development and allowing the evaluation of alternative combination therapies.

### REFERENCES IN BACKGROUND AND EXAMPLES 1-7

1. Blanche, F., B. Cameron, S. Somarriba, L. Maton, A. Barbot, and T. Guillemin. 2001. Stabilization of Recombinant Adenovirus: Site-Directed Mutagenesis of Key Asparagine Residues in the Hexon Protein. Analytical Biochemistry 297:1-9.
2. Campos, S. K., M. B. Parrott, and M. A. Barry. 2004. Avidin-based Targeting and Purification of a Protein IX-modified, Metabolically Biotinylated Adenoviral Vector. Mol Ther 9:942-954.
3. Douglas, J. T., B. E. Rogers, M. E. Rosenfeld, S. I. Michael, M. Feng, and D. T. Curiel. 1996. Targeted gene delivery by tropism-modified adenoviral vectors. Nat. Biotech. 14:1574-1578.
4. Fabry, C. M. S., M. Rosa-Calatrava, J. F. Conway, C. Zubieta, S. Cusack, R. W. H. Ruigrok, and G. Schoehn. 2005. A quasi-atomic model of human adenovirus type 5 capsid. EMBO J 24:1645-1654.
5. Fabry, C. M. S., M. Rosa-Calatrava, C. Moriscot, R. W. H. Ruigrok, P. Boulanger, and G. Schoehn. 2009. The C-Terminal Domains of Adenovirus Serotype 5 Protein IX Assemble into an Antiparallel Structure on the Facets of the Capsid. J. Virol. 83:1135-1139.
6. Glasgow, J. N., M. Everts, and D. T. Curiel. 2006. Transductional targeting of adenovirus vectors for gene therapy. Cancer Gene Ther. 13:830-844.
7. Gupta, S. S., J. Kuzelka, P. Singh, W. G. Lewis, M. Manchester, and M. G. Finn. 2005. Accelerated Bioorthogonal Conjugation: A Practical Method for the Ligation of Diverse Functional Molecules to a Polyvalent Virus Scaffold. Bioconj. Chem. 16:1572-1579.
8. Henning, P., E. Lundgren, M. Carlsson, K. Frykholm, J. Johannisson, M. K. Magnusson, E. Tang, L. Franqueville, S. S. Hong, L. Lindholm, and P. Boulanger. 2006. Adenovirus type 5 fiber knob domain has a critical role in fiber protein synthesis and encapsidation. J. Gen. Virol. 87:3151-3160.
9. Jung, Y., H.-J. Park, P.-H. Kim, J. Lee, W. Hyung, J. Yang, H. Ko, J.-H. Sohn, J.-H. Kim, Y.-M. Huh, C.-O. Yun, and S. Haam. 2007. Retargeting of adenoviral gene delivery via Herceptin-PEG-adenovirus conjugates to breast cancer cells. Journal of Controlled Release 123:164-171.
10. Kiick, K. L., E. Saxon, D. A. Tirrell, and C. R. Bertozzi. 2002. Incorporation of azides into recombinant proteins for chemoselective modification by the Staudinger ligation. Proc. Natl. Acad. Sci. USA 99:19-24.
11. Kreppel, F., J. Gackowski, E. Schmidt, and K. Stefan. 2005. Combined Genetic and Chemical Capsid Modifications Enable Flexible and Efficient De- and Retargeting of Adenovirus Vectors. Mol. Ther. 12:107-117.
12. Kreppel, F., and S. Kochanek. 2007. Modification of Adenovirus Gene Transfer Vectors With Synthetic Polymers: A Scientific Review and Technical Guide. Mol. Ther. 16:16-29.
13. Low, P. S., and S. A. Kularatne. 2009. Folate-targeted therapeutic and imaging agents for cancer. Curr. Opin. Chem. Biol. 13:256-262.
14. Luchansky, S. J., H. C. Hang, E. Saxon, J. R. Grunwell, C. Yu, D. H. Dube, C. R. Bertozzi, C. L. Yuan, and T. L. Reiko. 2003. Constructing Azide-Labeled Cell Surfaces Using Polysaccharide Biosynthetic Pathways, p. 249-272, Methods in Enzymol., vol. Volume 362. Academic Press.
15. Magnusson, M. K. H., S.S.; Henning, P.; Boulanger, P.; Lindholm, L. 2002. Genetic retargeting of adenovirus vectors: functionality of targeting ligands and their influence on virus viability. J. Gene Med. 4:356-370.
16. Marsh, M. P., S. K. Campos, M. L. Baker, C. Y. Chen, W. Chiu, and M. A. Barry. 2006. Cryoelectron Microscopy of Protein IX-Modified Adenoviruses Suggests a New Position for the C Terminus of Protein IX. J. Virol. 80:11881-11886.
17. Murakami, P., and M. T. McCaman. 1999. Quantitation of Adenovirus DNA and Virus Particles with the PicoGreen Fluorescent Dye. Anal. Biochem. 274:283-288.
18. Nemerow, G. R., L. Pache, V. Reddy, and P. L. Stewart. 2009. Insights into adenovirus host cell interactions from structural studies. Virology 384:380-388.
19. Oh, I. K., H. Mok, and T. G. Park. 2006. Folate Immobilized and PEGylated Adenovirus for Retargeting to Tumor Cells. Bioconj. Chem. 17:721-727.
20. Parrott, M. B., K. E. Adams, G. T. Mercier, H. Mok, S. K. Campos, and M. A. Barry. 2003. Metabolically Biotinylated Adenovirus for Cell Targeting, Ligand Screening, and Vector Purification. Mol Ther 8:688-700.
21. Russell-Jones, G., K. McTavish, J. McEwan, J. Rice, and D. Nowotnik 2004. Vitamin-mediated targeting as a potential mechanism to increase drug uptake by tumours. J. Inorg. Biochem. 98:1625-1633.
22. Rux, J. J., P. R. Kuser, and R. M. Burnett. 2003. Structural and Phylogenetic Analysis of Adenovirus Hexons by Use of High-Resolution X-Ray Crystallographic, Molecular Modeling, and Sequence-Based Methods. J. Virol. 77:9553-9566.
23. Saban, S. D., R. R. Nepomuceno, L. D. Gritton, G. R. Nemerow, and P. L. Stewart. 2005. CryoEM Structure at 9 A Resolution of an Adenovirus Vector Targeted to Hematopoietic Cells. Journal of Molecular Biology 349:526-537.
24. Saban, S. D., M. Silvestry, G. R. Nemerow, and P. L. Stewart. 2006. Visualization of {alpha}-Helices in a 6-Angstrom Resolution Cryoelectron Microscopy Structure of Adenovirus Allows Refinement of Capsid Protein Assignments. J. Virol. 80:12049-12059.
25. San Martin, C., J. N. Glasgow, A. Borovjagin, M. S. Beatty, E. A. Kashentseva, D. T. Curiel, R. Marabini, and I. P. Dmitriev. 2008. Localization of the N-Terminus of Minor Coat Protein Ma in the Adenovirus Capsid. Journal of Molecular Biology 383:923-934.
26. Varghese, R., Y. Mikyas, P. L. Stewart, and R. Ralston. 2004. Postentry Neutralization of Adenovirus Type 5 by an Antihexon Antibody. J. Virol. 78:12320-12332.
27. Waehler, R., S. J. Russell, and D. T. Curiel. 2007. Engineering targeted viral vectors for gene therapy. Nat. Rev. Genet. 8:573-587.
28. Wu, E., L. Pache, D. J. Von Seggern, T.-M. Mullen, Y. Mikyas, P. L. Stewart, and G. R. Nemerow. 2003. Flexibility of the Adenovirus Fiber Is Required for Efficient Receptor Interaction. J. Virol. 77:7225-7235.
29. Wu, H., and D. T. Curiel. 2008. Fiber-modified Adenoviruses for Targeted Gene Therapy, p. 113-132, Gene Therapy Protocols.

### REFERENCES IN EXAMPLES 8-14

(1) Waehler, R.; Russell, S. J.; Curiel, D. T. Nat. Rev. Genet. 2007, 8, 573.
(2) Hedley, S.; Chen, J.; Mountz, J.; Li, J.; Curiel, D.; Korokhov, N.; Kovesdi, I. Cancer Immunol. Immunother. 2006, 55, 1412.
(3) Matthews, Q.; Yang, P.; Wu, Q.; Belousova, N.; Rivera, A.; Stoff-Khalili, M.; Waehler, R.; Hsu, H.-C.; Li, Z.; Li, J.; Mountz, J.; Wu, H.; Curiel, D. Virol. J. 2008, 5, 98.
(4) Magnusson, M. K. H., S. S.; Henning, P.; Boulanger, P.; Lindholm, L. J. Gene Med. 2002, 4, 356.
(5) Henning, P.; Lundgren, E.; Carlsson, M.; Frykholm, K.; Johannisson, J.; Magnusson, M. K.; Tang, E.; Franqueville, L.; Hong, S. S.; Lindholm, L.; Boulanger, P. J. Gen. Virol. 2006, 87, 3151.
(6) Croyle, M. A.; Yu, Q.-C.; Wilson, J. M. Hum. Gene Ther. 2000, 11, 1713.
(7) Kreppel, F.; Kochanek, S. Mol. Ther. 2007, 16, 16.
(8) Kreppel, F.; Gackowski, J.; Schmidt, E.; Stefan, K. Mol. Ther. 2005, 12, 107.
(9) Carrico, Z. M.; Romanini, D. W.; Mehl, R. A.; Francis, M. B. Chem. Commun. 2008, 1205.
(10) Strable, E.; Prasuhn, D. E.; Udit, A. K.; Brown, S.; Link, A. J.; Ngo, J. T.; Lander, G.; Quispe, J.; Potter, C. S.; Carragher, B.; Tirrell, D. A.; Finn, M. G. Bioconj. Chem. 2008, 19, 866.
(11) Gupta, S. S.; Raja, K. S.; Kaltgrad, E.; Strable, E.; Finn, M. G. Chem. Comm. 2005, 4315.
(12) Bruckman, M. A.; Kaur, G.; Lee, L. A.; Xie, F.; Sepulveda, J.; Breitenkamp, R.; Zhang, X.; Joralemon, M.; Russell, T. P.; Emrick, T.; Wang, Q. ChemBioChem 2008, 9, 519.
(13) Hong, V.; Presolski, S.; Ma, C.; Finn, M. Angewandte Chemie International Edition 2009, 48, 9879.
(14) Chang, P. V.; Prescher, J. A.; Hangauer, M. J.; Bertozzi, C. R. J. Am. Chem. Soc. 2007, 129, 8400.
(15) Agard, N. J.; Bertozzi, C. R. Acct. Chem. Res. 2009, 42, 788.
(16) Christoph, V.; Stefan, K. J. Gene Med. 2004, 6, S164.
(17) Mullis, K. G.; Haltiwanger, R. S.; Hart, G. W.; Marchase, R. B.; Engler, J. A. J. Virol. 1990, 64, 5317.
(18) Cauet, G.; Strub, J.-M.; Leize, E.; Wagner, E.; Dorsselaer, A. V.; Lusky, M. Biochem. 2005, 44, 5453.
(19) Vocadlo, D. J.; Hang, H. C.; Kim, E.-J.; Hanover, J. A.; Bertozzi, C. R. Proc. Natl. Acad. Sci. USA 2003, 100, 9116.
(20) Agard, N. J.; Baskin, J. M.; Prescher, J. A.; Lo, A.; Bertozzi, C. R. ACS Chem. Biol. 2006, 1, 644.
(21) Gupta, S. S.; Kuzelka, J.; Singh, P; Lewis, W. G.; Manchester, M.; Finn, M. G. Bioconj. Chem. 2005, 16, 1572.
(22) Kiick, K. L.; Saxon, E.; Tirrell, D. A.; Bertozzi, C. R. Proc. Natl. Acad. Sci. USA 2002, 99, 19.
(23) Saxon, E.; Bertozzi, C. R. Science 2000, 287, 2007.
(24) Glasgow, J. N.; Everts, M.; Curiel, D. T. Cancer Gene Ther. 2006, 13, 830.
(25) Yamamoto, M.; Curiel, D. T. Mol. Ther. 2009, 18, 243.
(26) Jogler, C.; Hoffmann, D.; Theegarten, D.; Grunwald, T.; Uberla, K.; Wildner, O. J. Virol. 2006, 80, 3549.
(27) Shashkova, E. V.; May, S. M.; Barry, M. A. Virology 2009, 394, 311.
(28) Low, P. S.; Kularatne, S. A. Curr. Opin. Chem. Biol. 2009, 13, 256.
(29) Oh, I. K.; Mok, H.; Park, T. G. Bioconj. Chem. 2006, 17, 721.
(30) Russell-Jones, G.; McTavish, K.; McEwan, J.; Rice, J.; Nowotnik, D. J. Inorg. Biochem. 2004, 98, 1625.

### REFERENCES IN EXAMPLE 15

(1) Yamamoto, M.; Curiel, D. T. Mol. Ther. 2009, 18, 243.
(2) Pesonen, S.; Kangasniemi, L.; Hemminki, A. Mol. Pharm. 2010, 8, 12.
(3) Passer, B. J.; Castelo-Branco, P.; Buhrman, J. S.; Varghese, S.; Rabkin, S. D.; Martuza, R. L. Cancer Gene Ther 2009, 16, 551.
(4) Huang, B.; Sikorski, R.; Kim, D. H.; Thome, S. H. Gene Ther 2011, 18, 164.
(5) Tseng, J. C.; Granot, T.; DiGiacomo, V.; Levin, B.; Meruelo, D. Cancer Gene Ther 2010, 17, 244.
(6) Ottolino-Perry, K.; Diallo, J.-S.; Lichty, B. D.; Bell, J. C.; Andrea McCart, J. Mol Ther 2009, 18, 251.
(7) Yu, D.C.; Chen, Y.; Dilley, J.; Li, Y. H.; Embry, M.; Zhang, H.; Nguyen, N.; Amin, P.; Oh, J.; Henderson, D. R. Cancer Res. 2001, 61, 517.
(8) Zhang, J.; Ramesh, N.; Chen, Y.; Li, Y. H.; Dilley, J.; Working, P.; Yu, D. C. Cancer Res. 2002, 62, 3743.
(9) Cheong, S.C.; Wang, Y.; Meng, J. H.; Hill, R.; Sweeney, K.; Kim, D.; Lemoine, N. R.; Hallden, G. Cancer Gene Therapy 2008, 15, 40.
(10) Hassan, M.; Braam, S. R.; Kruyt, F. A. E. Cancer Gene Therapy 2006, 13, 1105.
(11) Li, Y. M.; Song, S. T.; Jiang, Z. F.; Zhang, Q.; Qu, Y. M.; Su, C. Q.; Zhao, C. H.; Li, Z. Q.; Ge, F. J.; Qian, Q. J. Chinese Journal of Cancer Research 2007, 19, 76.
(12) Radhakrishnan, S.; Miranda, E.; Ekblad, M.; Holford, A.; Pizarro, M. T.; Lemoine, N. R.; Hallden, G. Human Gene Therapy 2010, 21, 1311.
(13) Nagano, S.; Perentes, J. Y.; Jain, R. K.; Boucher, Y. Cancer Res. 2008, 68, 3795.
(14) Ojima, I.; Chen, J.; Sun, L.; Borella, C. P.; Wang, T.; Miller, M. L.; Lin, S, N.; Geng, X. D.; Kuznetsova, L. R.; Qu, C. X.; Gallager, D.; Zhao, X. R.; Zanardi, I.; Xia, S. J.; Horwitz, S. B.; Mallen-St Clair, J.; Guerriero, J. L.; Bar-Sagi, D.; Veith, J. M.; Pera, P.; Bernacki, R. J. J. Med. Chem. 2008, 51, 3203.
(15) Kuznetsova, L.; Chen, J.; Sun, L.; Wu, X. Y.; Pepe, A.; Veith, J. A.; Pera, P.; Bernacki, R. J.; Ojima, I. Bioorg. Med. Chem. Lett. 2006, 16, 974.
(16) Chen, S. Y.; Zhao, X. R.; Chen, J. Y.; Chen, J.; Kuznetsova, L.; Wong, S. S.; Ojima, I. Bioconjugate Chem. 2010, 21, 979.
(17) Botchkina, G.; Zuniga, E.; Das, M.; Wang, Y.; Wang, H.; Zhu, S.; Savitt, A.; Rowehl, R.; Leyfman, Y.; Ju, J.; Shroyer, K.; Ojima, I. Mol Cancer 2010, 9, 192.
(18) Banerjee, P. S.; Ostapchuk, P.; Hearing, P.; Carrico, I. JACS 2010, 132, 13615.
(19) Boyce, M.; Carrico, I. S.; Ganguli, A. S.; Yu, S.-H.; Hangauer, M. J.; Hubbard, S. C.; Kohler, J. J.; Bertozzi, C. R. PNAS 2011, 108, 3141.
(20) Johnson, J. A.; Lu, Y. Y.; Van Deventer, J. A.; Tirrell, D. A. Curr Op Chem. Biol. 2010, 14, 774.
(21) Tollefson, A. E.; Hermiston, T. W.; Wold, W. S. 1998; Vol. 21, p 1.
(22) Luchansky, S. J.; Hang, H. C.; Saxon, E.; Grunwell, J. R.; Danielle, C. Y.; Dube, D. H.; Bertozzi, C. R. In Recognition of Carbohydrates in Biological Systems Pt A: General Procedures; Academic Press Inc: San Diego, 2003; Vol. 362, p 249.
(23) Gupta, S. S.; Kuzelka, J.; Singh, P.; Lewis, W. G.; Manchester, M.; Finn, M. G. Bioconj. Chem. 2005, 16, 1572.
(24) Reddy, V. S.; Natchiar, S. K.; Stewart, P. L.; Nemerow, G. R. Science 2010, 329, 1071.
(25) Jogler, C.; Hoffmann, D.; Theegarten, D.; Grunwald, T.; Uberla, K.; Wildner, O. J. Virol. 2006, 80, 3549.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiment, it should be understood that the invention as claimed should not be unduly limited to such specific embodiments. Indeed, various modifications of the described modes for carrying out the invention which are obvious to those skilled in the art and in fields related thereto are intended to be within the scope of the following claims.

1. A modified infectious virus having an external surface covalently linked to at least one heterologous unnatural moiety selected from unnatural amino acid and unnatural saccharide.

2. The virus of claim 1, wherein said infectious virus is replication competent.

3. The virus of claim 1, wherein said infectious virus is capable of integrating into the genome of a cell that is susceptible to said virus, and wherein said virus is not replication competent.

4. The virus of claim 1, wherein said external surface comprises one or more of a viral structural protein and a viral structural glycoprotein.

5. The virus of claim 1, wherein at least one of said unnatural amino acid and said unnatural saccharide comprises one or more chemically reactive group selected from azido group, alkyne group, and a group as shown in FIG. 23.

6. The virus of claim 5, wherein said chemically reactive group comprises an azido group.

7. The virus of claim 6, wherein said unnatural amino acid that comprises said azido group comprises azidohomoalanine (AHA).

8. The virus of claim 6, wherein said unnatural saccharide that comprises said azido group comprises GlcNAz.

9. The virus of claim 1, wherein said unnatural moiety is covalently linked to a molecule of interest.

10. A kit comprising a virus and at least one heterologous unnatural moiety selected from unnatural amino acid and unnatural saccharide.

11. A method for producing a modified infectious virus having an external surface covalently linked to an unnatural moiety, comprising
i) contacting a virus with a host cell susceptible to said virus, wherein said contacting is under conditions for infection of said host cell by said virus to produce an infected cell that comprises said virus, and
ii) contacting said infected cell with at least one unnatural moiety selected from unnatural amino acid and unnatural saccharide, wherein said contacting is under conditions for covalently linking an external surface of said virus, that is comprised in said infected cell, with said unnatural moiety.

12. The method of claim 11, wherein said susceptible host cell is permissive to said virus, and said modified infectious virus is released from said treated cell.

13. The method of claim 11, further comprising determining the level of infection of said treated cells by said modified infectious virus.

14. The method of claim 11, further comprising detecting substantially the same level of infectivity of said modified infectious virus as a control virus that lacks said unnatural moiety.

15. The method of claim 11, further comprising determining the level of replication of said modified infectious virus in said treated cells.

16. The method of claim 11, further comprising detecting substantially the same or greater level of replication of said modified infectious virus compared to a control virus that lacks said unnatural moiety.

17. (canceled)

18. A method for reducing one or more symptoms of disease in a subject, comprising administering to said subject a therapeutic amount of the virus of claim 1 to produce a treated subject, wherein said unnatural moiety is covalently linked to a molecule of interest.

19. The method of claim 18, further comprising determining the level of one or more symptoms of said disease in said treated subject.

20. The method of claim 18, wherein said administering is under conditions for integration of said virus into the genome of one or more cells in said treated subject.

21. The method of claim 18, wherein said disease comprises cancer.

22. (canceled)
